SEQUENCE LISTING (18 sequences)

SEQ ID NO: 1 - DNA, 11 bp, Artificial Sequence
  VSATF1_Q6 gene set sequence
cnntgacgtma

SEQ ID NO: 2 - DNA, 12 bp, Artificial Sequence
  VSCREB_02 gene set sequence
nngntgacgtnn

SEQ ID NO: 3 - DNA, 12 bp, Artificial Sequence
  VSCREB_Q2 gene set sequence
nstgacgtaann

SEQ ID NO: 4 - DNA, 14 bp, Artificial Sequence
  VSCREB_Q2_1 gene set sequence
nntkacgtcannns

SEQ ID NO: 5 - DNA, 12 bp, Artificial Sequence
  VSCREB_Q4_01 gene set sequence
nstgacgtmann

SEQ ID NO: 6 - DNA, 11 bp, Artificial Sequence
  VSATF1_Q6 gene set sequence
cyytgacgtca

SEQ ID NO: 7 - DNA, 20 bp, Artificial Sequence
  NR4A2 forward primer
gttcaggcgcagtatgggtc

SEQ ID NO: 8 - DNA, 21 bp, Artificial Sequence
  NR4A2 reverse primer
agagtggtaactgtagctctg

SEQ ID NO: 9 - DNA, 19 bp, Artificial Sequence
  NR4A1 forward primer
atgccctgtatccaagccc

SEQ ID NO: 10 - DNA, 20 bp, Artificial Sequence
  NR4A1 reverse primer
gtgtagccgtccatgaaggt

SEQ ID NO: 11 - DNA, 18 bp, Artificial Sequence
  DUSP6 forward primer
ctgccgggcgttctacct

SEQ ID NO: 12 - DNA, 21 bp, Artificial Sequence
  DUSP6 reverse primer
ccagccaagcaatgtaccaag

SEQ ID NO: 13 - DNA, 19 bp, Artificial Sequence
  MITF forward primer
tgcccaggcatgaacacac

SEQ ID NO: 14 - DNA, 22 bp, Artificial Sequence
  MITF reverse primer
tgggaaaaatacacgctgtgag

SEQ ID NO: 15 - DNA, 20 bp, Artificial Sequence
  FOS forward primer
cactccaagcggagacagac

SEQ ID NO: 16 - DNA, 21 bp, Artificial Sequence
  FOS reverse primer
aggtcatcagggatcttgcag

SEQ ID NO: 17 - DNA, 22 bp, Artificial Sequence
  TBP forward primer
cccgaaacgccgaatataatcc

SEQ ID NO: 18 - DNA, 22 bp, Artificial Sequence
  TBP reverse primer
gactgttcttcactcttggctc

(19) United States
(12) Patent Application Publication
(10) Pub. No.: US 2015/0141470 A1
(43) Pub. Date: May 21, 2015

(54) DIAGNOSTIC AND TREATMENT METHODS IN PATIENTS HAVING OR AT RISK OF DEVELOPING RESISTANCE TO CANCER THERAPY

(71) Applicants: The Broad Institute, Inc., Cambridge, MA (US); Dana-Farber Cancer Institute, Inc., Boston, MA (US)

(72) Inventors: Levi A. Garraway, Newton, MA (US); Cory M. Johannessen, Roslindale, MA (US)

(73) Assignees: The Broad Institute, Inc., Cambridge, MA (US); Dana-Farber Cancer Institute, Inc., Boston, MA (US)

(21) Appl. No.: 14/399,085

(22) PCT Filed: May 8, 2013
(86) PCT No.: PCT/US13/40078

§ 371 Date: Nov. 5, 2014

Related U.S. Application Data
(60) Provisional application No. 61/644,309, filed on May 8, 2012.
(60) Provisional application No. 61/780,032, filed on Mar. 13, 2013.
(60) Provisional application No. 61/783,427, filed on Mar. 14, 2013.

(51) Int. Cl. G01N 33/50; A61K 45/06; A61K 31/435; A61K 31/404; A61K 31/16

(52) U.S. Cl. CPC G01N 33/5008; A61K 31/404; A61K 31/16; A61K 31/435; A61K 45/06
USPC 514/357; 506/10; 514/415; 514/616; 422/500

(57) ABSTRACT

A method of identifying a subject having cancer who is likely to benefit from treatment with a combination therapy with a MAPK pathway inhibitor, such as a RAF inhibitor, MEK inhibitor, or ERK inhibitor, and a GEF or HDAC inhibitor is provided. A method of treating cancer in a subject in need thereof is also provided and includes administering to the subject an effective amount of a MAPK inhibitor, such as a RAF inhibitor, MEK inhibitor, or ERK inhibitor, and an effective amount of a GEF or HDAC inhibitor. A method of identifying targets that confers resistance to a MAPK pathway inhibitor is also provided.

### CROSS-REFERENCE TO RELATED APPLICATIONS

This application claims the benefit of U.S. Provisional Application No. 61/644,309, filed May 8, 2012, U.S. Provisional Application No. 61/780,032, filed Mar. 13, 2013, and U.S. Provisional Application No. 61/783,427, filed Mar. 14, 2013. The entire contents of each of these referenced provisional applications are incorporated by reference herein.

### FEDERALLY SPONSORED RESEARCH OR DEVELOPMENT

This invention was made with government support under federal grant numbers K08 CA115927 and 1 DP20D002750 awarded by National Institutes of Health. The government has certain rights in the invention.

### BACKGROUND OF INVENTION

Oncogenic mutations in the serine/threonine kinase B-RAF (also known as BRAF) are found in 50-70% of malignant melanomas. (Davies, H. et al., Nature 417, 949-954 (2002).) Pre-clinical studies have demonstrated that the B-RAF(V600E) mutation predicts a dependency on the mitogen-activated protein kinase (MAPK) signaling cascade in melanoma (Hoeflich, K. P. et al., Cancer Res. 69, 3042-3051 (2009); McDermott, U. et al., Proc. Natl Acad. Sci. USA 104, 19936-19941 (2007); Solit, D. B. et al. BRAF mutation predicts sensitivity to MEK inhibition. Nature 439, 358-362 (2006); Wan, P. T. et al., Cell 116, 855-867 (2004); Wellbrock, C. et al., Cancer Res. 64, 2338-2342 (2004))—an observation that has been validated by the success of RAF or MEK inhibitors in clinical trials (Flaherty, K. T. et al., N. Engl. J. Med. 363, 809-819 (2010); Infante, J. R. et al., J. Clin. Oncol. 28 (suppl.), 2503 (2010); Schwartz, G. K. et al., J. Clin. Oncol. 27 (suppl.), 3513 (2009).)

However, clinical responses to targeted anticancer therapeutics are frequently confounded by de novo or acquired resistance. (Engelman, J. A. et al., Science 316, 1039-1043 (2007); Gorre, M. E. et al., Science 293, 876-880 (2001); Heinrich, M. C. et al., J. Clin. Oncol. 24, 4764-4774 (2006); Daub, H., Specht, K. & Ullrich, A. Nature Rev. Drug Discov. 3, 1001-1010 (2004).) Accordingly, there remains a need for new methods for identification of resistance mechanisms in a manner that elucidates “druggable” targets for effective long-term treatment strategies, for new methods of identifying patients that are likely to benefit from the treatment strategies, and for methods of treating patients with the effective long-term treatment strategies.

### SUMMARY OF INVENTION

The present invention relates to the development of resistance to therapeutic agents in the treatment of cancer and identification of targets that confer resistance to treatment of cancer. The present invention also relates to identification of further drug targets for facilitating an effective long-term treatment strategy and to identifying patients that would benefit from such treatment.

The invention therefore provides methods of identifying subjects at risk of developing resistance to particular anti-cancer therapies prior to the manifestation of such resistance, methods of identifying the molecular basis of observed resistance in subjects receiving particular anti-cancer therapies, thereby informing a medical practitioner of future treatment course, and methods of treating subjects at risk of developing or having resistance to particular anti-cancer therapies based on a particular molecular profile.

The invention provides diagnostic methods based on increased levels or activities of one or more markers relative to normal controls. The increased levels may be increased gene number (or copy), or increased mRNA expression, or increased protein levels. The increased levels or increased activities may be due to a mutation in the marker gene. Accordingly the invention also contemplates assaying for a mutation in the marker gene locus. Markers of interest include guanine nucleotide exchange factor factors (GEFs), G protein coupled receptors (GPCRs), transcription factors, serine/threonine kinases, ubiquitin machinery proteins, adaptor proteins, protein tyrosine kinases, receptor tyrosine kinases, protein binding proteins, cytoskeletal proteins, and RNA binding proteins. These methods can be used to identify subjects who should be treated with an HDAC or GEF inhibitor before or after another anti-cancer therapy, or who should be treated with an HDAC or GEF inhibitor along with another anti-cancer therapy. The subject may or may not have been treated with an anti-cancer therapy prior to such diagnosis. The subject may or may not have demonstrated resistance, including partial or total resistance, to an anti-cancer therapy prior to the diagnostic method being performed.

Aspects of the invention relate to a method comprising: (a) assaying, in cancer cells from a subject having cancer, a gene copy number, mRNA or protein level, or activity level of a marker selected from:
(i) GEFs selected from the group consisting of ARHGEF2, ARHGEF3, ARHGEF9, ARHGEF19, MCF2L, NGEF, VAV1, PLEKHG3, PLEKHG5, PLEKHG6, IQSEC1, TBC1D3G, SPATA13, RASGRP2, RASGRP3, and RASGRP4,
(ii) GPCRs that activate production of cyclic AMP,
(iii) GPCR pathway components selected from the group consisting of PKA, FOS, NR4A1, NR4A2, MITF, and a PKA-activated transcription factor that activates FOS, NR4A1, NR4A2, and MITF,
(iv) transcription factors selected from the group consisting of POU51, HOXD9, EBF1, HNF4A, SP6, ESRRG, TFEB, FOXA3, FOS, MITF, FOXJ1, XBP1, NR4A1, ETV1, HEY1, KLF6, HEY2, JUNB, SP8, OLIG3, PURG, FOXP2, YAP1, NFE2L1, TLE1, PASD1, TP53, WWTR1, SATB2, NR4A2, HAND2, GCM2, SHOX2, NANOG, CRX, ZNF423, ISX, ETS2, SIM2, MAFB, MYOD1, and HOXC11,
(v) serine/threonine kinases selected from the group consisting of PRKACA, RAF1, NF2, PRKCE, PAK3, and MOS,
(vi) ubiquitin machinery proteins selected from the group consisting of FBX05, TNFAIP1, KLHL10, ARIH1, and TRIM50,
(vii) adaptor proteins selected from the group consisting of CRKL, CRK, TRAF3IP1, FRS3, AND SQSTM1,
(viii) protein tyrosine kinases selected from the group consisting of HCK, BTK, LCK, SRC, and LYNp,
(ix) receptor tyrosine kinases selected from the group consisting of FGR, FGFR2, AXL, and TYRO3,
(x) protein binding proteins selected from the group consisting of CARD9 and WDR5,
(xi) cytoskeletal proteins selected from the group consisting of PVRL1 and TEKT5,
(xii) RNA binding proteins selected from the group consisting of SAMD4B and SAMD4A, and
(xiii) VPS28, IFNA10, KLHL34, TNFRSF13B, CYP2E1, BRMS1L, ADAP2, MLYCD, MAGEA9, RIT2, and KCTD1;
   (b) comparing the gene copy number, mRNA or protein level, or activity level of the marker in the cancer cells with a gene copy number, mRNA or protein level, or activity level of the marker in normal cells, and (c) identifying a subject having cancer cells with increased gene copy number, mRNA or protein level, or activity level of the marker relative to normal cells as a subject who is at risk of developing resistance to a MAPK pathway inhibitor. In some embodiments, the method further comprises (d) assaying a nucleic acid sample obtained from the cancer cells for presence of a B-RAF^{V600E} mutation.

Another aspect of the invention relates to a method comprising (a) assaying, in cancer cells from a subject having cancer, a gene copy number, mRNA or protein level, or activity level of a marker selected from:
(i) GPCRs that activate production of cyclic AMP, and
(ii) GPCR pathway components selected from the group consisting of PKA, FOS, NR4A1, NR4A2, MITF, and a PKA-activated transcription factor that activates FOS, NR4A1, NR4A2, and MITF,
   (b) comparing the gene copy number, mRNA or protein level, or activity level of the marker in the cancer cells with a gene copy number, mRNA or protein level, or activity level of the marker in normal cells, and (c) identifying a subject having cancer cells with increased gene copy number, mRNA or protein level, or activity level of the marker relative to normal cells as a subject (i) who is at risk of developing resistance to a MAPK pathway inhibitor, (ii) who is likely to benefit from treatment with an HDAC inhibitor, (iii) who is likely to benefit from treatment with a combination therapy comprising an HDAC inhibitor, and/or (iv) who is likely to benefit from treatment with a combination therapy comprising a MAPK pathway inhibitor and an HDAC inhibitor. In some embodiments, the GPCRs that activate production of cyclic AMP are selected from the group consisting of GPR4, GPR3, GPBAR1, HTR2C, MAS1, ADORA2A, GPR161, GPR52, GPR101, and GPR119. In some embodiments, the PKA-activated transcription factor that activates FOS, NR4A1, NR4A2, and MITF is selected from the group consisting of CREB1, ATF4, ATF1, CREB3, CREB5, CREB3L1, CREB3L2, CREB3L3, and CREB3L4. In some embodiments, the method further comprises (d) assaying a nucleic acid sample obtained from the cancer cells for presence of a B-RAF^{V600E} mutation.

In some embodiments, the cancer is selected from the group consisting of melanoma, breast cancer, colorectal cancer, glioma, lung cancer, ovarian cancer, sarcoma and thyroid cancer. In some embodiments, the cancer is melanoma. In some embodiments, the cancer cells comprise a mutation in B-RAF. In some embodiments, the cancer cells comprise a B-RAF^{V600E} mutation.

In some embodiments, the subject has received a therapy comprising a MAPK pathway inhibitor. In some embodiments, the subject has manifest resistance to the MAPK pathway inhibitor.

In some embodiments, the MAPK pathway inhibitor is a RAF inhibitor. In some embodiments, the MAPK pathway inhibitor is a pan-RAF inhibitor. In some embodiments, the MAPK pathway inhibitor is a selective RAF inhibitor. In some embodiments, RAF inhibitor is selected from the group consisting of RAF265, sorafenib, dabrafenib (GSK2118436), SB590885, PLX 4720, PLX4032, GDC-0879 and ZM 336372.

In some embodiments, the MAPK pathway inhibitor is a MEK inhibitor. In some embodiments, the MEK inhibitor is selected from the group consisting of CI-1040/PD184352, AZD6244, PD318088, PD98059, PD334581, RDEA119, 6-Methoxy-7-(3-morpholin-4-yl-propoxy)-4-(4-phenoxy-phenylamino)-quinoline-3-carbonitrile and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, trametinib (GSK1120212), and ARRY-438162.

In some embodiments, the MAPK pathway inhibitor is two MAPK pathway inhibitors, and wherein one of a first of the two MAPK inhibitors is a RAF inhibitor and a second of the two MAPK inhibitors is a MEK inhibitor.

In some embodiments, the MAPK pathway inhibitor is an ERK inhibitor. In some embodiments, the ERK inhibitor is selected from the group consisting of VTX11e, AEZS-131, PD98059, FR180204, and FR148083.

In some embodiments, the HDAC inhibitor is selected from the group consisting of Vorinostat, CI-994, Entinostat, BML-210, M344, NVP-LAQ824, Panobinostat, Mocetinostat, and Belinostat.

In some embodiments, the normal cells are from the subject having cancer. In some embodiments, the normal cells are from a subject that does not have cancer.

Other aspects of the invention relate to a method, comprising administering an effective amount of an HDAC inhibitor alone or together with (a) an effective amount of a RAF inhibitor, (b) an effective amount of a MEK inhibitor, (c) an effective amount of an ERK inhibitor, and/or (d) an effective amount of a RAF inhibitor and a MEK inhibitor to a subject with cancer having an increased gene copy number, mRNA or protein level, or activity of a marker selected from: (i) GPCRs that activate production of cyclic AMP, and (ii) GPCR pathway components selected from the group consisting of PKA, FOS, NR4A1, NR4A2, MITF, and a PKA-activated transcription factor that activates FOS, NR4A1, NR4A2, and MITF.

In yet other aspects, the invention relates to a method, comprising administering to a subject having cancer an effective amount of an HDAC inhibitor together with (a) an effective amount of a RAF inhibitor, (b) an effective amount of a MEK inhibitor, (c) an effective amount of an ERK inhibitor, and/or (d) an effective amount of a RAF inhibitor and a MEK inhibitor. In some embodiments, the subject has cancer cells comprising a mutation in B-RAF. In some embodiments, the subject has cancer cells comprising a B-RAF^{V600E} mutation. In some embodiments, the RAF inhibitor is selected from the group consisting of RAF265, sorafenib, dabrafenib (GSK2118436), SB590885, PLX 4720, PLX4032, GDC-0879 and ZM 336372. In some embodiments, the MEK inhibitor is selected from the group consisting of CI-1040/PD184352, AZD6244, PD318088, PD98059, PD334581, RDEA119, 6-Methoxy-7-(3-morpholin-4-yl-propoxy)-4-(4-phenoxy-phenylamino)-quinoline-3-carbonitrile and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, trametinib (GSK1120212), and ARRY-438162. In some embodiments, the ERK inhibitor is selected from the group consisting of VTX11e, AEZS-131, PD98059, FR180204, and FR148083. In some embodiments, the HDAC inhibitor is selected from the group consisting of Vorinostat, CI-994, Entinostat, BML-210, M344, NVP-LAQ824, Panobinostat, Mocetinostat, and Belinostat.

In some embodiments, the subject has innate resistance to the RAF inhibitor or is likely to develop resistance to the RAF inhibitor. In some embodiments, the subject has innate resistance to the MEK inhibitor or is likely to develop resistance to the MEK inhibitor. In some embodiments, the cancer is selected from the group consisting of melanoma, breast cancer, colorectal cancer, glioma, lung cancer, ovarian cancer, sarcoma and thyroid cancer. In some embodiments, the cancer is melanoma.

Another aspect of the invention relates to a method of identifying a marker that confers resistance to a MAPK pathway inhibitor, the method comprising: culturing cells having sensitivity to a MAPK pathway inhibitor; expressing a plurality of ORF clones in the cell cultures, each cell culture expressing a different ORF clone; exposing each cell culture to the MAPK pathway inhibitor; and identifying cell cultures having greater viability than a control cell culture after exposure to the MAPK pathway inhibitor to identify one or more ORF clones that confers resistance to the MAPK pathway inhibitor. In some embodiments, the cultured cells have sensitivity to a RAF inhibitor. In some embodiments, the cultured cells have sensitivity to a MEK inhibitor. In some embodiments, the cultured cells have sensitivity to an ERK inhibitor. In some embodiments, the cultured cells comprise a B-RAF mutation. In some embodiments, the cultured cells comprise a B-RAF^{V600E} mutation. In some embodiments, the cultured cells comprise a melanoma cell line.

Other aspects of the invention relate to a device comprising a sample inlet and a substrate, wherein the substrate comprises a binding partner for a marker selected from:
(i) GEFs selected from the group consisting of ARHGEF2, ARHGEF3, ARHGEF9, ARHGEF19, MCF2L, NGEF, VAV1, PLEKHG3, PLEKHG5, PLEKHG6, IQSEC1, TBC1D3G, SPATA13, RASGRP2, RASGRP3, and RASGRP4,
(ii) GPCRs that activate production of cyclic AMP,
(iii) GPCR pathway components selected from the group consisting of PKA, FOS, NR4A1, NR4A2, MITF, and a PKA-activated transcription factor that activates FOS, NR4A1, NR4A2, and MITF,
(iv) transcription factors selected from the group consisting of POU51, HOXD9, EBF1, HNF4A, SP6, ESRRG, TFEB, FOXA3, FOS, MITF, FOXJ1, XBP1, NR4A1, ETV1, HEY1, KLF6, HEY2, JUNB, SP8, OLIG3, PURG, FOXP2, YAP1, NFE2L1, TLE1, PASD1, TP53, WWTR1, SATB2, NR4A2, HAND2, GCM2, SHOX2, NANOG, CRX, ZNF423, ISX, ETS2, SIM2, MAFB, MYOD1, and HOXC11,
(v) serine/threonine kinases selected from the group consisting of PRKACA, RAF1, NF2, PRKCE, PAK3, and MOS,
(vi) ubiquitin machinery proteins selected from the group consisting of FBX05, TNFAIP1, KLHL10, ARIH1, and TRIM50,
(vii) adaptor proteins selected from the group consisting of CRKL, CRK, TRAF3IP1, FRS3, AND SQSTM1,
(viii) protein tyrosine kinases selected from the group consisting of HCK, BTK, LCK, SRC, and LYNp,
(ix) receptor tyrosine kinases selected from the group consisting of FGR, FGFR2, AXL, and TYRO3,
(x) protein binding proteins selected from the group consisting of CARD9 and WDR5,
(xi) cytoskeletal proteins selected from the group consisting of PVRL1 and TEKT5,
(xii) RNA binding proteins selected from the group consisting of SAMD4B and SAMD4A, and
(xiii) VPS28, IFNA10, KLHL34, TNFRSF13B, CYP2E1, BRMS1L, ADAP2, MLYCD, MAGEA9, RIT2, and KCTD1.

In another aspect, the invention provides a method of identifying a subject having cancer who is at risk of developing resistance to a MAPK pathway inhibitor. The method includes assaying the level or activity of a guanine nucleotide exchange factor (GEF) in the subject. The level of GEF may be GEF gene level, GEF mRNA level, or GEF protein level. GEF level or activity may be assayed in cancer cells of the subject. The level or activity is then compared to a GEF level or activity in normal cells. Such normal cells may be non-cancerous cells of the subject having cancer or cells of a subject that does not have cancer. A GEF level or activity in cancerous cells that is higher than a GEF level or activity in normal cells is indicative of a subject at risk of developing resistance to a MAPK pathway inhibitor.

In another aspect, the invention provides a method of identifying a subject having cancer who is likely to benefit from treatment with GEF inhibitor alone or in combination with one or more additional therapies. The one or more additional therapies may be but are not limited to one or more MAPK pathway inhibitors such as but not limited to a RAF inhibitor and/or a MEK inhibitor. The method includes assaying a GEF gene copy number, a GEF mRNA or a GEF protein level, or a GEF activity level in cancer cells obtained from the subject, and comparing such GEF level or activity with a GEF gene copy number, a GEF mRNA or a GEF protein level, or a GEF activity level in cells obtained from a subject without the cancer or in non-cancerous cells obtained from the subject having cancer. The method then identifies subjects likely to benefit from treatment with the GEF inhibitor alone or in combination therapy as subjects having an increased GEF gene copy number, an increased GEF mRNA expression level, an increased GEF protein expression, or an increased GEF activity level compared to levels in subjects without cancer or non-cancerous cells in subjects with cancer.

In another aspect, the invention provides a method of treating cancer in a subject. The method includes administering to the subject an effective amount of one or more MAPK pathway inhibitors and an effective amount of one or more GEF inhibitors.

In another aspect, the invention provides a method of treating cancer in a subject. The method includes administering to the subject an effective amount of a RAF inhibitor, or a MEK inhibitor, or a RAF inhibitor and a MEK inhibitor, and an effective amount of a GEF inhibitor.

In another aspect, the invention provides a method of treating cancer in a subject comprising administering, to a subject having an increased GEF gene copy number, mRNA or protein level, or activity relative to a normal control, the effective amount of a GEF inhibitor and (i) an effective amount of a RAF inhibitor, (ii) an effective amount of a MEK inhibitor, or (iii) an effective amount of a RAF inhibitor and an effective amount of a MEK inhibitor. The normal control may be non-cancerous cells from the subject having cancer or it may be cells from a subject not having cancer.

In some embodiments, the GEF may be ARHGEF2, ARHGEF3, ARHGEF9, ARHGEF19, IQSEC1, MCF2L, NGEF, PLEKHG3, PLEKHG5, PLEKHG6, TBC1 D3G, SPATA13, or VAV1. The GEF inhibitor may be an aptamer, an siRNA, an shRNA, a small peptide, an antibody or antibody fragment, or a small chemical compound. Specific examples are provided herein.

The MAPK pathway inhibitor may be a RAF inhibitor such as a selective RAF inhibitor such as PLX4720, PLX4032, GDC-0879 or 885-A, or a pan-RAF inhibitor such as FAR265, sorafinib or SG590885, or it may be a MEK inhibitor such as but not limited to CI-1040/PD184352 or AZD6244.

In some embodiments, the cancer is selected from the group consisting of melanoma, breast cancer, colorectal cancers, glioma, lung cancer, ovarian cancer, sarcoma and thyroid cancer. In some embodiments, the cancer is melanoma, including metastatic and non-metastatic melanoma.

In some embodiments, the cancer cells comprise a mutation in B-RAF. In some embodiments, the cancer cells comprise a V600E B-RAF mutation.

In some embodiments, the subject has received a therapy comprising a MAPK pathway inhibitor. In some embodiments, the subject has manifest (or demonstrated) resistance to a MAPK pathway inhibitor. In some embodiments, the subject is likely to develop resistance to a MAPK pathway inhibitor. In some embodiments, the subject has innate resistance to the RAF inhibitor or is likely to develop resistance to the RAF inhibitor. In some embodiments, the subject has innate resistance to the MEK inhibitor or is likely to develop resistance to the MEK inhibitor.

In some embodiments, the MAPK pathway inhibitor is a RAF inhibitor. In some embodiments, the MAPK pathway inhibitor is a pan-RAF inhibitor. In some embodiments, the MAPK pathway inhibitor is a selective RAF inhibitor. In some embodiments, the RAF inhibitor is selected from the group consisting of RAF265, sorafenib, SB590885, PLX 4720, PLX4032, GDC-0879 and ZM 336372. In some embodiments, the MAPK pathway inhibitor is a MEK inhibitor.

In some embodiments, the GEF inhibitor is an inhibitor of ARHGEF2, ARHGEF3, ARHGEF9, ARHGEF19, MCF2L, NGEF, VAV1, PLEKHG3, PLEKHG5, PLEKHG6, IQSEC1, TBC1 D3G and/or SPATA13.

In some embodiments, the method comprises assaying the gene copy number, the mRNA or the protein level of one or more GEFs. In some embodiments, the method comprises assaying active status of one or more GTPases.

In another aspect, the invention provides a method of identifying a target that confers resistance to a first inhibitor that is a MAPK pathway inhibitor. The method includes culturing cells having sensitivity to the first inhibitor and expressing a plurality of GEF ORF clones in the cell cultures, each cell culture expressing a different GEF ORF clone. The method further includes exposing each cell culture to the first inhibitor and identifying cell cultures having greater viability than a control cell culture after exposure to the first inhibitor to identify the GEF ORF clone that confers resistance to the first inhibitor.

In some embodiments, the cultured cells have sensitivity to a RAF inhibitor. In some embodiments, the cultured cells have sensitivity to a MEK inhibitor. In some embodiments, the cultured cells comprise a B-RAF mutation. In some embodiments, the cultured cells comprise a B-RAF^{V600E} mutation. In some embodiments, the cultured cells comprise a melanoma cell line.

### BRIEF DESCRIPTION OF DRAWINGS

FIG. 1 illustrates resistance to MAPK pathway inhibition via several GEFs. ORFS indicated on the x-axis were expressed in A375. Changes in cell numbers were assays following 18 hours of treatment with PLX4720 (first bar of each quartet), AZD6244 (second bar of each quartet), PLX4720+AZD6244 (third bar of each quartet), or VTX-11E (fourth bar of each quartet). Negative controls were cells transfected with non-human genes. As compared to the negative controls, all the GEF ORFS conferred resistance, to varying degrees, on the A375 cells.

FIG. 2 illustrates the individual effect of a GEF ORF (i.e., a VAV1 ORF) and non-human ORFS (i.e., eGFP ORF, BFP ORF, and HcRed ORF) on proliferation of the A375 cell line in the presence of PLX4720, AZD6244, PLX4720 and AZD6244, or VTX-11E. The control is proliferation in the presence of DMSO alone (i.e., the carrier for the MAPK pathway inhibitors). The area under the curve (AUC) for each ORF and inhibitor pair is plotted in FIG. 1.

FIG. 3 illustrates the effect of various GEF ORF on the levels of various MAPK pathway proteins in the presence or absence of PLX4720. The negative controls are non-human eGFP and LacZ ORFS. The positive controls are MEK1^{DD} and KRAS^{G12V} ORFS, both previously shown to confer resistance to PLX4720. The A375 cells were transfected with the indicated ORFS and then cultured in the presence of 1 μM PLX4720 or DMSO alone (i.e., carrier) for 18 hours. Lysates were analyzed by immunoblot. Several of the tested GEF ORFS reconstituted ERK phosphorylation in the presence of inhibitor to levels below that achieved by MEK1^{DD} and KRAS^{G12V}. Several of the tested GEF ORFS also reconstituted MEK phosphorylation in the presence of inhibitor to levels above that achieved by MEK1^{DD} and below that achieved by KRAS^{G12V}.

FIG. 4 illustrates the effect of various GEF ORF on the levels of kinases pERK and ERK, and GTPases Rac1 and Cdc42 in the presence or absence of PLX4720. The negative ORF controls are non-human eGFP and LacZ ORFS. The positive ORF controls are MEK1^{DD} and KRAS^{G12V} ORFS, both previously shown to confer resistance to PLX4720. The A375 cells were transfected with the indicated ORFS and then cultured in the presence of (a) 1 μM PLX4720 or (b) DMSO alone (i.e., carrier) for 18 hours. Lysates were analyzed by immunoblot. As illustrated in FIG. 3, several of the tested GEF ORFS reconstituted ERK phosphorylation in the presence of inhibitor, albeit to levels below that achieved by KRAS^{G12V}. The transfected GEF ORFS did not have an effect on the levels of GTPases Rac1 and Cdc42. The level of vinculin (VINC), the control, remains steady in the presence or absence of inhibitor and transfected ORF.

FIG. 5 illustrates the effect of various GEF ORF on the levels of active GTPases, Rac1-GTP and Cdc42-GTP, in the presence or absence of PLX4720. The negative ORF controls are non-human eGFP and LacZ ORFS. The positive ORF control is KRAS^{G12V} ORFS, previously shown to confer resistance to PLX4720. The A375 cells were transfected with the indicated ORFS and then cultured in the presence of (a) 1 μM PLX4720 or (b) DMSO alone (i.e., carrier) for 18 hours. Lysates were analyzed by immunoblot. VAV1 expression resulted in higher levels of active Rac1 (i.e., Rac1-GTP) and NGEF expression resulted in higher levels of active Cdc42 (i.e., Cdc42-GTP), suggesting the specificity between these GEFs and GTPases, and the potential mechanism through which these ORFS impact resistance to the inhibitor.

FIG. 6 illustrates the effect of various GEF ORF on the levels of pERK and ERK, and cyclin D1 (CyD1) in the presence or absence of PLX4720. The negative ORF control is LacZ ORF. The positive ORF control is MEK1^{DD}, previously shown to confer resistance to PLX4720. The A375 cells were transfected with the indicated ORFS and then cultured in the presence of (a) DMSO alone (i.e., carrier), (b) 1 μM PLX4720, (c) 200 nM AZD6244, or (d) 2 μM VTX-11E for 18 hours. Lysates were analyzed by immunoblot. Expression of some GEFs increased the level of cyclin D1 in the presence of PLX4720. PAK3, a downstream target of GTPases did not appear to change the outcome in the presence of any of the inhibitors tested. The level of vinculin (VINC), the control, remains steady in the presence or absence of the inhibitors and transfected ORF.

FIG. 7A shows that a near genome-scale functional rescue screen identifies genetic modifiers of resistance to RAF, MEK and ERK inhibitors. The right panel shows A375 cells transduced with the Center for Cancer Systems Biology (CCSB)—Broad Institute Lentiviral Expression Library were treated with PLX4720 (2 μM), AZD6244 (0.2 μM), PLX4720+AZD6244 (2 μM and 0.2 μM, respectively) or VRT11E (2 μM) and assayed for viability in the presence of compound alone (x-axis) and viability in compound relative to DMSO (y-axis). Values are presented as a z-score, where a larger z-score indicates a greater degree of resistance. Genes (n=169) with normalized rescue scores greater than or equal to 2.5 (dashed line) were nominated as candidate resistance genes. Positive controls (red circles), negative controls (yellow circles) and experimental genes (black circles) are noted. The left panel shows a summary of candidate-gene protein classes shown in FIGS. 7B-7D for protein classes containing ≧2 genes. Top y-axis indicates the number of genes per class, bottom y-axis indicates the percent of genes among all candidates within a given class.

FIGS. 7B-7D show a summary of indicated controls (negative, neutral, positive) and candidate resistance genes identified in FIG. 7A, left panel, across all tested inhibitors, annotated and grouped by protein class. Coloring is based on the z-score of resistance (plate-normalized percent rescue) used to nominate candidates in FIG. 7A, left panel. ORF class is indicated along bottom of heat map (positive control, red; negative control, yellow; experimental ORF, black). Asterisk (*) identifies genes (n=2) with an empirical sequence that is significantly divergent from its annotated reference sequence. The genes with an asterisk are ADHC1 and IGHA2. For FIG. 7B, the controls and candidates listed above the heat map are, from left to right, BFP, Egfp, LacZ, Luciferase, HcRed, Neutral, MEKDD, MAP3K8, KRASV12, NR4A1, FOS, TFEB, XBP1, POU5F1, MAFB, YAP1, WWTR1, MITF, SATB2GCM2, ESRRG, ETV1, NR4A2, HNF4A, SP6, MYOD1, MEIS2, TFAP2, HAND2, FOXP3, HEY1, ASCL2, NFE2L1, MEOX2, FOXP2, HOXD9, HEY2, FOXA3, ISX, TLE1, OLIG3, ASCL4, TP53, ETS2, ZNF423, TGIF1, FOXJ1, SOX14, MYF6, PASD1, PURG, HOXC11, ZNF503, EBF1, SIM2, JUNB, CRX, KLF6, SP8, SATB1, USF1, SHOX2, and NANOG. For FIG. 7C, the candidates listed above the heat map are, from left to right, GPR101, LPAR4, GPR35, MAS1, LPAR1, GPR4, GPR132, ADCY9, GPR52, HTR2C, GPR161, ADORA2A, GPR119, GPBAR1, GNA15, GPR3, P2RY8, VAV1, NGEF, MCF2L, PLEKHG5, TBC1 D3G, ARHGEF9, ARHGEF2, PLEKHG3, RASGRP3, PLEKHG6, SPATA13, RASGRP4, IQSEC1, ARHGEF19, RAPGEF4, ARHGEF3, and RASGRP2. For FIG. 7D, the candidates listed above the heat map are, from left to right, RAF1, PRKACA, PAK3, NF2, PAK1, PRKCE, MOS, MAP3K14, FBXO5, KLHL3, TNFAIP1, TRIM62, KLHL10, KLHL2, ARIH1, TRIM50, FRS3, CRKL, SQSTM1, CRK, GAB1, TRAF3IP2, RAPSN, TEX11, CARD9, CIOA, WDR5, SRC, LCK, BTK, HCK, LYN, AHDC1, KLHL34, BEND5, WDR18, PVRL1, PCDHGB1, UNC45B, TEKT5, FGR, TYRO3, AXL, FGFR2, FGF6, CHGA, PI16, IFNA10, RIT1, RHOBTB2, RIT2, SAMD4A, SAMD4B, FXR2, PSMC5, ATAD1, ICAM3, F3, ADAP2, RGS11, KCTD17, KCTD1, SLC35A4, SLC4A2, VPS28, MAGEA9, MPPED1, PPP1CA, MECP2, EIF4H, BRMS1L, TPI1, FBP1, NASP, MLYCD, TNFRSF13B, DNAJC5B, CYP2E1, BCL2L1, CCDC150, and IGHA2.

FIG. 8 shows that comprehensive phenotypic characterization of candidate resistance genes identifies broadly validating protein classes. (A) A375 were infected with control (positive, red; negative, blue; neutral, green) and candidate (black) genes and assayed for viability relative to DMSO in the presence of 10-fold escalating doses (0.1 nM to 10 μM) of PLX4720, AZD6244, VRT11e or 2 μM PLX4720 in combination with 0.1 nM to 10 μM AZD6244 (PLX4720+AZD6244). Area under the curve (AUC) was calculated for resulting sensitivity curves and is presented as a z-score (y-axis), relative to all negative and null controls. All genes are plotted on the x-axis (Rank) in order of decreasing resistance phenotype within each (indicated) drug treatment. (B) Venn diagram showing the overlap of genes validated in A375 (as shown in a, z-score of the AUC≧1.96). The total numbers of candidates identified in the primary screens are shown in parenthesis beneath the drug conditions, whereas only validating genes are included in the Venn diagram. (C) Schematic showing the number of genes that confer resistance to single agent RAF inhibition (PLX4720), single agent MEK inhibition (AZD6244), combination RAF/MEK inhibition (PLX4720/AZD6244), and the number of RAF, MEK, RAF/MEK-inhibitor resistant genes that remain sensitive or resistant to ERK inhibition (VRT11e). (D) The ability of each gene to induce sustained ERK phosphorylation in the presence of PLX4720 (2 μM), AZD6244 (0.2 μM), PLX4720+AZD6244 (2 μM and 0.2 μM, respectively) relative to DMSO was assessed using a microwell-based immuno-assay. Only genes that showed rescue of pERK signal to ≧22% of DMSO for a given MAPK-inhibitor are shown. ERK phosphorylation for all other candidate genes is presented in FIG. 9). (E) A panel of 7 BRAFV600E-malignant melanoma cell lines were infected as in (A) and assayed for viability relative to DMSO (percent rescue) following treatment with PLX4720 (2 μM), AZD6244 (0.2 μM), PLX4720+AZD6244 (2 μM and 0.2 μM, respectively) or VRT11e (2 μM). Resulting values are represented as a z-score, relative to all negative and neutral controls. Candidates with a z-score ≧4 were considered to be validated. Only genes validating in ≧2 conditions (drug or cell line) are shown. The controls and candidates listed above the heat map are from left to right: eGFP, HcRed, Luciferase, Neutral, MEKDD, MAP3K8, KRASV12, POU5F1, HOXD9, EBF1, HNF4A, SP6, ESRRG, TFEB, FOXA3, FOS, MITF, FOXJ1, XBP1, NR4A1, ETV1, HEY1, KLF6 HEY2, JUNB, SP8, OLIG3, PURG, FOXP2, YAP1, NFE2L1, TLE1, PASD1, TP53, WWTR1, SATB2, NR4A2, HAND2, GCM2, SHOX2, NANOG, CRX, ZNF423, ISX, ETS2, SIM2, MAFB, MYOD1, HOXC11, GPR4, GPR3, GPBAR1, HTR2C, MAS1, ADORA2A, GPR161, GPR52, GPR101, GPR119, LPAR4, GPR132, LPAR1, GPR35, P2RY8, VAV1, ARHGEF3, RASGRP2, RASGRP3, ARHGEF9, RASGRP4, SPATA13, PLEKHG6, MCF2L, PLEKHG5, NGEF, PRKACA, RAF1, NF2, PRKCE, PAK3, MOS, FBX05, TNFAIP1, KLHL10, ARIH1, TRIM50, CRKL, CRK, TRAF3IP2, FRS3, SQSTM1, HCK, BTK, LCK, SRC, LYN, FGR, FGFR2, AXL, TYRO3, CARD9, WDR5, PVRL1, TEKT5, SAMD4B, SAMD4A, VPS28, IFNA10, KLH34, TNFRSF13B, CYP2E1, BRMS1L, ADAP2, MLYCD, MAGEA9, RIT2, and KCTD1. (F) Strength of resistance phenotype and depth of validation for each gene was quantified by summing the z-score of each gene across all 7 cell lines (composite rescue score), presented in (E).

FIG. 9 shows a matrix of genes ectopically expressed in A375 (horizontal axis) versus treatment condition (vertical axis) with MAPK inhibitor. Black boxes indicate gene-mediated resistance to the indicated inhibitor, white boxes indicate sensitivity. Sensitivity is defined as yielding an area under the curve z-score of <1.96, resistance is defined as z>1.96 (p<0.005). Summary of results used to generate flow-chart are found in FIG. 8C.

FIG. 10 shows drug sensitivity curves for PLX4720 (RAF inhibitor), AZD6244 (MEK inhibitor) and VRT11E (ERK inhibitor) in the panel of 8 BRAFV600E-mutant malignant melanoma cell lines used for the primary and validation screening experiments described in FIG. 8.

FIG. 11 shows identification of a comprehensive signaling network that converges on PKA/CREB to mediate resistance to RAF, MEK and ERK inhibitors. (A) Schematic outlining a hypothetical gene network nominated by functional rescue screens, whereby expression of G protein coupled receptors (GPCR) or G-proteins (GP) induce adenyl cyclase (ADCY)-mediated production of cyclic AMP (cAMP). Generation of cyclic AMP or expression of the catalytic subunit of protein kinase A (PKA) induces CREB phosphorylation at Ser133, leading to activation of downstream effectors that overlap with MAPK pathway effectors. (B) Western blot analysis of phosphorylated CREB/ATF1 (Ser133/Ser63, pCREB/pATF1, respectively), total CREB and vinculin (VINC) in WM266.4 virally transduced with the indicated expression constructs, pre-treated for 30 minutes with 30 μM IBMX before lysis. (C) Fold change in the GI50 of PLX4720, AZD6244 or VRT11e in the indicated cell lines in the presence of vehicle (DMSO, first bar of each triplet), 10 μM forskolin and 100 μM IBMX (FSK/I, second bar of each triplet) or 100 μM dbcAMP and 100 μM IBMX (cAMP/I, third bar of each triplet). Area under the curve (AUC) was used to measure sensitivity in PLX4720+AZD6244-treated cell lines and is presented as a fold-change. All values are normalized to GI50 or AUC of MAPK-pathway inhibitor in the presence of DMSO. Results are representative of 2-3 independent experiments. (D) Western blot analysis of phosphorylated CREB (Seri 33, pCREB), ATF1 (Ser63, pATF1) and ERK (Thr202/Tyr204, pERK) and total CREB, ERK, Cyclin D1 (CyD1) and vinculin (VINC) in WM266.4 following 1 hr. treatment with 10 μM forskolin and 100 μM IBMX (FSK/I) or 100 μM dbcAMP and 100 μM IBMX (cAMP/I) in the presence of vehicle (DMSO, 96 hrs) or PLX4720 (2 μM), AZD6244 (0.2 μM), PLX4720+AZD6244 (2 μM and 0.2 μM, respectively) or VRT11E (2 μM) for 96 hrs. (E) Viability of WM266.4 expressing either LacZ (control, first bar of each triplet), CREB^{R301L} (second bar of each triplet) or A-CREB (third bar of each triplet) following treatment with 10 μM forskolin and 100 μM IBMX (FSK/I) in the presence of vehicle (DMSO) or PLX4720 (2 μM), AZD6244 (0.2 μM), PLX4720+AZD6244 (2 μM and 0.2 μM, respectively) or VRT11E (2 μM). Viability is expressed as a percentage of DMSO. Error bars represent SD, n=6. (F) Western blot analysis of phosphorylated CREB (Ser133, pCREB), ATF1 (Ser63, pATF1) and ERK (Thr202/Tyr204, pERK) and total vinculin (VINC) in lysates extracted from BRAFV600E-mutant human tumors biopsied pre-initiation of treatment (P), following 10-14 days of MAPK-inhibitor treatment (on-treatment, O) or following relapse (R).

FIG. 12 shows changes in cAMP and phospho-CREB. (A) Control or candidate gene-induced cAMP production was measured following transfection of 293T with indicated expression constructs or treatment with 10 μM forskolin and 100 μM IBMX (FSK/I). cAMP levels were determined using an immuno-competition assay in the presence (right bar for each pair) or absence (left bar for each pair) of the phosphodiesterase inhibitor 3-isobutyl-1-methylxanthine (IBMX, 30 μM, 30 minutes). Error bars represent standard deviation, n=2. The lowest dashed line represents levels of cAMP in negative controls (eGFP, Luciferase, LacZ) (B) Western blot analysis of CREB phosphorylation, total CREB and vinculin (VINC) in lysates from 293T used for cAMP assay in (A), treated with 30 μM IBMX for 30 minutes.

FIG. 13 shows identification of candidate resistance genes that are transcriptional effectors of the MAPK and cAMP-pathways. (A) Candidate and neutral control genes containing cAMP response elements (CREs) were identified using gene sets extracted from MSigDB. Fold enrichment of the percent of CRE-containing genes in candidates over all genes screened for each gene set are noted. Matrix of CRE and candidate genes indicates the presence (black box) or absence (white box) of indicated CRE. Composite resistance score for each gene (summarized in FIG. 8f) is noted. The dashed line indicates a composite resistance score of 50. The sequences listed in the “Sequence” column are, from top to bottom, TGACGTMA, TGACGTYA, CNNTGACGTMA (SEQ ID NO: 1), NNGNTGACGTNN (SEQ ID NO: 2), NSTGACGTAANN (SEQ ID NO: 3), NNTKACGTCANNNS (SEQ ID NO: 4), NSTGACGTMANN (SEQ ID NO: 5), CGTCAN, CYYTGACGTCA (SEQ ID NO: 6), and TTACGTAA. (B) Quantification of TBP-normalized DUSP6, MITF, FOS, NR4A1 and NR4A2 mRNA levels using real-time quantitative PCR (relative to DMSO-treatment) following a time course of AZD6244 treatment (200 nM) for the indicated times. For each of, MITF, FOS, NR4A1, NR4A2 and DUSP6, the bars are from left to right are DMSO, 1 hour, 6 hours, 24 hours, 48 hours, and 96 hours of AZD6244 (200 nM) treatment. Error bars represent SD, n=3. (C) Western blot analysis of phosphorylated ERK (Thr202/Tyr204, pERK), MITF, ERK and vinculin (VINC) in lysates from WM266.4 cells treated in parallel with those described in (B). Arrowhead indicates the slower migrating, phosphorylated form of MITF. (D) Quantification of TBP-normalized MITF, FOS, NR4A1 and NR4A2 mRNA levels using real-time quantitative PCR (relative to DMSO-treatment) following a time course of 10 μM forskolin and 100 μM IBMX (FSK/I) treatment for the indicated times in the presence of vehicle (DMSO, 96 hrs) or AZD6244 (200 nM, 96 hrs). Error bars represent SD, n=3. (E) Western blot analysis of phosphorylated ERK (Thr202/Tyr204, pERK), CREB (Ser133, pCREB), ATF1 (Ser63, pATF1), total ERK, MITF, FOS, NR4A1, NR4A2, cyclin D1 (CyD1), actin and the MITF target genes SILVER (SLV), tyrosinase related protein 1 (TRP1) and BCL-2 in WM266.4 cells following a time course of 10 μM forskolin and 100 μM IBMX (FSK/I) treatment for the indicated times in the presence of vehicle (DMSO, 96 hrs) or AZD6244 (200 nM, 96 hrs). Genes whose ectopic expression confers resistance to MAPK-pathway inhibition in primary and validation screens are underlined.

FIGS. 14A and B shows that MITF mediates cAMP-dependent resistance to MAPK-pathway inhibition FIG. 14A(a) Cell viability of WM266.4 expressing a control shRNA (shLuciferase) or shRNAs targeting MITF treated with a RAF inhibitor (PLX4720, 2 μM), a MEK inhibitor (AZD6244, 200 nM), combinatorial RAF/MEK inhibition (PLX4720, 2 μM, AZD6244, 200 nM) or an ERK inhibitor (VRT11E, 2 μM) and concomitant treatment with either DMSO or 10 μM forskolin and 100 μM IBMX (FSK/I). The bars for each treatment from left to right are shLuc, shMITF-492, shMITF-573, shMITF-956, and shMITF-3150. Error bars represent SD, n=6. FIG. 14A(b) Western blot analysis of WM266.4 expressing the shRNA-constructs used in a or treated with 200 nM AZD6244 alone (AZD6244) or co-treated with AZD6244 and 10 μM forskolin and 100 μM IBMX (AZD6244+FSK/I FIG. 14A(c) Western blot analysis of MITF, phosphorylated ERK (Thr202/Tyr204, pERK), ERK and vinculin (VINC) in a panel of BRAFV600E-mutant malignant melanoma cell lines following treatment with AZD6244 (200 nM) for 96 hrs. in the presence of vehicle (DMSO), 10 μM forskolin and 100 μM IBMX (FSK/I) or 100 μM dbcAMP and 100 μM IBMX (cAMP/I). FIG. 14A(d) Western blot analysis of phosphorylated ERK (Thr202/Tyr204, pERK), ERK, MITF and vinculin (VINC) in WM266.4 cells following a 6 hour treatment with 10 μM forskolin and 100 μM IBMX (FSK/I) in the presence of vehicle (DMSO, 96 hrs) or PLX4720 (2 μM), AZD6244 (0.2 μM), PLX4720+AZD6244 (2 μM and 0.2 μM, respectively) or VRT11E (2 μM) for 96 hrs. FIG. 14A(e) Western blot analysis of MITF, vinculin (VINC) and the MITF target genes SILVER (SLV), tyrosinase related protein 1 (TRP1) and Melan-A (MelA) in immortalized, primary melanocytes grown in complete, cAMP-containing growth media (TICVA) or in the presence (+cAMP) or absence (cAMP-starved) of dbcAMP (1 mM) and IBMX (100 μM) for 96 hours. In parallel, cAMP-starved melanocytes were treated with vehicle control (DMSO) or stimulated with 10 μM forskolin and 100 μM IBMX (FSK/I), 1 mM dbcAMP and 100 μM IBMX (cAMP/I) or 1 μM α-melanocyte stimulating hormone (αMSH/I) for the indicated times. FIG. 14B(f) Melanin content of immortalized, primary melanocytes cultured for 96 hours in complete cAMP-containing growth media (TICVA) or basal growth media devoid of cAMP (−cAMP). FIG. 14B(g) Western blot analysis of MITF, phosphorylated ERK (Thr202/Tyr204, pERK), ERK and vinculin (VINC) in WM266.4 48 hours after viral expression of the indicated genes or treatment with 10 μM forskolin and 100 μM IBMX (FSK/I) in the presence of vehicle (DMSO, 96 hrs) or AZD6244 (200 nM, 96 hrs).

FIG. 15 shows western blot analysis of CREB phosphorylation (Ser133, pCREB), ERK phosphorylation (Thr202/Tyr204, pERK) and total CREB, ERK and vinculin (VINC) in WM266.4 treated with 200 nM AZD6244 for 96 hours, followed by pre-treatment for 1 hour with DMSO or 10 μM H89 and subsequent stimulation with forskolin (10 μM) and IBMX (100 μM) (FSK/I) for the indicated times.

FIG. 16 shows that combined treatment with MAPK-pathway inhibitors and histone deacetylase inhibitors suppressed cAMP mediated MITF expression and resistance (A) Western blot analysis of MITF, phosphorylated ERK (Thr202/Tyr204, pERK), total ERK and vinculin (VINC) in lysates extracted from human BRAFV600E positive melanoma biopsies. Time of biopsies are indicated: pre-initiation of treatment (P), following 10-14 days of MAPK-inhibitor treatment (on-treatment, O) or following relapse (R). (B) Western blot analysis of MITF, SOX10, acetylated histone H3 (Ac-H3), phosphorylated ERK (Thr202/Tyr204, pERK), total ERK and vinculin (VINC) in WM266.4. Cells were treated with DMSO or AZD6244 (200 nM) for 96 hours, followed by treatment with Panobinostat, Vorinostat or Entinostat for 18 hours at the indicated concentration and subsequently stimulated with 10 μM forskolin and 100 μM IBMX (FSK/I) for 6 hrs. (C) Western blot analysis of MITF, SOX10, acetylated histone H3 (Ac-H3), phosphorylated ERK (Thr202/Tyr204, pERK), total ERK and vinculin (VINC) in WM266.4, SKMEL19 and SKMEL28. Cells were treated as in (C), using 1 μM Panobinostat, 10 μM Saha and 30 μM Entinostat. (D) Cellular viability of WM266.4 treated with the indicated combinations of vehicle (DMSO), PLX4720 (2 μM), AZD6244 (0.2 μM), PLX4720+AZD6244 (2 μM and 0.2 μM, respectively), VRT11E (2 μM), Panobinostat, Vorinostat, Entinostat, 10 μM forskolin and 100 μM IBMX (FSK/I) at the indicated concentrations for 96 hrs. The bars for each treatment from left to right are DMSO, Panobinostat (10 nM), Vorinostat (1.5 μM), and Entinostat (1.5 μM). Cell viability is shown as a percent of DMSO in un-stimulated/non drug-treated cells. Error bars represent SD, n=6.

### DETAILED DESCRIPTION OF INVENTION

The present invention relates to the development of resistance to therapeutic agents used in the treatment of cancer and identification of targets that confer such resistance. The present invention also relates to identification of drug targets for facilitating an effective long-term treatment strategy and to identification of patients who would benefit from such treatment.

More specifically, the invention further relates to identifying the molecular basis of resistance to MAPK pathway inhibitors such as but not limited to RAF inhibitors, MEK inhibitors and ERK inhibitors, predicting or diagnosing such resistance prior to its manifestation, and overcoming such resistance.

As discussed in greater detail herein, the invention is premised in part on the finding that increased levels or activities of several particular markers, including guanine nucleotide exchange factors (GEFs), G protein coupled receptors (GPCRs), transcription factors, serine/threonine kinases, ubiquitin machinery proteins, adaptor proteins, protein tyrosine kinases, receptor tyrosine kinases, protein binding proteins, cytoskeletal proteins, and RNA binding proteins can confer such resistance. Accordingly, various aspects of the invention relate to measuring at least one such marker in a subject, including for example measuring a level or activity of one such marker, and diagnosing and/or treating a subject based on the level or activity of the marker.

Also as discussed in greater detail herein, the invention is premised in part on the finding that a GPCR cyclic AMP (cAMP)-dependent signaling pathway is associated with MAPK pathway inhibitor resistance. As further discussed herein, transcription factors downstream of cAMP and protein kinase A (PKA) in this GPCR pathway were found to be associated with MAPK pathway inhibitor resistance. These transcription factors included FOS, NR4A1, NR4A2, and MITF, as well as CREB1/AFT1. Accordingly, various aspects of the invention relate to measuring a (i.e., at least one) marker selected from (1) a GPCR that activates production of cAMP, (2) a GPCR pathway component selected from FOS, NR4A1, NR4A2, and MITF, and (3) a PKA-activated transcription factor that activates FOS, NR4A1, NR4A2, and MITF, in a subject, including for example measuring a level or activity of the marker, and diagnosing and/or treating a subject based on the level of the marker.

Also as discussed in greater detail herein, the invention is premised in part on the finding that contacting MAPK pathway inhibitor resistant cells with a histone deacetylase (HDAC) inhibitor restored sensitivity to MAPK pathway inhibitors. Accordingly, various aspects of the invention relate to treating a subject that is resistant to a MAPK pathway inhibitor (including for example a subject so identified based on the level or activity of one of the foregoing markers described herein) and/or treating a subject with an HDAC inhibitor together with a MAPK pathway inhibitor.

The mitogen-activated protein kinase (MAPK) cascade is a critical intracellular signaling pathway that regulates signal transduction in response to diverse extracellular stimuli, including growth factors, cytokines, and proto-oncogenes. Activation of this pathway results in transcription factor activation and alterations in gene expression, which ultimately lead to changes in cellular functions including cell proliferation, cell cycle regulation, cell survival, angiogenesis and cell migration. Classical MAPK signaling is initiated by receptor tyrosine kinases at the cell surface, however many other cell surface molecules are capable of activating the MAPK cascade, including integrins, heterotrimeric G-proteins, and cytokine receptors.

Ligand binding to a cell surface receptor, e.g., a receptor tyrosine kinase, typically results in phosphorylation of the receptor. The adaptor protein Grb2 associates with the phosphorylated intracellular domain of the activated receptor, and this association recruits guanine nucleotide exchange factors (GEFs) including SOS-I and CDC25 to the cell membrane. These particular GEFs interact with and activate the GTPase Ras. Common Ras isoforms include K-Ras, N-Ras, H-Ras and others. Following Ras activation, the serine/threonine kinase Raf (e.g., A-Raf, B-Raf or Raf-1) is recruited to the cell membrane through interaction with Ras. Raf is then phosphorylated. Raf directly activates MEKl and MEK2 by phosphorylation of two serine residues at positions 217 and 221. Following activation, MEKl and MEK2 phosphorylate tyrosine (Tyr-185) and threonine (Thr-183) residues in serine/threonine kinases Erkl and Erk2, resulting in Erk activation. Activated Erk regulates many targets in the cytosol and also translocates to the nucleus, where it phosphorylates a number of transcription factors regulating gene expression. Erk kinase has numerous targets, including Elk-l, c-Etsl, c-Ets2, p90RSKl, MNKl, MNK2, MSKl, MSK2 and TOB. While the foregoing pathway is a classical representation of MAPK signaling, there is considerable cross talk between the MAPK pathway and other signaling cascades.

Aberrations in MAPK signaling have a significant role in cancer biology. Altered expression of Ras is common in many cancers, and activating mutations in Ras have also been identified. Such mutations are found in up to 30% of all cancers, and are especially common in pancreatic (90%) and colon (50%) carcinomas. In addition, activating Raf mutations have been identified in melanoma and ovarian cancer. The most common mutation, BRAF^{V600E}, results in constitutive activation of the downstream MAP kinase pathway and is required for melanoma cell proliferation, soft agar growth, and tumor xenograft formation. Based on these observations, certain MAPK pathway inhibitors have been targeted in various cancer therapies. However, it has also been observed that certain patients have or develop a resistance to certain of these therapies.

The invention is based in part on the identification of targets that increase the likelihood of resistance, including those that confer resistance, to these therapies. Based on these findings, the invention provides methods that use the identified targets as diagnostic, theranostic and/or prognostic markers and as treatment targets in subjects having or likely to develop resistance. These various methods are described herein in greater detail.

Diagnostic, prognostic, and theranostic assays of the invention involve assaying gene copy, mRNA expression, protein expression and/or activity of one or more markers as described herein. The art is familiar with assays for copy number, mRNA expression levels, protein expression levels, and activity levels of the one or more markers as described herein. Non-limiting examples of such assays are described herein.

### Identification of Markers of MAPK Inhibitor Resistance

Several markers were identified as mediators of drug resistance through a high throughput functional screening assay. Generally, the high throughout functional screening assay identifies targets capable of driving resistance to clinically efficacious therapies such as MAPK pathway inhibitors such as RAF, MEK and ERK inhibitors. The assay is an open reading frame (ORF)-based functional screen for proteins that drive resistance to these therapeutic agents. The assay comprises use of a plurality of ORFs, such as 5,000, 10,000, 15,000 or more ORFs. The method may include providing a cell line having a known oncogenic mutation such as a RAF mutation (e.g., V600E RAF mutation). Examples of such cell lines include A375, G361, WM983b, WM266.4, WM88, UACC62, SKMEL28, and SKMEL19. A library of ORFS may be individually expressed in the cell line so that a plurality of clones, each expressing a different ORF from the library, may be further evaluated. In some embodiments, the plurality of clones is 10,000, 20,000, 30,000, 40,000, 50,000, 60,000, 70,000, 80,000, 90,000, 100,000, 125,000, 150,000, 200,000 or more clones. Each clone may be (1) exposed to a known inhibitor of the cell line and (2) monitored for growth changes based on the expression of the ORF. Any clones having a growth effect from the ORF expression alone, whether positive or negative, are eliminated. The remaining clones each expressing a different protein are then compared for viability between a control and a treated clone and normalized to a positive control. Increased cell viability after treatment with the inhibitor identifies ORFS that confer resistance. These ORFS are referred to herein as markers of resistance (or generally as markers).

Accordingly, aspects of the invention relate to a method of identifying a marker that confers resistance to a MAPK pathway inhibitor. The method generally comprises culturing cells having sensitivity to a MAPK pathway inhibitor, expressing a plurality of ORF clones in the cell cultures, each cell culture expressing a different ORF clone, exposing each cell culture to the MAPK pathway inhibitor, and identifying cell cultures having greater viability than a control cell culture after exposure to the MAPK pathway inhibitor to identify one or more ORF clones that confers resistance to the MAPK pathway inhibitor. In some embodiments, the cultured cells may have sensitivity to a RAF inhibitor, a MEK inhibitor, and/or an ERK inhibitor.

Any type of expression vector known to one skilled in the art may be used to express the ORF collection. By way of non-limiting example, a selectable, epitope-tagged, lentiviral expression vector capable of producing high titer virus and robust ORF expression in mammalian cells may be used to express the kinase collection (pLX-BLAST-V5).

To identify proteins capable of circumventing MAPK pathway inhibition, the arrayed ORF collection may be stably expressed in A375, G361, WM983b, WM266.4, WM88, UACC62, SKMEL28, and/or SKMEL19 cells, which are known to have sensitivity to MAPK pathway inhibitors, such as RAF inhibitor PLX4720, MEK inhibitor AZD6244, and ERK inhibitor VTX11e. Clones of ORF expressing cells treated with 1 μM PLX4720, AZD6244, VTX11e, or a combination of PLX4720 and AZD6244 are screened for viability relative to untreated cells and normalized to an assay-specific positive control, MEK1^{S218/222D} (MEK1^{DD}). ORFS that affected baseline viability or proliferation are removed from the analysis. Clones scoring above 2.5 standard deviations from the normalized mean may be further evaluated to identify a resistance conferring protein.

In other embodiments, the ORF collection may be stably expressed in a cell line having a different mutation in B-RAF, for example, another mutation at about amino acid position 600 such as V600K, V600D, and V600R. Additional B-RAF mutations include the mutations described in Davies et al. Nature, 417, 949-954, 2002, see Table 5, the specific teachings of which are incorporated by reference herein. In some embodiments, the ORF collection may be stably expressed in a cell line having sensitivity to other RAF kinase inhibitors including, but not limited to, PLX4032; GDC-0879; RAF265; sorafenib; SB590855 and/or ZM 336372. By way of non-limiting example, exemplary RAF inhibitors are shown in Table 6 and thereafter.

In some embodiments, the ORF collection may be stably expressed in a cell line having a sensitivity to a MEK inhibitor. Non-limiting examples of MEK inhibitors include, AZD6244; CI-1040; PD184352; PD318088, PD98059, PD334581, RDEA119, 6-Methoxy-7-(3-morpholin-4-yl-propoxy)-4-(4-phenoxy-phenylamino)-quinoline-3-carbonitrile and 4-[3-Chloro-4-(1-methyl-1H-imidazol-2-ylsulfanyl)-phenylamino]-6-methoxy-7-(3-morpholin-4-yl-propoxy)-quinoline-3-carbonitrile. Additional RAF and MEK inhibitors are described below. By way of non-limiting example, exemplary MEK inhibitors are shown in Table 7 and thereafter.

In some embodiments, the ORF collection may be stably expressed in a cell line having sensitivity to other MAPK pathway inhibitors including, but not limited to, those shown in Tables 6-8.

More specifically, the assay used to identify markers of MAPK pathway inhibitor resistance involved individually transfecting a large number of ORFS into a cell line that was otherwise susceptible to MAPK pathway inhibitors such as RAF inhibitor PLX4720 and MEK inhibitor AZD6244, thereby creating clones of the lines, each expressing one ORF from the screen. The clones were then cultured in the presence of RAF inhibitor PLX4720 alone, MEK inhibitor AZD6244 alone, PLX4720 and AZD6244 together, or ERK inhibitor VTX-11E. The major readouts were cell viability and proliferation in the presence of inhibitor. An increase in viability and/or proliferation in the presence of the inhibitor as compared with a clone transfected with a negative control ORF (e.g., a non-human gene ORF such as LacZ or eGFP) is indicative of a protein that confers drug resistance. The protein is then further identified as a predictive or diagnostic marker and a target for therapy.

### Markers

As described herein, a large-scale ORF screen involving the use of several melanoma cell lines was used to identify markers of resistance to a MAPK pathway inhibitor. It was found that overexpression of certain markers in cells that are otherwise susceptible to MAPK pathway inhibitors rendered the cells resistant to such inhibitors. These markers included guanine nucleotide exchange factors (GEFs), G protein coupled receptors (GPCRs), transcription factors, serine/threonine kinases, ubiquitin machinery proteins, adaptor proteins, protein tyrosine kinases, receptor tyrosine kinases, protein binding proteins, cytoskeletal proteins, and RNA binding proteins. This unexpected finding indicates that resistance to MAPK pathway inhibitors may be predicted based on a particular marker in a subject or in cancer cells from the subject. The markers identified in the large-scale ORF screen are provided in Table 1.

TABLE 1
Markers

| | NCBI | | |
|---|---|---|---|
| | Entrez | | |
| Gene | Human | | |
| Symbol | Gene ID | Transcript IDs | Gene Class |
| POU5F1 | 5460 | NM_002701.4, | Transcription Factor |
| | | NM_001173531.1, | |
| | | NM_203289.4 | |
| HOXD9 | 3235 | NM_014213.3 | Transcription Factor |
| EBF1 | 1879 | NM_024007.3 | Transcription Factor |
| HNF4A | 3172 | NM_000457.4 | Transcription Factor |
| | | NM_001030003.2 | |
| | | NM_001030004.2 | |
| | | NM_001258355.1 | |
| | | NM_175914.4 | |
| | | NM_178849.2 | |
| | | NM_178850.2 | |
| SP6 | 80320 | NM_001258248.1 | Transcription Factor |
| | | NM_199262.2 | |
| ESRRG | 2104 | NM_001134285.2 | Transcription Factor |
| | | NM_001243505.1 | |
| | | NM_001243506.1 | |
| | | NM_001243507.1 | |
| | | NM_001243509.1 | |
| | | NM_001243510.1 | |
| | | NM_001243511.1 | |
| | | NM_001243512.1 | |
| | | NM_001243513.1 | |
| | | NM_001243514.1 | |
| | | NM_001243515.1 | |
| | | NM_001243518.1 | |
| | | NM_001243519.1 | |
| | | NM_001438.3 | |
| | | NM_206594.2 | |
| | | NM_206595.2 | |
| TFEB | 7942 | NM_001167827.2 | Transcription Factor |
| | | NM_001271943.1 | |
| | | NM_001271944.1 | |
| | | NM_001271945.1 | |
| | | NM_007162.2 | |
| FOXA3 | 3171 | NM_004497.2 | Transcription Factor |
| FOX | 23543 | NM_001031695.2 | Transcription Factor |
| | | NM_001082576.1 | |
| | | NM_001082577.1 | |
| | | NM_001082578.1 | |
| | | NM_001082579.1 | |
| | | NM_014309 | |
| MITF | 4286 | NM_000248.3 | Transcription Factor |
| | | NM_001184967.1 | |
| | | NM_001184968.1 | |
| | | NM_006722.2 | |
| | | NM_198158.2 | |
| | | NM_198159.2 | |
| | | NM_198177.2 | |
| | | NM_198178.2 | |
| FOXJ1 | 2302 | NM_001454.3 | Transcription Factor |
| XBP1 | 7494 | NM_005080.3 | Transcription Factor |
| | | NM_001079539.1 | |
| NR4A1 | 3164 | NM_001202233.1 | Transcription Factor |
| | | NM_002135.4 | |
| | | NM_173157.2 | |
| ETV1 | 2115 | NM_001163147.1 | Transcription Factor |
| | | NM_001163148.1 | |
| | | NM_001163149.1 | |
| | | NM_001163150.1 | |
| | | NM_001163151.1 | |
| | | NM_001163152.1 | |
| | | NM_004956.4 | |
| HEY1 | 23462 | NM_001040708.1 | Transcription Factor |
| | | NM_012258.3 | |
| KLF6 | 1316 | NM_001160124.1 | Transcription Factor |
| | | NM_001160125.1 | |
| | | NM_001300.5 | |
| HEY2 | 23493 | NM_012259.2 | Transcription Factor |
| JUNB | 3726 | NM_002229.2 | Transcription Factor |
| SP8 | 221833 | NM_182700.4 | Transcription Factor |
| | | NM_198956.2 | |
| OLIG3 | 167826 | NM_175747.2 | Transcription Factor |
| PURG | 29942 | NM_001015508.1 | Transcription Factor |
| | | NM_013357.2 | |
| FOXP2 | 93986 | NM_001172766.2 | Transcription Factor |
| | | NM_001172767.2 | |
| | | NM_014491.3 | |
| | | NM_148898.3 | |
| | | NM_148899.3 | |
| | | NM_148900.3 | |
| YAP1 | 10413 | NM_001130145.2 | Transcription Factor |
| | | NM_001195044.1 | |
| | | NM_001195045.1 | |
| | | NM_006106.4 | |
| NFE2L1 | 4779 | NM_003204.2 | Transcription Factor |
| TLE1 | 7088 | NM_005077.3 | Transcription Factor |
| PASD1 | 139135 | NM_173493.2 | Transcription Factor |
| TP53 | 7157 | NM_000546.5 | Transcription Factor |
| | | NM_001126112.2 | |
| | | NM_001126113.2 | |
| | | NM_001126114.2 | |
| | | NM_001126115.1 | |
| | | NM_001126116.1 | |
| | | NM_001126117.1 | |
| | | NM_001126118.1 | |
| | | NM_001276695.1 | |
| | | NM_001276696.1 | |
| | | NM_001276697.1 | |
| | | NM_001276698.1 | |
| | | NM_001276699.1 | |
| | | NM_001276760.1 | |
| | | NM_001276761.1 | |
| WWTR1 | 25937 | NM_001168278.1 | Transcription Factor |
| | | NM_001168280.1 | |
| | | NM_015472.4 | |
| SATB2 | 23314 | NM_001172509.1 | Transcription Factor |
| | | NM_001172517.1 | |
| | | NM_015265.3 | |
| NR4A2 | 4929 | NM_006186.3 | Transcription Factor |
| HAND2 | 9464 | NM_021973.2 | Transcription Factor |
| GCM2 | 9247 | NM_004752.3 | Transcription Factor |
| SHOX2 | 6474 | NM_001163678.1 | Transcription Factor |
| | | NM_003030.4 | |
| | | NM_006884.3 | |
| NANOG | 79923 | NM_024865.2 | Transcription Factor |
| CRX | 1406 | NM_000554.4 | Transcription Factor |
| ZNF423 | 23090 | NM_001271620.1 | Transcription Factor |
| | | NM_015069.3 | |
| ISX | 91464 | NM_001008494.1 | Transcription Factor |
| ETS2 | 2114 | NM_001256295.1 | Transcription Factor |
| | | NM_005239.5 | |
| SIM2 | 6493 | NM_005069.3 | Transcription Factor |
| | | NM_009586.2 | |
| MAFB | 9935 | NM_005461.3 | Transcription Factor |
| MY0D1 | 4654, | NM_002478.4 | Transcription Factor |
| HOXC11 | 3227 | NM_014212.3 | Transcription Factor |
| GPR4 | 2828 | NM_005282.2 | GPCR |
| GPR3 | 2827 | NM_005281.3 | GPCR |
| GPBAR1 | 151306 | NM_001077191.1 | GPCR |
| | | NM_001077194.1 | |
| | | NM_170699.2 | |
| HTR2C | 3358 | NM_000868.2 | GPCR |
| | | NM_001256760.1 | |
| | | NM_001256761.1 | |
| MAS1 | 4142 | NM_002377.2 | GPCR |
| ADORA2A | 135 | NM_000675.4 | GPCR |
| GPR161 | 23432 | NM_001267609.1 | GPCR |
| | | NM_001267610.1 | |
| | | NM_001267611.1 | |
| | | NM_001267612.1 | |
| | | NM_001267613.1 | |
| | | NM_001267614.1 | |
| | | NM_153832.2 | |
| GPR119 | 139760 | NM_178471.2 | GPCR |
| LPAR4 | 2846 | NM_005296.2 | GPCR |
| GPR132 | 29933 | NM_013345.2 | GPCR |
| LPAR1 | 1902 | NM_001401.3 | GPCR |
| | | NM_057159.2 | |
| GPR35 | 2859 | NM_001195381.1 | GPCR |
| | | NM_001195382.1 | |
| P2RY8 | 286530 | NM_178129.4 | GPCR |
| VAV1 | 7409 | NM_001258206.1 | GTP-GEF |
| | | NM_001258207.1 | |
| | | NM_005428.3 | |
| ARHGEF3 | 50650 | NM_001128615.1 | GTP-GEF |
| | | NM_001128616.1 | |
| | | NM_019555.2 | |
| RASGRP2 | 10235 | NM_001098670.1 | GTP-GEF |
| | | NM_001098671.1 | |
| | | NM_153819.1 | |
| RASGRP3 | 25780 | NM_001139488.1 | GTP-GEF |
| | | NM_015376.2 | |
| | | NM_170672.2 | |
| ARHGEF9 | 23229 | NM_001173479.1 | GTP-GEF |
| | | NM_001173480.1 | |
| | | NM_015185.2 | |
| RASGRP4 | 115727 | NM_001146202.1 | GTP-GEF |
| | | NM_001146203.1 | |
| | | NM_001146204.1 | |
| | | NM_001146205.1 | |
| | | NM_001146206.1 | |
| | | NM_001146207.1 | |
| | | NM_170604.2 | |
| SPATA13 | 221178 | NM_001166271.1 | GTP-GEF |
| | | NM_153023.2 | |
| PLEKHG6 | 55200 | NM_001144856.1 | GTP-GEF |
| | | NM_001144857.1 | |
| | | NM_018173.3 | |
| MCF2L | 23263 | NM_001112732.2 | GTP-GEF |
| | | NM_024979.4 | |
| PLEKHG5 | 57449 | NM_001042663.1 | GTP-GEF |
| | | NM_001042664.1 | |
| | | NM_001042665.1 | |
| | | NM_001265592.1 | |
| | | NM_001265593.1 | |
| | | NM_001265594.1 | |
| | | NM_020631.4 | |
| | | NM_198681.3 | |
| NGEF | 25791 | NM_001114090.1 | GTP-GEF |
| | | NM_019850.2 | |
| PRKACA | 5566 | NM_002730.3 | Serine/Theonine Kinase |
| | | NM_207518.1 | |
| RAF1 | 5894 | NM_002880.3 | Serine/Theonine Kinase |
| NF2 | 4771 | NM_000268.3 | Serine/Theonine Kinase |
| | | NM_016418.5 | |
| | | NM_181825.2 | |
| | | NM_181828.2 | |
| | | NM_181829.2 | |
| | | NM_181830.2 | |
| | | NM_181831.2 | |
| | | NM_181832.2 | |
| | | NM_181833.2 | |
| PRKCE | 5581 | NM_005400.2 | Serine/Theonine Kinase |
| PAK3 | 5063 | NM_001128166.1 | Serine/Theonine Kinase |
| | | NM_001128167.1 | |
| | | NM_001128168.1 | |
| | | NM_001128172.1 | |
| | | NM_001128173.1 | |
| | | NM_002578.3 | |
| MOS | 342 | NM_005372.1 | Serine/Theonine Kinase |
| FBXO5 | 26271 | NM_001142522.1 | Ubiquitin Machinery |
| | | NM_012177.3 | |
| TNFAIP1 | 7126 | NM_021137.4 | Ubiquitin Machinery |
| KLHL10 | 317719 | NM_152467.3 | Ubiquitin Machinery |
| ARIH1 | 25820 | NM_005744.3 | Ubiquitin Machinery |
| TRIM50 | 135892 | NM_178125.2 | Ubiquitin Machinery |
| CRKL | 1399 | NM_005207.3 | Adapter Prot. |
| CRK | 1398 | NM_005206.4 | Adapter Prot. |
| | | NM_016823.3 | |
| TRAF3IP2 | 10758 | NM_001164281.2 | Adapter Prot. |
| | | NM_001164283.2 | |
| | | NM_147686.3 | |
| FRS3 | 10817 | NM_006653.3 | Adapter Prot. |
| SQSTM1 | 8878 | NM_001142298.1 | Adapter Prot. |
| | | NM_001142299.1 | |
| | | NM_003900.4 | |
| HCK | 3055 | NM_001172129.1 | Protein Tyrosine Kinase |
| | | NM_001172130.1 | |
| | | NM_001172131.1 | |
| | | NM_001172132.1 | |
| | | NM_001172133.1 | |
| | | NM_002110.3 | |
| BTK | 695 | NM_000061.2 | Protein Tyrosine Kinase |
| LCK | 3932 | NM_001042771.1 | Protein Tyrosine Kinase |
| | | NM_005356.3 | |
| SRC | 6714 | NM_005417.3 | Protein Tyrosine Kinase |
| | | NM_198291.1 | |
| LYN | 4067 | NM_001111097.2 | Protein Tyrosine Kinase |
| | | NM_001111097.2 | |
| FGR | 2268 | NM_001042729.1 | Receptor Tyrosine |
| | | NM_001042747.1 | Kinase |
| | | NM_005248.2 | |
| FGFR2 | 2263 | NM_000141.4 | Receptor Tyrosine |
| | | NM_001144913.1 | Kinase |
| | | NM_001144914.1 | |
| | | NM_001144915.1 | |
| | | NM_001144916.1 | |
| | | NM_001144917.1 | |
| | | NM_001144918.1 | |
| | | NM_001144919.1 | |
| | | NM_022970.3 | |
| | | NM_023029.2 | |
| AXL | 558 | NM_001699.4 | Receptor Tyrosine |
| | | NM_021913.3 | Kinase |
| TYRO3 | 7301 | NM_006293.3 | Receptor Tyrosine |
| | | | Kinase |
| CARD9 | 64170 | NM_052813.4 | Protein Binding |
| | | NM_052814.3 | |
| WDR5 | 11091 | NM_017588.2 | Protein Binding |
| | | NM_052821.3 | |
| PVRL1 | 5818 | NM_002855.4 | Cytoskeletal |
| | | NM_203285.1 | |
| | | NM_203286.1 | |
| TEKT5 | 46279 | NM_144674.1 | Cytoskeletal |
| SAMD4B | 55095 | NM_018028.2 | RNA-Binding Protein |
| SAMD4A | 23034 | NM_001161576.2 | RNA-Binding Protein |
| | | NM_001161577.1 | |
| | | NM_015589.5 | |
| VPS28 | 51160 | NM_016208.2 | — |
| | | NM_183057.1 | |
| IFNA10 | 3446 | NM_002171.1 | — |
| KLHL34 | 257240 | NM_153270.1 | — |
| TNFRSF13B | 23495 | NM_012452.2 | — |
| CYP2E1 | 1571 | NM_000773.3 | — |
| BRMS1L | 84312 | NM_032352.3 | — |
| ADAP2 | 55803 | NM_018404.2 | — |
| MLYCD | 23417 | NM_012213.2 | — |
| MAGEA9 | 4108 | NM_005365.4 | — |
| RIT2 | 6014 | NM_001272077.1 | — |
| | | NM_002930.3 | |
| KCTD1 | 84252 | NM_001136205.2 | — |
| | | NM_001142730.2 | |
| | | NM_001258221.1 | |
| | | NM_001258222.1 | |
| | | NM_198991.3 | |

Diagnostic, prognostic, and theranostic assays of the invention involve assaying gene copy, mRNA expression, protein expression and/or activity of one or more markers. The art is familiar with assays for copy number, mRNA expression levels, protein expression levels, and activity levels of the one or more markers (see, e.g., Sambrook, Fritsch and Maniatis, MOLECULAR CLONING: A LABORATORY MANUAL, (Current Edition); CURRENT PROTOCOLS IN MOLECULAR BIOLOGY (F. M. Ausubel et al. eds., (Current Edition)); the series METHODS IN ENZYMOLOGY (Academic Press, Inc.): PCR 2: A PRACTICAL APPROACH (Current Edition) ANTIBODIES, A LABORATORY MANUAL and ANIMAL CELL CULTURE (R. I. Freshney, ed. (1987)). DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); Fundamental Virology, 2nd Edition, vol. I & II (B. N. Fields and D. M. Knipe, eds.).

Copy number can be measured, for example, using sequencing, fluorescence in situ hybridization (FISH) or a Southern blot. mRNA expression levels may be measured, for example, using Northern analysis or quantitative RT-PCR (qPCR). Protein expression levels may be measured, for example, using Western immunoblotting analysis or immunohistochemistry.

Methods for measuring a marker activity are also known in the art and commercially available (see, e.g., enzyme and protein activity assays from Invitrogen, Piercenet, AbCam, EMD Millipore, or SigmaAldrich). Non-limiting examples of assays for measuring marker activity include western blot, enzyme-linked immunosorbent assay (ELISA), fluorescent activated cell sorting (FACS), luciferase or chloramphenicol acetyl transferase reporter assay, protease colorimetric assay, immunoprecipitation (including Chromatin-IP), PCR, qPCR, or fluorescence resonance energy transfer.

Non-limiting examples of marker activities include phosphorylation (kinase or phosphotase activity), ubiquitination, SUMOylation, Neddylation, cytoplasmic or nuclear localization, binding to a binding partner (such as a protein, DNA, RNA, ATP, or GTP), transcription, translation, post-translation modification (such as glycosylation, methylation, or acetylation), chromatin modification, proteolysis, receptor activation or inhibition, cyclic AMP activation or inactivation, GTPase activation or inactivation, electron transfer, hydrolysis, or oxidation.

Marker activity may be measured indirectly. For example, if a marker must be phosphorylated or dephosphorylated before becoming active, a phosphorylation level of the marker may indicate an activity level.

In some embodiments, the methods described herein comprise comparing the gene copy number, mRNA or protein level, or activity level of the marker in the cancer cells with a gene copy number, mRNA or protein level, or activity level of the marker in normal cells, and

In some embodiments, the methods described herein comprise identifying a subject having cancer cells with increased gene copy number, mRNA or protein level, or activity level of the marker relative to normal cells as a subject who is at risk of developing resistance to a MAPK pathway inhibitor.

GPCR cAMP-Dependent Pathway

As described herein, the invention is premised in part on the finding that a GPCR cyclic AMP(cAMP)-dependent signaling pathway is associated with MAPK pathway inhibitor resistance. GPCRs that activate cAMP, as well as transcription factors downstream of cAMP and protein kinase A (PKA) in this GPCR pathway were found to be associated with MAPK pathway inhibitor resistance. Such transcription factors included FOS, NR4A1, NR4A2, and MITF, and PKA-activated transcription factors.

Accordingly, various aspects of the invention relate to measuring a marker selected from a GPCR that activates production of cAMP, a GPCR pathway component selected from FOS, NR4A1, NR4A2, and MITF, and a PKA-activated transcription factor that activates FOS, NR4A1, NR4A2, and MITF, in a subject, including for example measuring a level or activity of the marker, and diagnosing and/or treating a subject based on the level of the marker.

A GPCR that activates production of cAMP can be identified, for example, by measuring a level of cAMP using an assay such as ELISA or a cAMP-Glo™ Assay (Promega) after activation or overexpression of the GPCR in a cell. If the level of cAMP is elevated, this indicates that the GPCR is capable of activating production of cAMP. In some embodiments, a GPCR that activates production of cyclic AMP is GPR4, GPR3, GPBAR1, HTR2C, MAS1, ADORA2A, GPR161, GPR52, GPR101, or GPR119.

A PKA-activated transcription factor that activates FOS, NR4A1, NR4A2, and MITF can be identified, for example, by measuring a level of FOS, NR4A1, NR4A2, and MITF after activation or overexpression of the PKA-activated transcription factor. A level of FOS, NR4A1, NR4A2, and MITF can be measured using an assay such as quantitative PCR or a western blot. If the level of FOS, NR4A1, NR4A2, and MITF is elevated, this indicates that the PKA-activated transcription factor is capable of activating FOS, NR4A1, NR4A2, and MITF. In some embodiments, the PKA-activated transcription factor that activates FOS, NR4A1, NR4A2, and MITF is CREB1, ATF4, ATF1, CREB3, CREB5, CREB3L1, CREB3L2, CREB3L3, or CREB3L4.

The markers selected from a GPCR that activates production of cAMP and a GPCR pathway component selected from FOS, NR4A1, NR4A2, and MITF, and a PKA-activated transcription factor that activates FOS, NR4A1, NR4A2, and MITF are provided in Tables 2-4.

TABLE 2
Exemplary GPCRs that activate production of cyclic AMP

| | NCBI Entrez | |
|---|---|---|
| Gene Symbol | Human Gene ID | Transcript IDs |
| GPR4 | 2828 | NM_005282.2 |
| GPR3 | 2827 | NM_005281.3 |
| GPBAR1 | 151306 | NM_001077191.1 |
| | | NM_001077194.1 |
| | | NM_170699.2 |
| HTR2C | 3358 | NM_000868.2 |
| | | NM_001256760.1 |
| | | NM_001256761.1 |
| MAS1 | 4142 | NM_002377.2 |
| ADORA2A | 135 | NM_000675.4 |
| GPR161 | 23432 | NM_001267609.1 |
| | | NM_001267610.1 |
| | | NM_001267611.1 |
| | | NM_001267612.1 |
| | | NM_001267613.1 |
| | | NM_001267614.1 |
| | | NM_153832.2 |
| GPR52 | 9293 | NM_005684.4 |
| GPR101 | 83550 | NM_054021.1 |
| GPR119 | 139760 | NM_178471.2 |

TABLE 3
Exemplary GPCR pathway components

| | NCBI Entrez | |
|---|---|---|
| Gene Symbol | Human Gene ID | Transcript IDs |
| FOS | 2353 | NM_005252.3 |
| NR4A1 | 3164 | NM_001202233.1 |
| | | NM_002135.4 |
| | | NM_173157.2 |
| NR4A2 | 4929 | NM_006186.3 |
| MITF | 4286 | NM_000248.3 |
| | | NM_001184967.1 |
| | | NM_001184968.1 |
| | | NM_006722.2 |
| | | NM_198158.2 |
| | | NM_198159.2 |
| | | NM_198177.2 |
| | | NM_198178.2 |

TABLE 4
Exemplary PKA-activated transcription factors that activate
FOS, NR4A1, NR4A2, and MITF

| | NCBI Entrez | |
|---|---|---|
| Gene Symbol | Human Gene ID | Transcript IDs |
| CREB1 | 1385 | NM_004379.3 |
| | | NM_134442.3 |
| ATF4 | 468 | NM_001675.2 |
| | | NM_182810.1 |
| ATF1 | 466 | NM_005171.4 |
| CREB3 | 10488 | NM_006368.4 |
| CREB5 | 9586 | NM_001011666.1 |
| | | NM_004904.2 |
| | | NM_182898.2 |
| | | NM_182899.3 |
| CREB3L1 | 90993 | NM_052854.2 |
| CREB3L2 | 64764 | NM_001253775.1 |
| | | NM_194071.3 |
| CREB3L3 | 84699 | NM_001271995.1 |
| | | NM_001271996.1 |
| | | NM_001271997.1 |
| | | NM_032607.2 |
| CREB3L4 | 148327 | NM_001255978.1 |
| | | NM_001255979.1 |
| | | NM_001255980.1 |
| | | NM_001255981.1 |
| | | NM_130898.3 |

Diagnostic, prognostic, and theranostic assays of the invention involve assaying gene copy, mRNA expression, protein expression and/or activity of one or more of these markers. Such assays are described herein.

Activity levels of a GPCR that activates production of cAMP can be measured using several different methods. For example, activity can be determined by measuring a level of cAMP using an assay such as ELISA or a cAMP-Glo™ Assay (Promega). In another example, activity can be determined by measuring a level of phosphorylation of a CREB family member such as CREB1, ATF4, ATF1, CREB3, CREB5, CREB3L1, CREB3L2, CREB3L3, or CREB3L4 using an assay such as a western blot. In another example, activity can be determined by measuring a level of FOS, NR4A1, NR4A2, or MITF using an assay such as quantitative PCR or a western blot. An elevated level of cAMP, phosphorylation of a CREB family member, or FOS, NR4A1, NR4A2, or MITF indicates elevated activity of the GPCR.

Activity levels of the transcription factors FOS, NR4A1, NR4A2, and MITF can be measured using several different methods. For example, activity can be determined by measuring binding of the transcription factors to DNA using an assay such as chromatin immunoprecipitation, where an increased level of binding to DNA indicates elevated activity. In another example, activity can be determined by measuring one or more transcriptional targets of FOS, NR4A1, NR4A2, and MITF using an assay such as quantitative PCR or a western blot, where an increased level of the one or more transcriptional targets may indicate elevated activity.

An activity level of a PKA-activated transcription factor that activates FOS, NR4A1, NR4A2, and MITF, such as CREB1, ATF4, ATF1, CREB3, CREB5, CREB3L1, CREB3L2, CREB3L3, and CREB3L4, can be measured using several different methods. For example, activity can be determined by measuring a level of phosphorylation of the PKA-activated transcription factor using an assay such as a western blot, where an increased level of phosphorylation indicates elevated activity. In another example, activity can be determined by measuring binding of the transcription factor to DNA using an assay such as chromatin immunoprecipitation, where an increased level of binding to DNA indicates elevated activity. In yet another example, activity can be determined by measuring one or more transcriptional targets of the transcription factor using an assay such as quantitative PCR or a western blot, where an increased level of the one or more transcriptional targets may indicate elevated activity.

Also as described herein, the invention is premised in part on the finding that activation of cAMP-mediated signaling through use of exogenous cAMP or the cAMP activator forskolin was sufficient to induce MAPK pathway inhibitor resistance. This induced MAPK pathway inhibitor resistance could be reversed through use of an HDAC inhibitor. Accordingly, in some embodiments, the methods described herein comprise identifying a subject having cancer cells with increased gene copy number, mRNA or protein level, or activity level of a marker selected from a GPCR that activates production of cAMP and a GPCR pathway component selected from FOS, NR4A1, NR4A2, and MITF, and a PKA-activated transcription factor that activates FOS, NR4A1, NR4A2, and MITF relative to normal cells as a subject (i) who is at risk of developing resistance to a MAPK pathway inhibitor, (ii) who is likely to benefit from treatment with an HDAC inhibitor, (iii) who is likely to benefit from treatment with a combination therapy comprising an HDAC inhibitor, and/or (iv) who is likely to benefit from treatment with a combination therapy comprising a MAPK pathway inhibitor and an HDAC inhibitor.

### GEFS

It has been found, in accordance with the invention, that overexpression of certain GEFs in cells that are otherwise susceptible to the MAPK pathway inhibitors renders the cells resistant to such inhibitors. This unexpected finding indicates that resistance to MAPK pathway inhibitors may be predicted based on a level of a marker of a subject or of cancer cells from the subject. The finding also indicates that therapy with one or more GEF inhibitors alone or in combination with other therapies, including for example one or more MAPK pathway inhibitors, may be used in subjects having or likely to develop resistance to a potential therapy or a therapy that the subject has received or is receiving.

“Guanine exchange factors” (or GEFs) as used herein describe a class of proteins that catalyze the release of GDP and thus allow the binding of GTP. GEFs include but are not limited to GEFs from Ras, Rac, Rho, and CDC42. GEFs include, but are not limited to, ARHGEF2, ARHGEF3, ARHGEF9, ARHGEF19, IQSEC1, MCF2L, NGEF, PLEKHG3, PLEKHG5, PLEKHG6, TBC1 D3G, SPATA13, and VAV1. These GEFs, their gene IDs, and aliases are provided in Table 1 and Table 5. As shown in the Table 5, GEFs may be characterized according to the GTPase for which they exhibit specificity. For example, GEFs may be Rho-specific GEFs (e.g., ARHGEF19), or Cdc42-specific GEFs (e.g., ARHGEF9). Other specificities are provided in Table 5.

Other examples of GEFs include Abr, AAH26778; AAH33666; AAH42606, Alsin, Asef, BAA91741; BAB15719/hClg; BAB 15765, BAB71009; BAC85128, Bcr, CDC25, CDEP/Farp1 Farp2/Frg, Dbs, Dbl, Duo, Duet, Ect2, Fgd2, Fgd1, Fgd3, Frabin, GEF-H1; GEF-T, hPEM-2; Intersectin, ITSN, Rani; Itan2; KIAA 0294, KIAA 0861; KIAA 1362; KIAA 1626; KIAA 1909. LARG, Lbc, Lfc, N-GEF/ephexin; Neuroblastoma, Net1, Obscurin, PDZ-RhoGEF, alpha-Pix, beta-Pix, RasGRF, RasGRF1; RasGRF2; P-Rex, P-Rex1; P-Rex2, p63 RhoGEF; p114-RhoGEF, p115-RhoGEF, p164-RhoGEF, p190-RhoGEF; Scambio; Sos, Sos1; Sos2; Sos1/2, S-GEF, Tiam1, Tiam2, Tim, Trio, Trio N; Trio C; Tuba, Vsm-RhoGEF, WGEF; Xpin, XP027307; XP085127; XP294019; XP376334, Vav1, Vav2, and Vav3. In some important embodiments, the GEF is VAV1 and the GEF inhibitor is a VAV1 inhibitor.

TABLE 5

| | | | | Prot. | |
|---|---|---|---|---|---|
| | NCBI | | | Match | |
| Symbol | Gene ID | Alias | Family | % | Pathway |
| ARHGEF9 | 23229 | hPEM-2/PEM2 | Dbl | 100 | Cdc42 |
| ARHGEF19 | 128272 | WGEF | Dbl | 100 | Rho |
| ARHGEF3 | 50650 | XPLN | Dbl | 100 | Rho |
| MCF2L | 23263 | DBG | Dbl | 100 | Rho |
| NGEF | 25791 | EPHEXIN | Dbl | 99 | Rho |
| VAV1 | 7409 | VAV | Dbl | 95 | Rho |
| ARHGEF2 | 9181 | GEF-H1 | Dbl | 99 | Rho/Rac |
| PLEKHG3 | 26030 | | Plekhg | 92 | Rho |
| PLEKHG5 | 57449 | | Plekhg | 94 | Rho |
| PLEKHG6 | 55200 | | Plekhg | 100 | Rho |
| IQSEC1 | 9922 | | | 99 | ARF |
| TBC1D3G | 654341 | | | 100 | Rab |
| SPATA13 | 221178 | | | 99 | Rho |

GEF activity may be measured, for example, by detecting nucleotide release and/or transfer. As an example, a high throughput fluorescence based nucleotide exchange assay can be used to identify compounds that inhibit the guanine nucleotide exchange cycle of a GTPase such as but not limited to the Ras superfamily GTPases. The assay capitalizes on spectroscopic differences between bound and unbound fluorescent nucleotide analogs to monitor guanine exchange. Fluorophore-conjugated nucleotides have a low quantum yield of fluorescence in solution due to intermolecular quenching by solvent and intramolecular quenching by the guanine base. However, upon binding to G-protein, the fluorescence emission intensity from the fluorophore is greatly enhanced. The fluorescence based nucleotide exchange assay can be used to identify compounds that act via different mechanisms, all of which directly impact the nature of guanine nucleotide exchange. In this manner, the assay allows for identification of compounds that can act on the guanine nucleotide exchange factors (GEF) and/or the GTPases.

Thus, a method of identifying compounds having the ability to modulate the guanine nucleotide exchange cycle of a GTPase may comprise: a) contacting the compound with a guanine nucleotide exchange factor and a GTPase and obtaining a baseline fluorescence measurement; b) contacting the guanine nucleotide exchange factor and the GTPase without the compound and obtaining a baseline fluorescence measurement; c) adding a fluorophore-conjugated GTP to the components of (a) and (b), respectively; d) obtaining fluorescence measurements of the respective components of (c) over time; e) subtracting the respective baseline fluorescence measurements of (a) and (b) from each fluorescence measurement of (d); and f) comparing the resulting fluorescence values of (e), wherein a decrease or increase in the rate of fluorescence change with the compound as compared with the rate of fluorescence change without the compound identifies a compound having the ability to modulate the guanine nucleotide exchange cycle of GTPases.

More detailed description of such GEF activity assays can be found in granted U.S. Pat. No. 7,807,400.

### Inhibitors

Aspects of the invention relate to uses of MAPK pathway inhibitors, HDAC inhibitors, and GEF inhibitors, and combinations thereof. MAPK inhibitors include RAF, MEK, and ERK inhibitors.

The inhibitor may target the gene, mRNA expression, protein expression, and/or activity, in all instances reducing the level and/or activity, in whole or in part, of the target of the inhibitor (e.g., GEF, HDAC, RAF, MEK, or ERK).

Non-limiting examples of RAF inhibitors include RAF265, sorafenib, dabrafenib (GSK2118436), SB590885, PLX 4720, PLX4032, GDC-0879 and/or ZM 336372. By way of non-limiting example, exemplary RAF inhibitors are shown in Table 6 and thereafter.

Non-limiting examples of MEK inhibitors include, AZD6244, CI-1040/PD184352, PD318088, PD98059, PD334581, RDEA119, 6-Methoxy-7-(3-morpholin-4-yl-propoxy)-4-(4-phenoxy-phenylamino)-quinoline-3-carbonitrile and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, trametinib (GSK1120212), and/or ARRY-438162. By way of non-limiting example, exemplary MEK inhibitors are shown in Table 7 and thereafter.

Non-limiting examples of ERK inhibitors include VTX11e, AEZS-131 (Aeterna Zentaris), PD98059, FR180204, and/or FR148083. By way of non-limiting example, exemplary MEK inhibitors are shown in Table 8 and thereafter.

In some embodiments, two MAPK pathway inhibitors may be used in combination, for example, wherein one of a first of the two MAPK inhibitors is a RAF inhibitor and a second of the two MAPK inhibitors is a MEK inhibitor. In some embodiments, the first inhibitor is dabrafenib and the second inhibitor is trametinib.

Examples of GEF inhibitors are described herein.

Non-limiting examples of HDAC inhibitors include Vorinostat, CI-994, Entinostat, BML-210, M344, NVP-LAQ824, Panobinostat, Mocetinostat, and Belinostat. By way of non-limiting example, exemplary HDAC inhibitors are shown in Table 9 and thereafter.

TABLE 6
Exemplary RAF Inhibitors

| | Name | CAS No. | Structure |
|---|---|---|---|
| 1 | RAF265 | 927880- 90-8 | 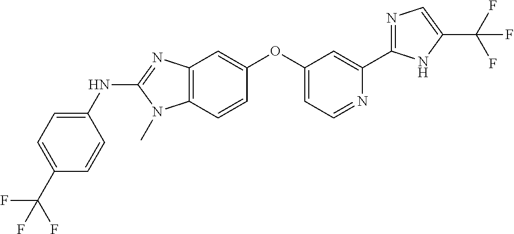 [SMILES: Cn1c(Nc2ccc(C(F)(F)F)cc2)nc2cc(Oc3ccnc(-c4ncc(C(F)(F)F)[nH]4)c3)ccc21] |
| 2 | Sorafenib Tosylate Nexavar Bay 43-9006 | 475207- 59-1 | 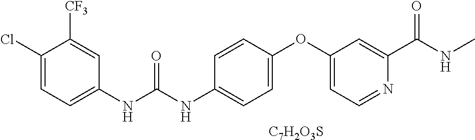 |
| 3 | Sorafenib 4-[4-[[4-chloro-3- (trifluoromethyl)phenyl]carbamoyl- amino] phenoxy]-N-methyl-pyridine-2- carboxamide | 284461- 73-0 | 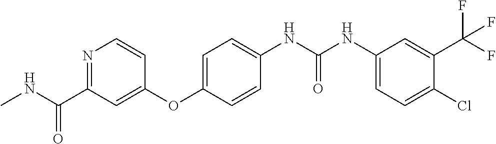 [SMILES: CNC(=O)c1cc(Oc2ccc(NC(=O)Nc3ccc(Cl)c(C(F)(F)F)c3)cc2)ccn1] |
| 4 | SB590885 | 405554- 55-4 | 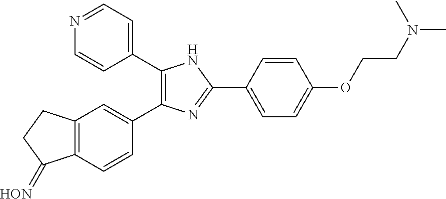 |
| 5 | PLX4720 | 918505- 84-7 | 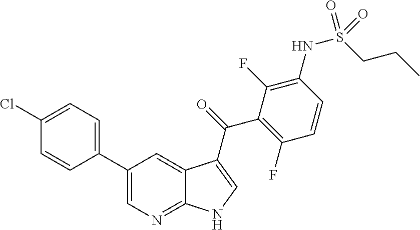 [SMILES: CCCS(=O)(=O)Nc1ccc(F)c(C(=O)c2c[nH]c3ncc(-c4ccc(Cl)cc4)cc23)c1F] |
| 6 | PLX4032 | 1029872- 54-5 | 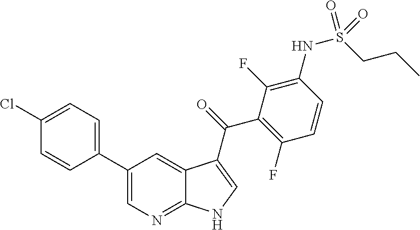 [SMILES: CCCS(=O)(=O)Nc1ccc(F)c(C(=O)c2c[nH]c3ncc(-c4ccc(Cl)cc4)cc23)c1F] |
| 7 | GDC-0879 | 905281- 76-7 | 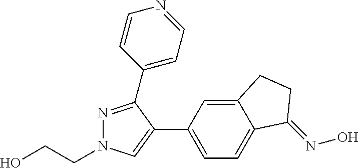 [SMILES: OCCn1cc(-c2ccc3c(c2)CC/C3=N\O)c(-c2ccncc2)n1] |

Examples of RAF inhibitors therefore include PLX4720, PLX4032, BAY 43-9006 (Sorafenib), ZM 336372, RAF 265, AAL-881, LBT-613, or CJS352 (NVP-AAL881-NX (hereafter referred to as AAL881) and NVP-LBT613-AG-8 (LBT613) are isoquinoline compounds (Novartis, Cambridge, Mass.). Additional exemplary RAF inhibitors useful for combination therapy include pan-RAF inhibitors, inhibitors of B-RAF, inhibitors of A-RAF, and inhibitors of RAF-1. In exemplary embodiments RAF inhibitors useful for combination therapy include PLX4720, PLX4032, BAY 43-9006 (Sorafenib), ZM 336372, RAF 265, AAL-881, LBT-613, and CJS352. Exemplary RAF inhibitors further include the compounds set forth in PCT Publication No. WO/2008/028141 and WO2011/027689, the specific teachings of which are incorporated herein by reference. Exemplary RAF inhibitors additionally include the quinazolinone derivatives described in PCT Publication No. WO/2006/024836, and the pyridinylquinazolinamine derivatives described in PCT Publication No. WO/2008/020203, the specific inhibitor teachings of which are incorporated herein by reference.

TABLE 7
Exemplary MEK Inhibitors

| | Name | CAS No. | Structure |
|---|---|---|---|
| 1 | CI-1040/PD184352 | 212631- 79-3 | 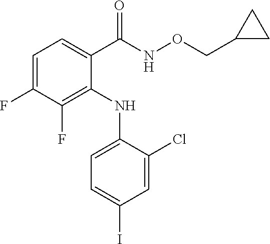 [SMILES: O=C(NOCC1CC1)c1ccc(F)c(F)c1Nc1ccc(I)cc1Cl] |
| 2 | AZD6244 | 606143- 52-6 | 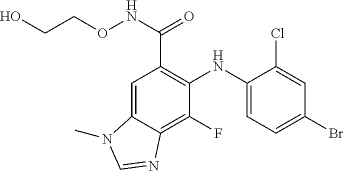 [SMILES: Cn1cnc2c(F)c(Nc3ccc(Br)cc3Cl)c(C(=O)NOCCO)cc21] |
| 3 | PD318088 | 391210- 00-7 | 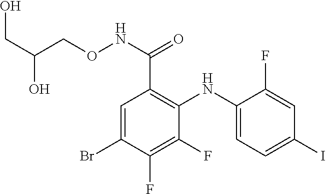 [SMILES: O=C(NOCC(O)CO)c1cc(Br)c(F)c(F)c1Nc1ccc(I)cc1F] |
| 4 | PD98059 | 167869- 21-8 | 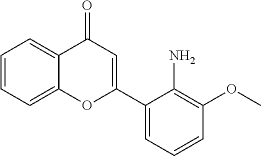 [SMILES: COc1cccc(-c2cc(=O)c3ccccc3o2)c1N] |
| 5 | PD334581 | | 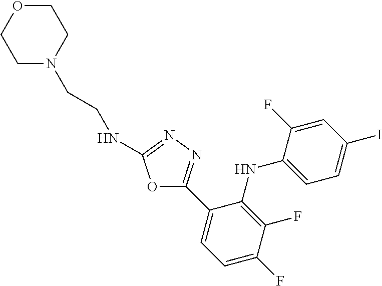 [SMILES: Fc1cc(I)ccc1Nc1c(-c2nnc(NCCN3CCOCC3)o2)ccc(F)c1F] |
| 6 | RDEA119 N-[3,4-difluoro-2-[(2- fluoro-4- iodophenyl)amino]-6- methoxyphenyl]-1-[(2R)- 2,3-dihydroxypropyl]- Cyclopropanesulfonamide | 923032- 38-6 | 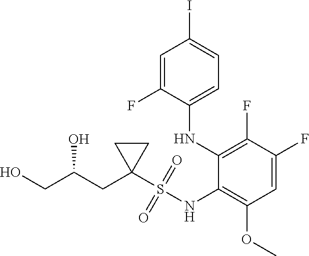 [SMILES: COc1cc(F)c(F)c(Nc2ccc(I)cc2F)c1NS(=O)(=O)C1(C[C@@H](O)CO)CC1] |

Additional MEK inhibitors include the compounds described in the following patent publications, the specific inhibitor teachings of which are incorporated herein by reference: WO 2008076415, US 20080166359, WO 2008067481, WO 2008055236, US 20080188453, US 20080058340, WO 2007014011, WO 2008024724, US 20080081821, WO 2008024725, US 20080085886, WO 2008021389, WO 2007123939, US 20070287709, WO 2007123936, US 20070287737, US 20070244164, WO 2007121481, US 20070238710, WO 2007121269, WO 2007096259, US 20070197617, WO 2007071951, EP 1966155, IN 2008MN01163, WO 2007044084, AU 2006299902, CA 2608201, EP 1922307, EP 1967516, MX 200714540, IN 2007DN09015, NO 2007006412, KR 2008019236, WO 2007044515, AU 2006302415, CA 2622755, EP 1934174, IN 2008DN02771, KR 2008050601, WO 2007025090, US 20070049591, WO 2007014011, AU 2006272837, CA 2618218, EP 1912636, US 20080058340, MX 200802114, KR 2008068637, US 20060194802, WO 2006133417, WO 2006058752, AU 2005311451, CA 2586796, EP 1828184, JP 2008521858, US 20070299103, NO 2007003393, WO 2006056427, AU 2005308956, CA 2587178, EP 1838675, JP 2008520615, NO 2007003259, US 20070293544, WO 2006045514, AU 2005298932, CA 2582247, EP 1802579, CN 101065358, JP 2008517024, IN 2007DN02762, MX 200704781, KR 2007067727, NO 2007002595, JP 2006083133, WO 2006029862, US 20060063814, U.S. Pat. No. 7,371,869, AU 2005284293, CA 2579130, EP 1791837, CN 101023079, JP 2008513397, BR 2005015371, KR 2007043895, MX 200703166, IN 2007CN01145, WO 2006024034, AU 2005276974, CA 2578283, US 20060079526, EP 1799656, CN 101044125, JP 2008510839, MX 200702208, IN 2007DN02041, WO 2006018188, AU 2005274390, CA 2576599, EP 1781649, CN 101006085, JP 2008509950, BR 2005014515, AT 404556, US 20060041146, MX 200701846, IN 2007CN00695, KR 2007034635, WO 2006011466, AU 2005265769, CA 2575232, EP 1780197, BR 2005013750, JP 4090070, MX 200700736, CN 101124199, KR 2007041752, IN 2007DN01319, WO 2005121142, AU 2005252110, CA 2569850, US 20060014768, U.S. Pat. No. 7,378,423, EP 1761528, CN 101006086, AT 383360, BR 2005011967, JP 2008501631, EP 1894932, ES 2297723, MX 2006PA14478, NO 2007000155, IN 2007CN00102, KR 2007034581, HK 1107084, JP 2008201788, US 20050256123, US 20050250782, US 20070112038, US 20050187247, WO 2005082891, WO 2005051302, AU 2004293019, CA 2546353, US 20050130943, US 20050130976, US 20050153942, EP 1689233, JP 2007511615, WO 2005051301 AU 2004293018, CA 2545660, US 20050130943, US 20050130976, US 20050153942, EP 1682138, BR 2004016692, CN 1905873, JP 2007511614, MX 2006PA05657, IN 2006DN03183, NO 2006002692, KR 2007026343, WO 2005028426, EP 1674452, US 20070105859, US 20050054701, U.S. Pat. No. 7,230,099, US 20050049419, U.S. Pat. No. 7,144,907, AU 2004270699, CA 2537321, WO 2005023759, EP 1673339, BR 2004014111, CN 1874769, JP 2007504241, JP 4131741, US 20060030610, MX 2006PA02466, NO 2006001506, US 20050049419, U.S. Pat. No. 7,144,907, US 20050054701, U.S. Pat. No. 7,230,099, AU 2004270699, CA 2537321, WO 2005023759, EP 1673339, BR 2004014111, CN 1874769, JP 2007504241, JP 4131741 US 20060030610, MX 2006PA02466, IN 2006DN01661, NO 2006001506, US 20060189808, US 20060189649, U.S. Pat. No. 7,271,178, US 20060189668, US 20050049276, WO 2005009975, CA 2532067, EP 1651214, BR 2004012851, JP 2006528621, US 20050026970, U.S. Pat. No. 7,160,915, MX 2006PA00921, WO 2005007616, US 20050059710, WO 2005000818, US 20050026964, U.S. Pat. No. 7,273,877, WO 2004056789, US 20050004186, CA 2509405, AU 2003286306, EP 1578736, BR 2003017254, JP 2006516967, MX 2005PA06803, WO 2004044219, AU 2003291268, WO 2004041811, AU 2003278369, EP 1575943, JP 2006508944, US 20050282856, U.S. Pat. No. 7,173,136, US 20070191346, WO 2004041185, AU 2003287366, US 20060270643, WO 2004030620, AU 2003275282, US 20040092514, U.S. Pat. No. 7,232,826, EP 1545529, US 20040039037, U.S. Pat. No. 6,989,451, WO 2003077855, CA 2478534, AU 2003220202, US 20030216460, EP 1482944, CN 1652792, JP 2005526076, RU 2300528, BR 2003006016, MX 2004PA08894, US 20060106225, WO 2003062191, CA 2473545, EP 1467968, BR 2003007060, JP 2005515253, TW 592692, US 20040006245, U.S. Pat. No. 6,891,066, MX 2004PA05527, US 20050137263, U.S. Pat. No. 7,078,438, WO 2003062189, CA 2472367, EP 1467965, BR 2003007071, JP 2005515251, US 20030232889, U.S. Pat. No. 6,770,778, MX 2004PA05528, WO 2003047585, AU 2002347360, WO 2003047583, AU 2002365665, WO 2003047523, CA 2466762, AU 2002365899, US 20030125359, U.S. Pat. No. 7,307,071, JP 2005526008, EP 1578346, WO 2003035626, CA 2463101, AU 2002359291, EP 1438295, JP 2005508972, US 20050054706, U.S. Pat. No. 7,253,199, US 20070293555, AU 2008202731, U.S. Pat. No. 6,506,798, WO 9901421, WO 2000041994, U.S. Pat. No. 6,310,060, WO 2002006213, CA 2416685, AU 2001073498, BR 2001012584, HU 2003002781, JP 2004504294, JP 3811775, EE 200300030, NZ 524120, AU 2001273498, IN 2003MN00028, NO 2003000249, KR 773621, MX 2003PA00591, HR 2003000083, BG 107564, ZA 2003000348, US 20040054172, U.S. Pat. No. 6,960,614, HK 1055943, US 20050176820, U.S. Pat. No. 7,411,001, WO 2000042029, JP 2000204077, CA 2355374, EP 1144394, BR 9916896, TR 200102029, HU 2001005092, JP 2002534515, EE 200100374, NZ 513432, AT 302761, ES 2249060, ZA 2001005219, MX 2001 PA06659, IN 2001MN00785, NO 2001003451, HR 2001000525, BG 105801, U.S. Pat. No. 6,545,030, HK 1042488, US 20030004193, WO 2000042022, JP 2000204079, CA 2355470, EP 1144385, BR 9916904, TR 200102030, HU 2001005113, EE 200100373, JP 2002534510, NZ 513433, AT 302193, ES 2247859, MX 2001 PA06568, ZA 2001005224, IN 2001MN00786, U.S. Pat. No. 6,469,004, NO 2001003452, HR 2001000524, BG 105800, WO 2000042003, JP 2000212157, CA 2349832, EP 1144371, BR 9916885, AT 309205, ES 2252996, MX 2001 PA04332, U.S. Pat. No. 6,440,966, US 20030092748, U.S. Pat. No. 6,750,217, WO 2000042002, JP 2000204075, CA 2349467, EP 1144372, BR 9916894, JP 2002534497, AT 311363, ES 2251851, MX 2001 PA04331, U.S. Pat. No. 6,455,582, US 20030045521, U.S. Pat. No. 6,835,749, WO 2000037141, CA 2352326, BR 9916839, EP 1140291, TR 200101871, HU 2001004844, JP 2002532570, EE 200100339, NZ 512859, AT 310567, ES 2253928, ZA 2001004277, MX 2001 PA05476, IN 2001MN00673, NO 2001003099, HR 2001000473, BG 105715, US 20040171632, GB 2323845, WO 9901426, CA 2290506, AU 9882627, AU 757046, EP 993439, BR 9810366, NZ 501276, HU 2000003731, JP 2002511092, AT 277895, IL 132840, PT 993439, ES 2229515, TW 396149, ZA 9805728, MX 9910649, NO 9906491, NO 315271, US 20030078428, U.S. Pat. No. 6,821,963, US 20050049429, US 20060052608, U.S. Pat. No. 7,169,816, WO 9901421, CA 2290509, AU 9882626, AU 756586, EP 993437, BR 9810385, JP 2002509536, NZ 501277, AT 344791, ES 2274572, TW 221831, ZA 9805726, MX 9910556, U.S. Pat. No. 6,310,060, U.S. Pat. No. 6,506,798, US 20020022647, U.S. Pat. No. 6,492,363, US 20030149015, and U.S. Pat. No. 7,019,033.

TABLE 8
Exemplary ERK Inhibitors

| | Name | CAS No. | Structure |
|---|---|---|---|
| 1 | VTX11e | | 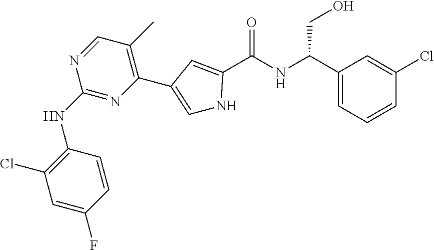 [SMILES: Cc1cnc(Nc2ccc(F)cc2Cl)nc1-c1c[nH]c(C(=O)N[C@H](CO)c2cccc(Cl)c2)c1] |
| 2 | PD98059 | 167869- 21-8 | 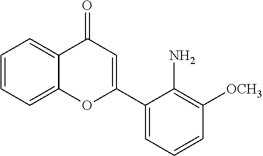 [SMILES: COc1cccc(-c2cc(=O)c3ccccc3o2)c1N] |
| 3 | FR180204 | 865362- 74-9 | 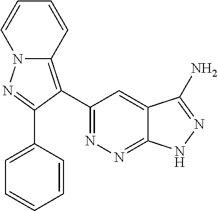 [SMILES: Nc1n[nH]c2nnc(-c3c(-c4ccccc4)nn4ccccc34)cc12] |
| 4 | FR148083 (5Z-7-oxozeaenol) | 253863- 19-3 | 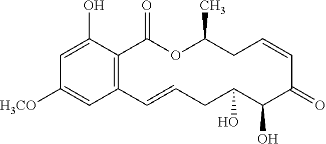 [SMILES: COc1cc(O)c2c(c1)/C=C/C[C@H](O)[C@H](O)C(=O)/C=C\C[C@H](C)OC2=O] |

Additional ERK inhibitors include the compounds described in the following patents and patent publications, the specific inhibitor teachings of which are incorporated herein by reference: US 20120214823, US20070191604, US20090118284, US20110189192, U.S. Pat. No. 6,528,509, EP2155722A1, and EP2170893A1.

TABLE 9
Exemplary HDAC Inhibitors

| | Name | CAS No. | Structure |
|---|---|---|---|
| 1 | Vorinostat | 149647- 78-9 | 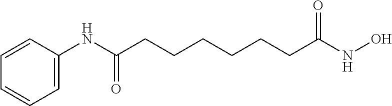 [SMILES: O=C(CCCCCCC(=O)Nc1ccccc1)NO] |
| 2 | CI-994 | 112522- 64-2 | 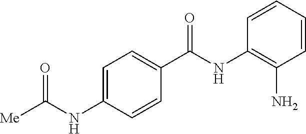 [SMILES: CC(=O)Nc1ccc(C(=O)Nc2ccccc2N)cc1] |
| 3 | Entinostat | 209783- 80-2 | 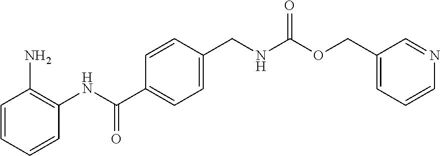 [SMILES: Nc1ccccc1NC(=O)c1ccc(CNC(=O)OCc2cccnc2)cc1] |
| 4 | BML-210 | 537034- 17-6 | 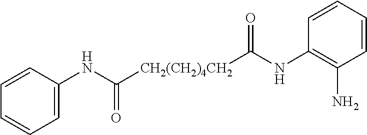 |
| 5 | M344 | 251456- 60-7 | 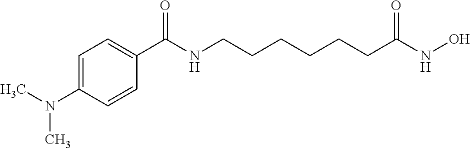 [SMILES: CN(C)c1ccc(C(=O)NCCCCCCC(=O)NO)cc1] |
| 6 | NVP-LAQ824 | 404951- 53-7 | 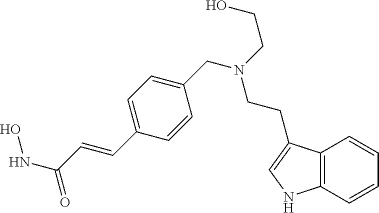 [SMILES: O=C(/C=C/c1ccc(CN(CCO)CCc2c[nH]c3ccccc23)cc1)NO] |
| 7 | Panobinostat | 404950- 80-7 | 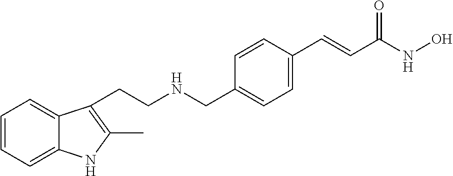 [SMILES: Cc1[nH]c2ccccc2c1CCNCc1ccc(/C=C/C(=O)NO)cc1] |
| 8 | Mocetinostat | 726169- 73-9 | 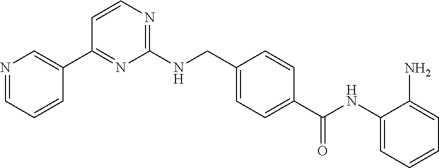 [SMILES: Nc1ccccc1NC(=O)c1ccc(CNc2nccc(-c3cccnc3)n2)cc1] |

Additional HDAC inhibitors include the compounds described in the following patents and patent publications, the specific inhibitor teachings of which are incorporated herein by reference: EP2456757A2, US20120252740, EP2079462A2, EP2440517A2, U.S. Pat. No. 8,258,316, EP2049505A2, US20130040998,U.S. Pat. No. 8,283,357, EP2292593A3, EP1888097A1, EP2330894A1, EP1745022A1, EP2205563A2, U.S. Pat. No. 8,143,445, US20130018103, EP1758847A1, U.S. Pat. No. 7,135,493, EP1789381A2, EP1945617A2, U.S. Pat. No. 7,557,127, U.S. Pat. No. 8,293,513, US20100196502, US20070088043, US20120208889, EP1943232A1, US20070129290, U.S. Pat. No. 7,569,724, EP1524262A1, EP1280764B1, EP1495002B1, EP1485364A1, U.S. Pat. No. 7,557,140, U.S. Pat. No. 7,407,988, U.S. Pat. No. 8,338,416, US20120178783, U.S. Pat. No. 7,183,298, EP1881977B1, US20100261710, US20090054448, US20050118596, EP2265590A2, U.S. Pat. No. 8,188,054, US20110105474, US20110237832, US20100010010, U.S. Pat. No. 7,423,060, EP2197854A1, U.S. Pat. No. 7,973,181, EP1773398A2, US20120329741, US20120094971, EP2069291 A1, EP2436382A1, US20090136431, and US20110105572.

### Diagnostic/Prognostic/Theranostic Methods

The invention therefore provides methods of detecting the presence of one or more predictive, diagnostic or prognostic markers in a sample (e.g., a biological sample from a cancer patient). A variety of screening methods known to one of skill in the art may be used to detect the presence and the level of the marker in the sample including DNA, RNA and protein detection. The techniques described herein can be used to determine the presence or absence of a target in a sample obtained from a patient.

In some embodiments, the patient may have innate or acquired resistance to kinase targeted therapies, including RAF inhibitors, MEK inhibitors, and/or ERK inhibitors. For example, the patient may have an innate or acquired resistance to B-RAF inhibitors PLX4720 and/or PLX4032. In some embodiments, the patient may have innate or acquired resistance to MEK inhibitor AZD6244. In some embodiments, the patient may have innate or acquired resistance to ERK inhibitor VTX11e.

As used herein, “resistance” includes a non-responsiveness or decreased responsiveness in a subject to treatment with an inhibitor. Non-responsiveness or decreased responsiveness may include an absence or a decrease of the benefits of treatment, such as a decrease or cessation of the relief, reduction or alleviation of at least one symptom of the disease in the subject. For example, in a subject having a cancer that in not resistant to (i.e. sensitive to) a MAPK pathway inhibitor, administration of the inhibitor to the subject may result in a reduction of tumor burden or complete eradication of the cancer. On the other hand, in a subject having a cancer resistant to a MAPK pathway inhibitor, administration of the inhibitor to the subject may result in a smaller or no reduction of tumor burden or no eradication of the cancer.

As used herein, “innate resistance” includes a subject having a cancer that is naturally resistant to an inhibitor. As used herein, “acquired resistance” includes a subject having a cancer that develops resistance to an inhibitor after administration of the inhibitor to the subject.

Identification of one or more markers (including identification of elevated levels of one or more markers) in a patient assists a physician or other medical professional in determining a treatment protocol for the patient. For example, in a patient having one or more markers, the physician may treat the patient with a combination therapy as described in more detail below. Alternatively, the physician may choose to administer a different therapy altogether to the patient.

In some embodiments, the marker is selected from a GPCR that activates production of cAMP and a GPCR pathway component selected from FOS, NR4A1, NR4A2, and MITF, and a PKA-activated transcription factor that activates FOS, NR4A1, NR4A2, and MITF. The marker may be evaluated for an increase in gene copy number, an increase in mRNA expression, an increase in protein expression, and/or an increase in activity.

In some embodiments, the marker is a GEF. The GEF may be ARHGEF2, ARHGEF3, ARHGEF9, ARHGEF19, IQSEC1, MCF2L, NGEF, PLEKHG3, PLEKHG5, PLEKHG6, TBC1 D3G, SPATA13, or VAV1, or it may be any of the GEFs recited herein or known in the art. The marker may be evaluated for an increase in gene copy number, an increase in mRNA expression, an increase in protein expression, and/or an increase in activity such as but not limited to an increase in the level of one or more active GTPases.

By way of non-limiting example, in a patient having an oncogenic mutation in B-RAF, identification of a resistance-conferring marker can be useful for determining a treatment protocol for the patient. For example, in a patient having a B-RAF^{V600E} mutation, treatment with a RAF inhibitor alone, an ERK inhibitor alone, or a combination of a RAF and ERK inhibitor may indicate that the patient is at relatively high risk of acquiring resistance to the treatment after a period of time. In a patient having an oncogenic mutation, identification of an increased level and/or activity of one or more markers in that patient may indicate inclusion of a second inhibitor such as a GEF inhibitor or an HDAC inhibitor in the treatment protocol.

Identification of an increased level and/or activity of one or more markers selected from a GPCR that activates production of cAMP, a GPCR pathway component selected from FOS, NR4A1, NR4A2, and MITF, and a PKA-activated transcription factor that activates FOS, NR4A1, NR4A2, and MITF may include an analysis of a gene copy number and identification of an increase in copy number of the one or more markers.

Identification of an increased level and/or activity of one or more markers selected from a GPCR that activates production of cAMP, a GPCR pathway component selected from FOS, NR4A1, NR4A2, and MITF, and a PKA-activated transcription factor that activates FOS, NR4A1, NR4A2 may include an analysis of mRNA expression or protein expression of the one or more markers. For example, an increase in mRNA expression of the one or more markers is indicative of (a) a patient at risk of developing resistance to a MAPK pathway inhibitor and who optionally may be treated with an HDAC inhibitor alone or in combination with another therapy such as a RAF inhibitor, a MEK inhibitor, and/or an ERK inhibitor or (b) a patient who is resistant to a MAPK pathway inhibitor and who should be treated with an HDAC inhibitor alone or in combination with another therapy such as a RAF inhibitor, a MEK inhibitor, and/or an ERK inhibitor.

Identification of an increased level and/or activity of one or more GEFs may include an analysis of a gene copy number and identification of an increase in copy number of one or more GEFs. For example, a copy number gain in one or more GEFs (e.g., VAV1) is indicative of a patient having innate resistance or at risk of developing acquired resistance to a MAPK pathway inhibitor such as a RAF inhibitor or a MEK inhibitor. This is particularly the case if the patient also has a B-RAF^{V600E} mutation.

Identification of an increased level and/or activity of one or more GEFs may include an analysis of one or more GTPases, including the active status of one or more GTPases. In some instances, an increase in the level of active GTPases (i.e., GTPase-GTP) is indicative of a patient having innate resistance or at risk of developing acquired resistance, particularly if the patient also has a B-RAF^{V600E} mutation.

Identification of an increased level and/or activity of one or more GEFs may include an analysis of mRNA expression or protein expression of one or more GEFs. For example, an increase in mRNA expression of one or more GEFs (e.g., VAV1) is indicative of (a) a patient at risk of developing resistance to a MAPK pathway inhibitor and who optionally may be treated with a GEF inhibitor alone or in combination with another therapy such as a RAF inhibitor and/or a MEK inhibitor, or (b) a patient who is resistant to a MAPK pathway inhibitor and who should be treated with a GEF inhibitor alone or in combination with another therapy such as a RAF inhibitor and/or a MEK inhibitor.

### Treatment Methods

The term “treat”, “treated,” “treating” or “treatment” is used herein to mean to relieve, reduce or alleviate at least one symptom of a disease in a subject. For example, treatment can be diminishment of one or several symptoms of a disorder or complete eradication of a disorder, such as cancer. Within the meaning of the present invention, the term “treat” also denote to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. Within the meaning of the present invention, the disease is associated with a cancer.

The term “subject” or “patient” is intended to include animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats, and transgenic non-human animals. In certain embodiments, the subject is a human, e.g., a human having, at risk of having, or potentially capable of having cancer.

The term “cancer” is used herein to mean malignant solid tumors as well as hematological malignancies. In some instances, the cancer is melanoma. The melanoma may be metastatic melanoma. Additional examples of such tumors include but are not limited to leukemias, lymphomas, myelomas, carcinomas, metastatic carcinomas, sarcomas, adenomas, nervous system cancers and genitourinary cancers. In exemplary embodiments, the foregoing methods are useful in treating adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, osteosarcoma, fibrous histiocytoma, brain cancer, brain stem glioma, cerebellar astrocytoma, malignant glioma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, and Wilms tumor.

In particular, the cancer may be associated with a mutation in the B-RAF gene. These cancers include melanoma, breast cancer, colorectal cancers, glioma, lung cancer, ovarian cancer, sarcoma and thyroid cancer.

The invention provides methods of treatment of a patient having cancer. Typically, the patient is identified as one who has increased marker level or activity, such as a GEF level or activity or a level or activity of a marker selected from a GPCR that activates production of cAMP, a GPCR pathway component selected from FOS, NR4A1, NR4A2, and MITF, and a PKA-activated transcription factor that activates FOS, NR4A1, NR4A2. The methods may comprise administration of one or more GEF inhibitors or HDAC inhibitors in the absence of a second therapy.

Other methods of the invention comprise administration of a first inhibitor and a second inhibitor. The designation of “first” and “second” inhibitors is used to distinguish between the two and is not intended to refer to a temporal order of administration of the inhibitors.

The first inhibitor may be a RAF inhibitor. The RAF inhibitor may be a pan-RAF inhibitor or a selective RAF inhibitor. Pan-RAF inhibitors include but are not limited to RAF265, sorafenib, and SB590885. In some embodiments, the RAF inhibitor is a B-RAF inhibitor. In some embodiments, the selective RAF inhibitor is PLX4720, PLX4032, Dabrafenib, or GDC-0879-A. Other RAF inhibitors are provided herein.

The first inhibitor may be a MEK inhibitor. MEK inhibitors include but are not limited to CI-1040, AZD6244, PD318088, PD98059, PD334581, RDEA119, 6-Methoxy-7-(3-morpholin-4-yl-propoxy)-4-(4-phenoxy-phenylamino)-quinoline-3-carbonitrile or 4-[3-Chloro-4-(1-methyl-1H-imidazol-2-ylsulfanyl)-phenylamino]-6-methoxy-7-(3-morpholin-4-yl-propoxy)-quinoline-3-carbonitrile, Roche compound RG7420, Trametinib, or combinations thereof. In some embodiments, the MEK inhibitor is CI-1040/PD184352 or AZD6244. Other MEK inhibitors are provided herein.

The first inhibitor may be an ERK inhibitor. ERK inhibitors include but are not limited to VTX11e, AEZS-131, PD98059, FR180204, FR148083, or combinations thereof. In some embodiments, the ERK inhibitor is VTX11e. Other ERK inhibitors are provided herein.

It is to be understood that a combination of MAPK pathway inhibitors may be used such as a combination of a RAF inhibitor and a MEK inhibitor. In some embodiments, the RAF inhibitor is Dabrafenib and the MEK inhibitor is Trametinib.

The second inhibitor may be an HDAC inhibitor. HDAC inhibitors include but are not limited Vorinostat, CI-994, Entinostat, BML-210, M344, NVP-LAQ824, Panobinostat, Mocetinostat, Belinostat, or combinations thereof. In some embodiments, the HDAC inhibitor is Panobinostat, Vorinostat, or Entinostat. Other HDAC inhibitors are provided herein.

Thus, in some embodiments, a combination therapy for cancer is provided, comprising an effective amount of a RAF inhibitor and an HDAC inhibitor. The RAF inhibitor may be a pan-RAF inhibitor or it may be a selective RAF inhibitor.

In other embodiments, a combination therapy for cancer is provided comprising an effective amount of a RAF inhibitor, a MEK inhibitor, and an HDAC inhibitor. The RAF inhibitor may be a pan-RAF inhibitor or it may be a selective RAF inhibitor.

In other embodiments, a combination therapy for cancer is provided comprising an effective amount of (i) a RAF inhibitor, a MEK inhibitor, and/or an ERK inhibitor and (ii) an HDAC inhibitor. The RAF inhibitor may be a pan-RAF inhibitor or it may be a selective RAF inhibitor.

The second inhibitor may be a GEF inhibitor. The GEF inhibitor may target the GEF gene, GEF mRNA expression, GEF protein expression, and/or GEF activity, in all instances reducing the level and/or activity of one or more GEFs. GEF inhibitors may be nucleic acids such as DNA and RNA aptamers, antisense oligonucleotides, siRNA and shRNA, small peptides, antibodies or antibody fragments, and small molecules such as small chemical compounds. GEF inhibitors are known in the art. Examples of aptamers are provided in published US patent application number US 20090036379, granted U.S. Pat. No. 8,088,892, published EP patent application numbers EP 1367064 and EP 1507797 (describing, inter alia, Rho-GEF inhibitors). Examples of antibodies and antibody fragments specific for GEF and useful as inhibitors of GEFs are described in granted U.S. Pat. No. 7,994,294 (describing, inter alia, antibodies to Rho-GEF). Other specific examples of GEF inhibitors include but are not limited to ITX-3 (a selective cell active inhibitor or TRIO/RhoG/Rac1 pathway), TRIO-GEFD1, Brefeldin (a natural GEF inhibitor), TRIPalpha (an inhibitor of Rho-GEF), and 3-(3-(dihydroxy(oxido)stibino)phenyl)acrylic acid (NSC#13778; Stibinophenyl acrylic acid). Other examples of GEF inhibitors include the VAV inhibitors described in published PCT application number WO2004/091654, the Asef inhibitors described in granted U.S. Pat. No. 7,297,779. The specific inhibitor teachings of each of these references is incorporated by reference herein.

GEF inhibitors of the invention may inhibit one or more GEF targets such as but not limited to ARHGEF2, ARHGEF3, ARHGEF9, ARHGEF19, IQSEC1, MCF2L, NGEF, PLEKHG3, PLEKHG5, PLEKHG6, TBC1 D3G, SPATA13, and VAV1.

In other embodiments, the second inhibitor may be an inhibitor of a GTPase, or an inhibitor of a kinase downstream of the GTPase such as but not limited to a PAK, a Rho kinase, and a Rhotekin. The GTPase inhibitor may target the GTPase gene, GTPase mRNA expression, GTPase protein expression, and/or GTPase activity. The kinase inhibitor may target the kinase gene, kinase mRNA expression, kinase protein expression, and/or kinase activity.

Thus, in some embodiments, a combination therapy for cancer is provided, comprising an effective amount of a RAF inhibitor and a GEF inhibitor. The RAF inhibitor may be a pan-RAF inhibitor or it may be a selective RAF inhibitor.

In other embodiments, a combination therapy for cancer is provided comprising an effective amount of a RAF inhibitor, a MEK inhibitor, and a GEF inhibitor. The RAF inhibitor may be a pan-RAF inhibitor or it may be a selective RAF inhibitor.

In other embodiments, a combination therapy for cancer is provided comprising an effective amount of (i) a RAF inhibitor, a MEK inhibitor, and/or an ERK inhibitor and (ii) a GEF inhibitor. The RAF inhibitor may be a pan-RAF inhibitor or it may be a selective RAF inhibitor.

Any of the therapies including combination therapies described herein are suitable for the treatment of a patient manifesting resistance to a MAPK pathway inhibitor such as a RAF inhibitor or a MEK inhibitor or a patient likely to manifest resistance to such inhibitors. The patient may have a cancer characterized by the presence of a B-RAF mutation. The B-RAF mutation may be but is not limited to B-RAF^{V600E}. The cancer may be but is not limited to melanoma.

### Pharmaceutical Formulations, Administration and Dosages

Provided herein are pharmaceutical formulations comprising single agents, such as HDAC or GEF inhibitors (and/or pharmacologically active metabolites, salts, solvates and racemates thereof).

In other instances, provided herein are pharmaceutical formulations comprising a combination of agents which can be, for example, a combination of two types of agents such as a RAF inhibitor and/or pharmacologically active metabolites, salts, solvates and racemates thereof in combination with (1) an HDAC inhibitor and/or pharmacologically active metabolites, salts, solvates and racemates thereof, or (2) a GEF inhibitor and/or pharmacologically active metabolites, salts, solvates and racemates thereof.

In another embodiment, the combination may be of three types of agents: (1) a RAF inhibitor and/or pharmacologically active metabolites, salts, solvates and racemates thereof, (2) a MEK inhibitor and/or pharmacologically active metabolites, salts, solvates and racemates thereof, and (3) an HDAC inhibitor and/or pharmacologically active metabolites, salts, solvates and racemates thereof. Another suitable combination comprises (1) a RAF inhibitor and/or pharmacologically active metabolites, salts, solvates and racemates thereof, (2) a MEK inhibitor and/or pharmacologically active metabolites, salts, solvates and racemates thereof, and (3) a GEF inhibitor and/or pharmacologically active metabolites, salts, solvates and racemates thereof.

Agents may contain one or more asymmetric elements such as stereogenic centers or stereogenic axes, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms; all isomeric forms of the compounds are included in the present invention. In these situations, the single enantiomers (optically active forms) can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Unless otherwise specified, or clearly indicated by the text, reference to compounds useful in the therapeutic methods of the invention includes both the free base of the compounds, and all pharmaceutically acceptable salts of the compounds. The term “pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, and nitric; other acceptable inorganic salts include metal salts such as sodium salt, potassium salt, and cesium salt; and alkaline earth metal salts, such as calcium salt and magnesium salt; and combinations comprising one or more of the foregoing salts. In some embodiments, the salt is a hydrochloride salt.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC(CH₂)ₙCOOH where n is 0-4; organic amine salts such as triethylamine salt, pyridine salt, picoline salt, ethanolamine salt, triethanolamine salt, dicyclohexylamine salt, N,N′-dibenzylethylenediamine salt; and amino acid salts such as arginate, asparginate, and glutamate, and combinations comprising one or more of the foregoing salts.

The agents of the invention are administered in effective amounts. An “effective amount” is an amount sufficient to provide an observable improvement over the baseline clinically observable signs and symptoms of the disorder treated with the combination. An effective amount of an inhibitor such as a GEF inhibitor may be determined in the presence or absence of one or more other inhibitors such as RAF inhibitors and/or MEK inhibitors.

The effective amount may be determined using known methods and will depend upon a variety of factors, including the activity of the agents; the age, body weight, general health, gender and diet of the subject; the time and route of administration; and other medications the subject is taking. Effective amounts may be established using routine testing and procedures that are well known in the art.

A physician or veterinarian having ordinary skill in the art can readily determine and prescribe the effective amount of the pharmaceutical composition required. For example, the physician or veterinarian could start at doses lower than those required to achieve the desired therapeutic effect and gradually increase the dosage until the desired effect is achieved. In general, a suitable daily dose of will be that amount of the compound that is the lowest dose effective to produce a therapeutic effect.

Generally, therapeutically effective doses of the compounds of this invention for a patient will range from about 0.0001 to about 1000 mg per kilogram of body weight per day, more preferably from about 0.01 to about 50 mg per kg per day.

If desired, the effective daily dose of the active compound may be administered as two, three, four, five, six or more sub-doses administered separately at appropriate intervals throughout the day, optionally, in unit dosage forms.

The agents may be administered using a variety of routes of administration known to those skilled in the art. The agents may be administered to humans and other animals orally, parenterally, sublingually, by aerosolization or inhalation spray, rectally, intracisternally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agents as compared to the other agent in the combination. Therefore, to permit appropriate dosing, packaged pharmaceutical products may contain one or more dosage forms that contain the combination of agents, and one or more dosage forms that contain one of the combinations of agents, but not the other agent(s) of the combination. Administration may be concurrent or sequential.

The pharmaceutical formulations may additionally comprise a carrier or excipient, stabilizer, flavoring agent, and/or coloring agent. Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono or di glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

The pharmaceutical products can be released in various forms. “Releasable form” is meant to include instant release, immediate-release, controlled-release, and sustained-release forms.

“Instant-release” is meant to include a dosage form designed to ensure rapid dissolution of the active agent by modifying the normal crystal form of the active agent to obtain a more rapid dissolution.

“Immediate-release” is meant to include a conventional or non-modified release form in which greater than or equal to about 50% or more preferably about 75% of the active agents is released within two hours of administration, preferably within one hour of administration.

“Sustained-release” or “extended-release” includes the release of active agents at such a rate that blood (e.g., plasma) levels are maintained within a therapeutic range but below toxic levels for at least about 8 hours, preferably at least about 12 hours, more preferably about 24 hours after administration at steady-state. The term “steady-state” means that a plasma level for a given active agent or combination of active agents, has been achieved and which is maintained with subsequent doses of the active agent(s) at a level which is at or above the minimum effective therapeutic level and is below the minimum toxic plasma level for a given active agent(s).

The pharmaceutical products can be administrated by oral dosage form. “Oral dosage form” is meant to include a unit dosage form prescribed or intended for oral administration. An oral dosage form may or may not comprise a plurality of subunits such as, for example, microcapsules or microtablets, packaged for administration in a single dose.

Compositions for rectal or vaginal administration are preferably suppositories which can be prepared by mixing the compounds of this invention with suitable non irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 microns. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1 to 5 microns. Predominantly in this application means that at least 70% but preferably more than 90% of all generated aerosol particles are within 1 to 5 micron range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono or multi lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

### Devices

Other aspects of the invention relate to devices. In some embodiments, the device comprises a sample inlet and a substrate, wherein the substrate comprises one or more binding partners for one or more markers as described herein. In some embodiments, the device is a microarray.

It is to be understood that the device may comprise binding partners for any combination of markers described herein or that can be contemplated by one of ordinary skill in the art based on the teachings provided herein.

The device may also comprise binding partners for one or more control markers. The control markers may be positive control markers (e.g., to ensure the device has maintained its integrity) and/or negative control markers (e.g., to identify contamination or to ensure the device has maintained its specificity). The nature of the control markers will depend in part on the nature of the biological sample.

The device may comprise binding partners for 1-150, 1-100, 1-50, 1-20, 1-10, 1-5, 2-150, 2-100, 2-50, 2-20, 2-10, 2-5, 3-150, 3-100, 3-50, 3-20, 3-10, 3-5, 4-150, 4-100, 4-50, 4-20, 4-10, 5-150, 5-100, 5-50, 5-20, 1-150, 1-100, 1-50, 1-20, 10-150, 10-100, 10-50, 10-20, 50-150, 50-100, or 100-150 of the markers recited herein.

The binding partners may be antibodies, antigen-binding antibody fragments, receptors, ligands, aptamers, nucleotides and the like, provided they bind selectively to the marker being tested and do not bind appreciably to any other marker that may be present in the biological sample loaded onto the device.

The binding partners may be provided on the substrate in a predetermined spatial arrangement. A substrate, as used herein in this context, refers to a solid support to which marker-specific binding partners may be bound. The substrate may be paper or plastic (e.g., polystyrene) or some other material that is amenable to the marker measurement. The substrate may have a planar surface although it is not so limited. In some instances, the substrate is a bead or sphere.

The art is familiar with diagnostic devices and reference can be made to U.S. Pat. Nos. 7,897,356 and 7,323,143, and published US Patent Application Publication No. US 2008/0267999, and Martinez et al. PNAS, 2008, 105 (50): 19606-19611, all of which are incorporated herein by reference in their entirety.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

### EXAMPLES

### Example 1

### Materials and Methods

A library of ORFS in pDONR-223 Entry vectors (Invitrogen) was assembled. Individual clones were end-sequenced using vector-specific primers in both directions. Clones with substantial deviations from reported sequences were discarded. Entry clones and sequences are available via Addgene online. ORFS were assembled from multiple sources; including those isolated as single clones from the ORFeome 5.1 collection, those cloned from normal human tissue RNA (Ambion) by reverse transcription and subsequent PCR amplification to add Gateway sequences (Invitrogen), those cloned from templates provided by the Harvard Institute of Proteomics (HIP), and those cloned into the Gateway system from templates obtained from collaborating laboratories. The Gateway-compatible lentiviral vector pLX-Blast-V5 was created from the pLKO.1 backbone. LR Clonase enzymatic recombination reactions were performed to introduce the ORFS into pLX-Blast-V5 according to the manufacturer's protocol (Invitrogen).

### High Throughput ORF Screening

A375 melanoma cells were plated in 384-well microtiter plates (500 cells per well). The following day, cells were spin-infected with the lentivirally-packaged ORF library in the presence of 8 ug/ml polybrene. 48 hours post-infection, media was replaced with standard growth media (2 replicates), media containing 1 μM PLX4720 (2 replicates, 2 time points) or media containing 10 ug/ml blasticidin (2 replicates). After four days and 6 days, cell growth was assayed using Cell Titer-Glo (Promega) according to manufacturer instructions. The entire experiment was performed twice.

### Identification of Candidate Resistance ORFS

Raw luminescence values were imported into Microsoft Excel. Infection efficiency was determined by the percentage of duplicate-averaged raw luminescence in blasticidin selected cells relative to non-selected cells. ORFS with an infection efficiency of less than 0.70 were excluded from further analysis along with any ORF having a standard deviation of >15,000 raw luminescence units between duplicates. To identify ORFS whose expression affects proliferation, the duplicate-averaged raw luminescence of individual ORFS was compared against the average and standard deviation of all control-treated cells via the z-score, or standard score, below,

$$Z = \frac{χ - μ}{σ}$$

where x=average raw luminescence of a given ORF, p=the mean raw luminescence of all ORFS and σ=the standard deviation of the raw luminescence of all wells. Any individual ORF with a z-score >+2 or <−2 was annotated as affecting proliferation and removed from final analysis. Differential proliferation was determined by the percentage of duplicate-averaged raw luminescence values in PLX4720 (1 μM) treated cells relative to untreated cells. Subsequently, differential proliferation was normalized to the positive control for PLX4720 resistance, MEK1^{S218/222D} (MEK1^{DD}), with MEK1^{DD} differential proliferation=1.0. MEK1^{DD} normalized differential proliferation for each individual ORF was averaged across two duplicate experiments, with two time points for each experiment (day 4 and day 6). A z-score was then generated, as described above for average MEK1^{DD} normalized differential proliferation. ORFS with a z-score of >2 were considered hits and were followed up in the secondary screen.

### Secondary Screen

A375 (1.5×10³) and SKMEL28 cells (3×10³) were seeded in 96-well plates for 18 h. ORF-expressing lentivirus was added at a 1:10 dilution in the presence of 8 μg/ml polybrene, and centrifuged at 2250 RPM and 37° C. for 1 h. Following centrifugation, virus-containing media was changed to normal growth media and allowed to incubate for 18 h. Twenty-four hours after infection, DMSO (1:1000) or 10× PLX4720 (in DMSO) was added to a final concentration of 100, 10, 1, 0.1, 0.01, 0.001, 0.0001 or 0.00001 μM. Cell viability was assayed using WST-1 (Roche), per manufacturer recommendation, 4 days after the addition of PLX4720.

### Cell Lines and Reagents

Cell lines were grown in RPMI (Cellgro), 10% FBS and 1% penicillin/streptomycin. M307 was grown in RPMI (Cellgro), 10% FBS and 1% penicillin/streptomycin supplemented with 1 mM sodium pyruvate. 293T and OUMS-23 were grown in DMEM (Cellgro), 10% FBS and 1% penicillin/streptomycin. RPMI-7951 cells (ATCC) were grown in MEM (Cellgro), 10% FBS and 1% penicillin/streptomycin. Wild-type primary melanocytes were grown in HAM's F10 (Cellgro), 10% FBS and 1% penicillin/streptomycin. B-RAF^{V600E}-expressing primary melanocytes were grown in TIVA media [Ham's F-10 (Cellgro), 7% FBS, 1% penicillin/streptomycin, 2 mM glutamine (Cellgro), 100 uM IBMX, 50 ng/ml TPA, 1 mM dbcAMP (Sigma) and 1 μM sodium vanadate]. CI-1040 (PubChem ID: 6918454) was purchased from Shanghai Lechen International Trading Co., AZD6244 (PubChem ID: 10127622) from Selleck Chemicals, and PLX4720 (PubChem ID: 24180719) from Symansis. RAF265 (PubChem ID: 11656518) was a generous gift from Novartis Pharma AG. Unless otherwise indicated, all drug treatments were for 16 h. Activated alleles of NRAS and KRAS have been previously described. (Boehm, J. S. et al. Cell 129, 1065-1079 (2007); Lundberg, A. S. et al. Oncogene 21, 4577-4586 (2002)).

### Pharmacologic Growth Inhibition Assays

Cultured cells were seeded into 96-well plates (3,000 cells per well) for all melanoma cell lines; 1,500 cells were seeded for A375. Twenty-four hours after seeding, serial dilutions of the relevant compound were prepared in DMSO added to cells, yielding final drug concentrations ranging from 100 μM to 1×105 μM, with the final volume of DMSO not exceeding 1%. Cells were incubated for 96 h following addition of drug. Cell viability was measured using the WST1 viability assay (Roche). Viability was calculated as a percentage of control (untreated cells) after background subtraction. A minimum of six replicates were performed for each cell line and drug combination. Data from growth-inhibition assays were modeled using a nonlinear regression curve fit with a sigmoid dose-response. These curves were displayed and GI50 generated using GraphPad Prism 5 for Windows (GraphPad). Sigmoid-response curves that crossed the 50% inhibition point at or above 10 μM have GI50 values annotated as >10 μM. For single-dose studies, the identical protocol was followed, using a single dose of indicated drug (1 μM unless otherwise noted).

### Immunoblots and Immunoprecipitations

Cells were washed twice with ice-cold PBS and lysed with 1% NP-40 buffer [150 mM NaCl, 50 mM Tris pH 7.5, 2 mM EDTA pH 8, 25 mM NaF and 1% NP-40] containing 2× protease inhibitors (Roche) and 1× Phosphatase Inhibitor Cocktails I and II (CalBioChem). Lysates were quantified (Bradford assay), normalized, reduced, denatured (95° C.) and resolved by SDS gel electrophoresis on 10% Tris/Glycine gels (Invitrogen). Protein was transferred to PVDF membranes and probed with primary antibodies recognizing pERK1/2 (T202/Y204), pMEK1/2 (S217/221), MEK1/2, MEK1, MEK2, V5-HRP (Invitrogen; (1:5,000), Rac1, CDC42, RAC1-GTP, CDc42-GTP, and CyD1. After incubation with the appropriate secondary antibody (anti-rabbit, anti-mouse IgG, HRP-linked; 1:1,000 dilution, Cell Signaling Technology or anti-goat IgG, HRP-linked; 1:1,000 dilution; Santa Cruz), proteins were detected using chemiluminescence (Pierce). Immunoprecipitations were performed overnight at 4° C. in 1% NP-40 lysis buffer, as described above, at a concentration of 1 μg/μl total protein. Antibody: antigen complexes were bound to Protein A agarose (25 μL, 50% slurry; Pierce) for 2 hrs. at 4° C. Beads were centrifuged and washed three times in lysis buffer and eluted and denatured (95° C.) in 2× reduced sample buffer (Invitrogen). Immunoblots were performed as above. Phospho-protein quantification was performed using NIH Image J.

### An ORF-Based Functional Screen Identifies GEFs as Drivers of Resistance to B-RAF Inhibition.

To identify proteins capable of circumventing RAF inhibition, about 15,000 ORF clones were assembled and stably expressed in A375, a B-RAF^{V600E} malignant melanoma cell line that is sensitive to the RAF kinase inhibitor PLX4720 (Tsai, J. et al. Proc. Natl Acad. Sci. USA 105, 3041-3046 (2008)). ORF expressing cells treated with 1 μM PLX4720 were screened for viability relative to untreated cells and normalized to an assay-specific positive control, MEK1^{S218/222D} (MEK1^{DD}) (Emery, C. M. et al. Proc. Natl Acad. Sci. USA 106, 20411-20416 (2009)). ORFS conferring resistance at levels exceeding 2.5 standard deviations from the mean were selected for follow-up analysis. A number of the candidate ORFS were GEFs, underscoring the potential of this class of proteins to impact resistance pathways. Resistance effects were validated across a multi-point PLX4720 drug concentration scale in the B-RAF^{V600E} cell line A375. The GEFs ARHGEF2, ARHGEF3, ARHGEF9, ARHGEF19, IQSEC1, MCF2L, NGEF, PLEKHG3, PLEKHG5, PLEKHG6, TBC1D3G, SPATA13, and VAV1 emerged as top candidates. These ORFS shifted the PLX4720 GI₅₀ by 2.5-30+ fold without affecting viability.

### GEF-Expressing B-RAF^{V600E} Cell Line Clones Exhibit Resistance to MEK Inhibitors.

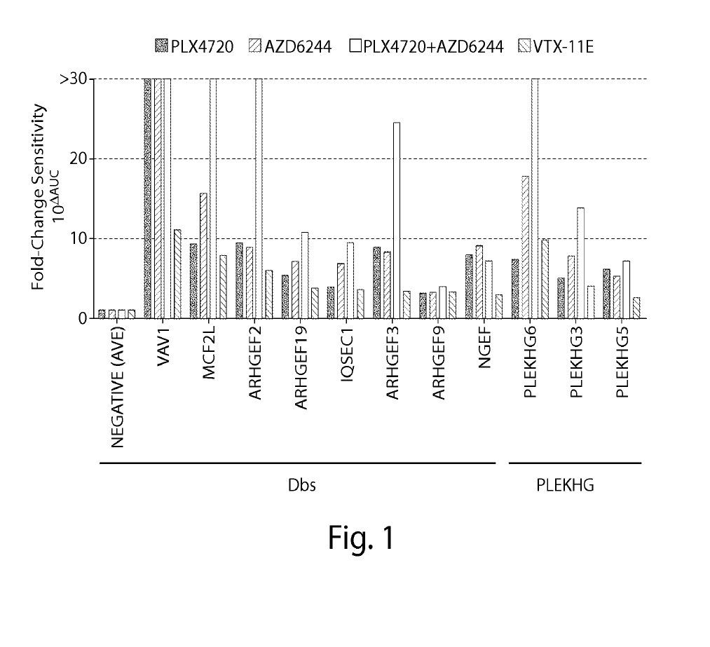

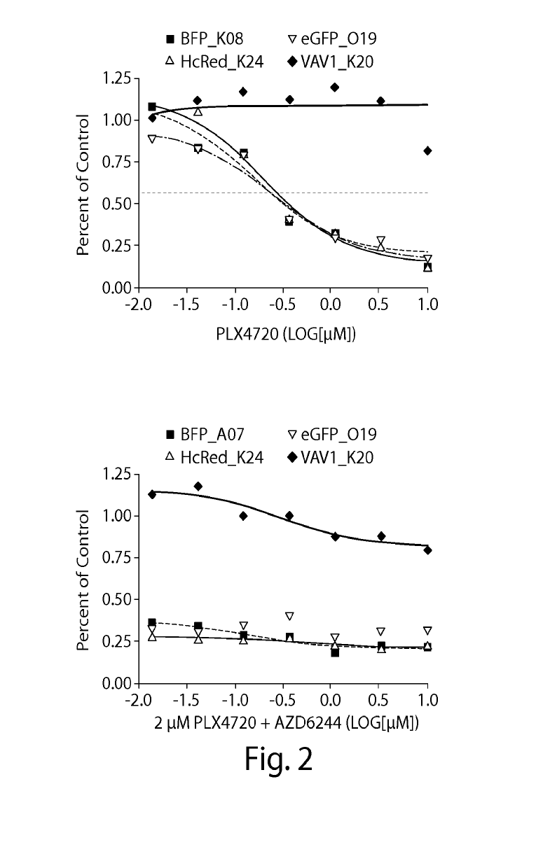

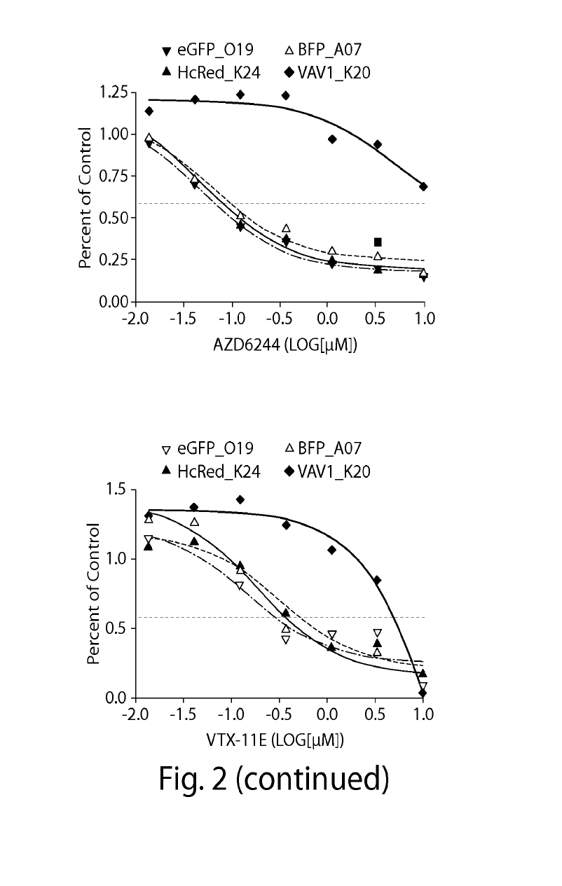

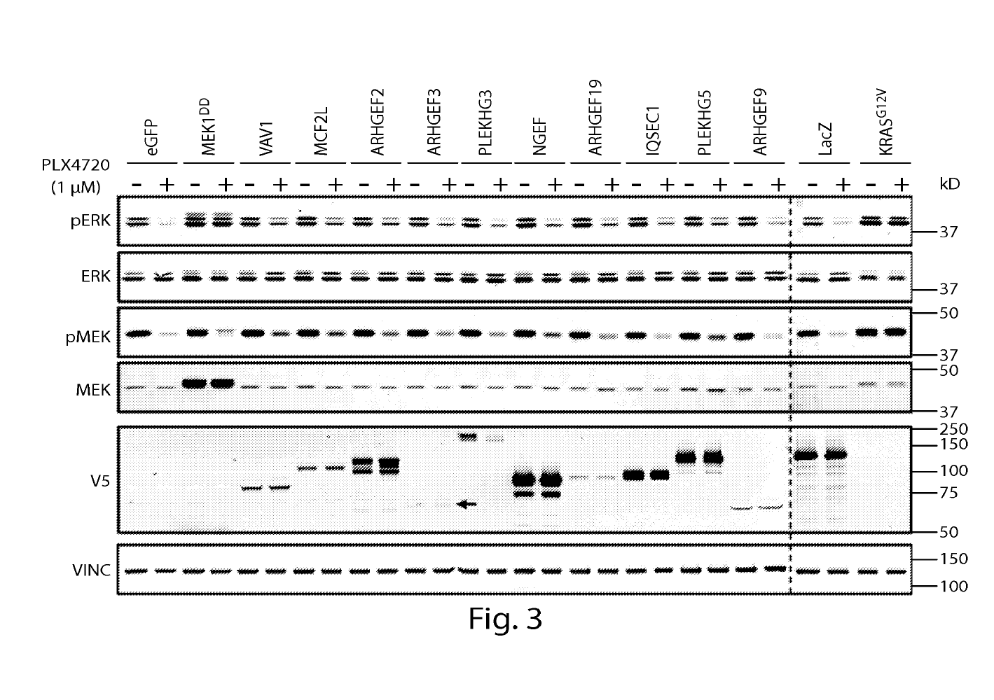

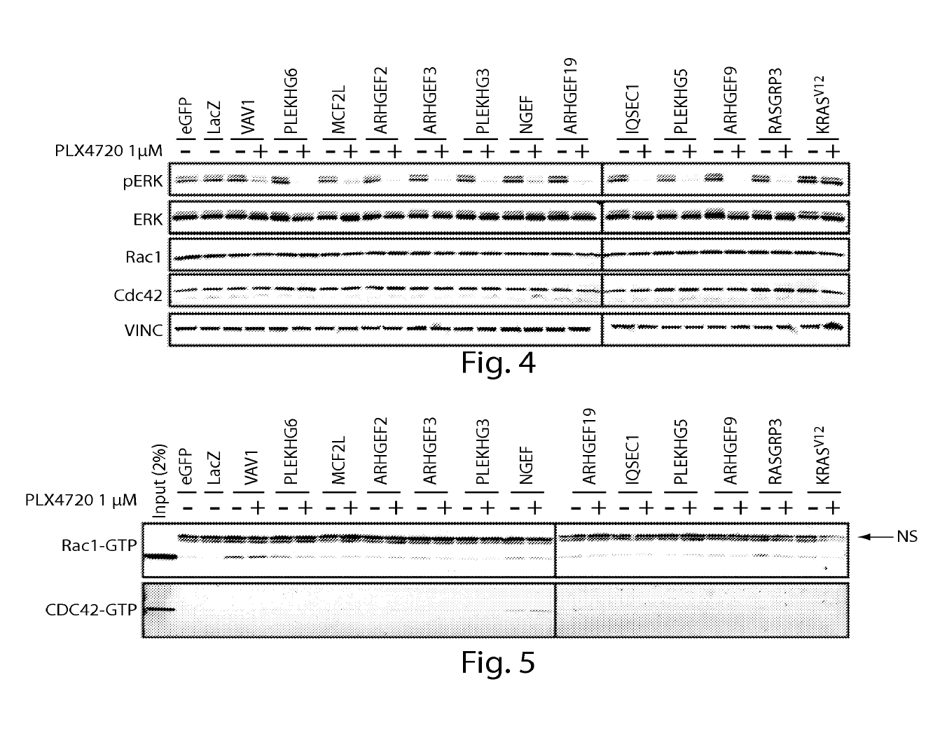

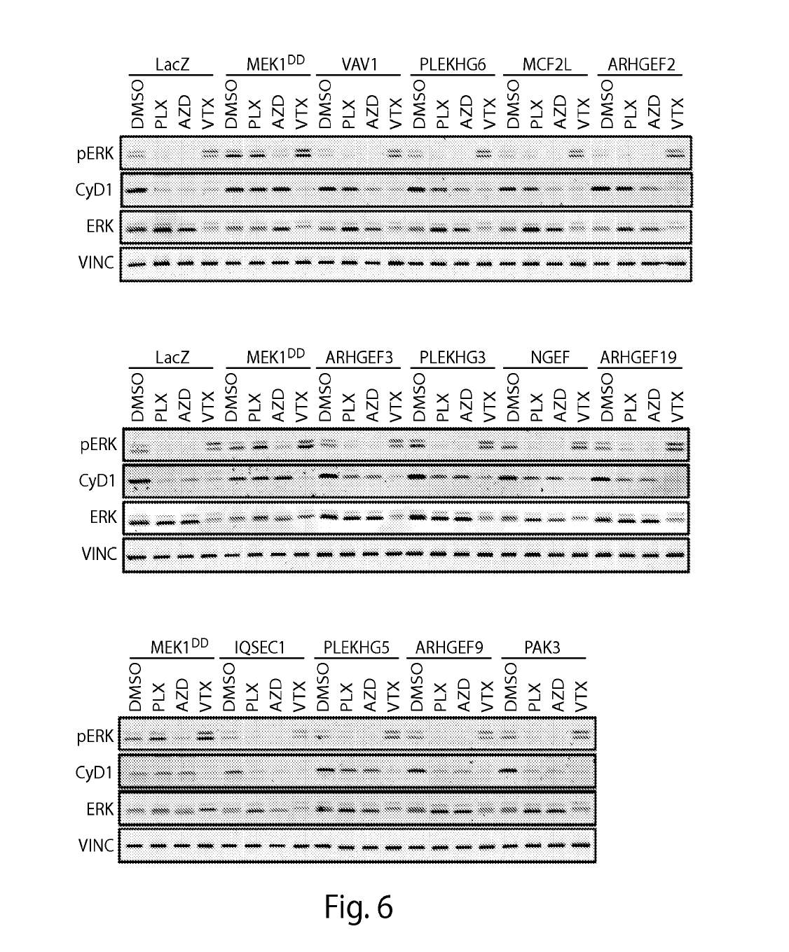

Whether GEF-expressing cancer cells remain sensitive to MAPK pathway inhibition at a target downstream of RAF was analyzed. The A375 cell line which is sensitive to AZD6244, a combination of PLX4720 and AZD6244, and VTZ-11E was transfected with GEF ORFS and then cultured in the presence of these inhibitors. Ectopic GEF expression conferred decreased sensitivity to the MEK inhibitor AZD6244, the combination of PLX4720 and AZD6244, and to VTX-11E, suggesting that GEF expression alone was sufficient to induce this phenotype (FIG. 1 and FIG. 2).

### Example 2

### Methods

### Lentiviral Expression Library

The genesis, cloning, sequencing and production of the Broad-Institute/Center for Cancer Systems Biology Lentiviral Expression Library has been described previously [ref 17]. All ORFS described in this manuscript were expressed from pLX304, a lentiviral expression vector that encodes a C-terminal V5-epitope tag, a blasticydin resistance gene and drives ORF expression from a CMV-promoter. All clones described in this manuscript are publicly available via members of the ORFeome collaboration (orfeomecollaboration.org).

### Genome Scale ORF Resistance Screens

A375 were robotically seeded into 384-well white walled, clear-bottom plates in RPMI-1640 (cellgro) supplemented with 10% FBS and 1% Penicillin/Streptomycin. The cloning, sequencing and production of the Broad-Institute/Center for Cancer Systems Biology Lentiviral Expression Library17 was arrayed on 47×384 well plates, permitting robotic transfer of virus to cell plates. Cell plates were randomly divided into 6 treatment arms in duplicate: DMSO, PLX4720, AZD6244, PLX4720+AZD6244, VRT11e or a parallel selection arm (blasticydin). Twenty-four hours after seeding, polybrene was added directly to cells (7.5 μg/ml final concentration), followed immediately by robotic addition of the CCSB/Broad Institute virus collection (3 μL/well) and centrifuged at 2250 RPM (1,178×g) for 30 min. at 37° C. Following a 24 hr. incubation at 37° C. (5% CO2), media and virus was aspirated and replaced with complete growth media or media containing blasticydin (10 μg/ml) to select for ORF expressing cells and to determine infection efficiency. Forty-eight hours after media change, unselected (no blasticydin) cells were treated with DMSO (vehicle control) or MAPK pathway inhibitors to a final concentration of 2 μM (PLX4720, VRT11e) or 200 nM (AZD6244). Identical concentrations used for single agent PLX4720 and AZD6244 treatment were used for combined PLX4720/AZD6244 treatment and single-agent inhibitors were balanced with DMSO such that all wells contained 0.033% DMSO. Four days (96 hrs.) after drug addition, cell viability was assessed via robotic addition of CellTiterGlo (1:6 dilution) followed by 10 min. orbital agitation at room temperature and subsequent quantification (EnVision Multilabel Reader, Perkin Elmer). Primary screens were performed in 16 individual batches in which 2-3 viral stock plates were screened per batch against all compounds.

Identification of Resistance Candidates from Primary Screening Data

Following quantification of cell viability, duplicate luminescence values were averaged per ORF within each treatment condition. Percent rescue capability of each ORF was determined by dividing the average luminescence value in drug by the average luminescence value in DMSO. Subsequent percent rescue values were normalized within screening plates using the plate average and standard deviation to generate a z-score/standard score of percent rescue, herein referred to as the ‘rescue score’. To calculate infection efficiency of each ORF, luminescence values in the presence of blasticydin were normalized to the average luminescence in DMSO and expressed as a percentage. ORF-mediated effects on cell viability were assessed by taking the average luminescence value for each ORF in DMSO and normalizing each value to the plate average and standard deviation (z-score). To identify candidate resistance genes, first all wells that had an infection efficiency of less than 65% were filtered out. To eliminate genes with significant effects on cellular growth in the absence of drug treatment, genes that had a z-score in DMSO of greater than 2.0 or less than −2.0 were then filtered out. Additionally wells from further analysis that showed a replicate variability (in DMSO) of greater than 29.15% (equivalent to >2 standard deviations from the average replicate variability) were eliminated. Following this initial filtering, 14,457 genes remained for subsequent analysis. Within each drug treatment condition, wells showing replicate variability of >2 standard deviations from the mean variability per drug were eliminated from further analysis. Finally, genes showing a z-score of percent rescue of greater than 2.5 were nominated as resistance gene candidates. Neutral control genes (19) were nominated from primary screening data by identifying genes across virus plates and screening batches with 1) high infection efficiency (>98.5%), 2) minimal effects on baseline cell growth (z-score of viability in DMSO between −0.5 to 0.5) and 3) a rescue score (z-score of percent rescue) <0.25 (e.g. no effect on drug sensitivity or resistance). DNA encoding candidates (169), negative controls (eGFP, n=9; HcRed, n=15; Luciferase, n=16) positive controls (MEK1 DD, KRASG12V, MAP3K8/COT) and neutral controls (19) were isolated from the CCSB/Broad expression collection and used to create a validation viral stock distinct from that used in the primary screens.

### Drug Sensitivity Curves in A375 Expressing Candidate ORFS

A375 were seeded, infected and drug treated exactly as in primary screens using 4 μl of validation viral stock and concentrations of inhibitors ranging from 10 μM to 100 nM in half-log increments. For combinatorial PLX4720/AZD6244 treatment, a fixed dose of PLX4720 (2 μM) was combined with AZD6244 in doses ranging from 10 μM to 100 nM in half-log increments. Viability was assessed as in the primary screen. Resulting luminescence for each ORF was normalized to luminescence in DMSO (% rescue) for each drug and drug concentration. Resulting sensitivity curves for each ORF were log transformed and the area under the curve (AUC) calculated using Prism GraphPad software. Resulting AUC for each candidate and control ORF/drug combination were normalized to that of the negative and neutral controls using a z-score (described above). ORFS yielding a z-score of >1.96 (p<0.05) were considered to be validated candidates in this cell line.

### Validation Screens in Additional BRAFV600E Cell Lines

Validation screening in additional BRAFV600E melanoma cell lines was performed exactly as in the primary screen, but cell lines were empirically optimized for seeding density and viral dilution. Due to sensitivity of these cell lines to polybrene and virus exposure, all cell lines except for WM266.4 were treated with polybrene and virus, spun for 1 hr. at 2250 RPM (1,178×g) followed immediately by complete virus/media removal and change to complete growth media. WM266.4 were treated with polybrene and virus, spun for 30 min. at 2250 RPM (1,178×g) and incubated for 24 hours before virus/media removal and change to complete growth media 24 hours after infection. For experimental determination of infection efficiency, blasticydin (5 μg/ml) was added 24 hrs. after media change. All drug treatments and viability measurements were performed as in primary screens. Resulting luminescence values were normalized to DMSO (percent of DMSO or ‘percent rescue’). Resulting percent rescue was normalized to the mean and standard deviation of all negative and neutral controls to yield a z-score of percent rescue, herein referred to as the “rescue score”. Genes with a rescue score of >4 in at least one drug condition across at least 2 independent cell lines were considered to have validated. “Composite rescue scores” were derived by summing the rescue scores of each gene across all drugs and cell lines. Average composite rescue scores for each protein class were generated by taking the average composite rescue score of all genes within a given protein class.

pERK and V5 Immunoassays

For analysis of ERK phosphorylation, A375 were seeded at 1500 cells/well in black walled, clear bottomed, 384-well plates, virally transduced with all candidates and controls and treated with PLX4720, AZD6244 and combinatorial PLX4720/AZD6244 exactly as in the primary resistance screens. Eighteen hours after drug treatment, media was removed and cells were fixed with 4% formaldehyde and 0.1% Triton X-100 in PBS for 30 minutes at room temperature. Following removal of fixation solution, cells were washed once with PBS and blocked in blocking buffer (LiCOR) for 1 hour at room temperature with shaking. After removal of blocking buffer, primary antibody against ERK phosphorylated at Thr202/Tyr204 (Sigma, 1:2000) in LiCOR blocking buffer containing 0.1% Tween-20 and incubated for 18 hours at 4° C. with shaking. Antibody was removed and wells were washed thrice with 0.1% Tween-20 in water followed by incubation in secondary antibody (IRDye 800CW LiCOR, 1:1,200) and dual cellular stains, including Sapphire700 (LiCOR, 1:1000) and DRAQ5 (Cell Signaling Technology, 1:10,000), all diluted in LiCOR blocking buffer (no detergent) and incubated for 1 hour at room temperature with shaking. Secondary antibody/cell stain was removed and washed thrice with 0.1% Tween-20 in water followed by a single wash in PBS. PBS was removed and plates were dried for 10 minutes at room temperature in the dark followed immediately by imaging on an Odyssey CLx Infrared Scanner. For pERK and cellular stain, background was subtracted based on signal observed in control wells containing only secondary antibody in blocking buffer. Total pERK signal was normalized to total cellular stain for each ORF in each drug condition. Resulting values were subsequently normalized to DMSO (percent of DMSO) for each ORF per drug condition

V5 immunostaining for ectopic ORF expression was performed as described for the ERK phosphorylation assay, above. Briefly, cells were seeded at 3000-4000 cells/well and infected in parallel to with validation screens. Seventy-two hours after infection, cells were fixed, blocked and stained as described for the pERK assay, instead using an antibody directed against the V5 epitope (1:5,000, Invitrogen). Subsequent washes, secondary antibody incubations and total cellular staining protocol were identical to those described for the pERK assay, above. V5 and cellular stain (DRAQ5/Sapphire700) intensity were quantified as above, background signal subtracted (determined by signal intensity in uninfected wells with no V5 epitope and stained with secondary antibody, only) and V5 signal intensity normalized to cellular stain intensity.

### Detection of GPCR-Mediated Cyclic AMP Production

HEK293T cells were seeded at a density of 2.5×10⁵ cells/well in 12-well plates. Twenty-four hours after seeding, cells were transfected with 250 ng of the indicated ORF (pLX304 expression vector) using 3 μl of Fugene6 (Promega) transfection reagent. Forty-seven hours after transfection, cells were treated either with DMSO (1:1000) or IBMX (30 μM). In addition, forskolin (10 μM) and 100 M IBMX were added as positive controls for indicated time. Cells were subsequently lysed in triton x-100 lysis buffer (Cell Signaling Technology) and resulting lysates split for cAMP ELIZA (Cell Signaling Technology) or parallel western blot analysis. cAMP ELIZA was performed exactly per the manufacturers recommended protocol. Following quantification the inverse absorbance was calculated and normalized to that of negative control ORFS.

### Identification of Cyclic AMP Response Elements in Candidate Resistance Genes

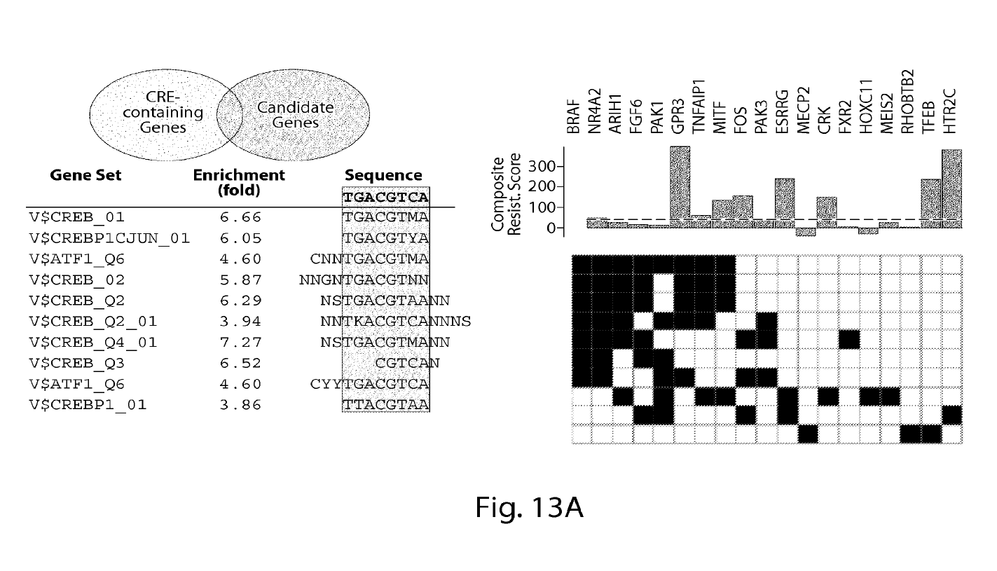

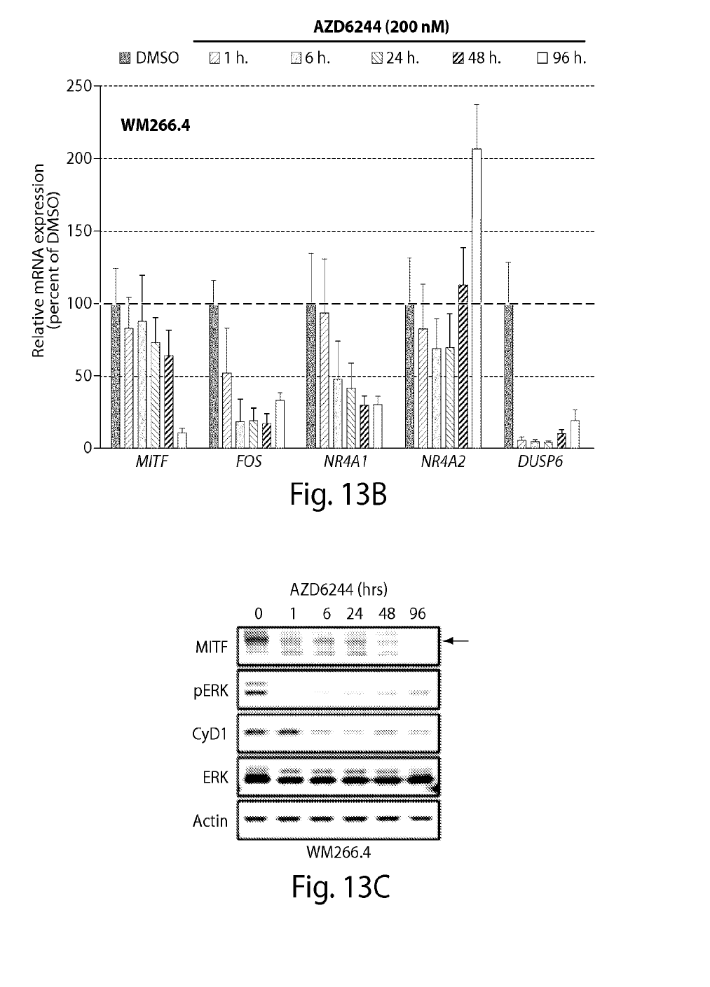

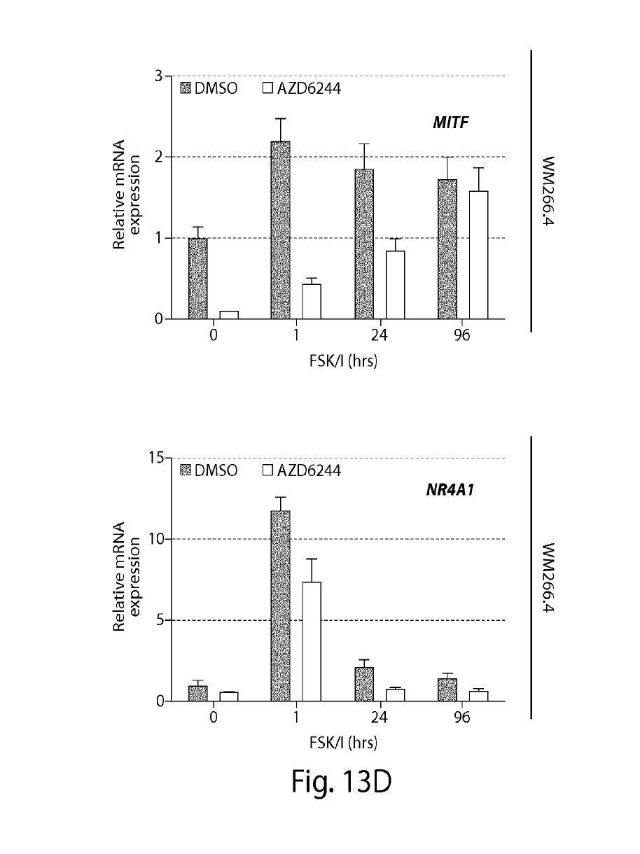

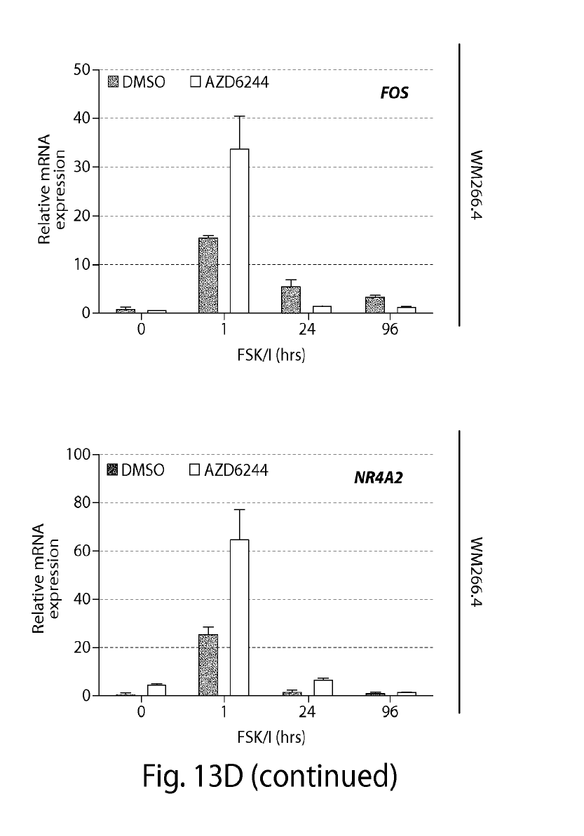

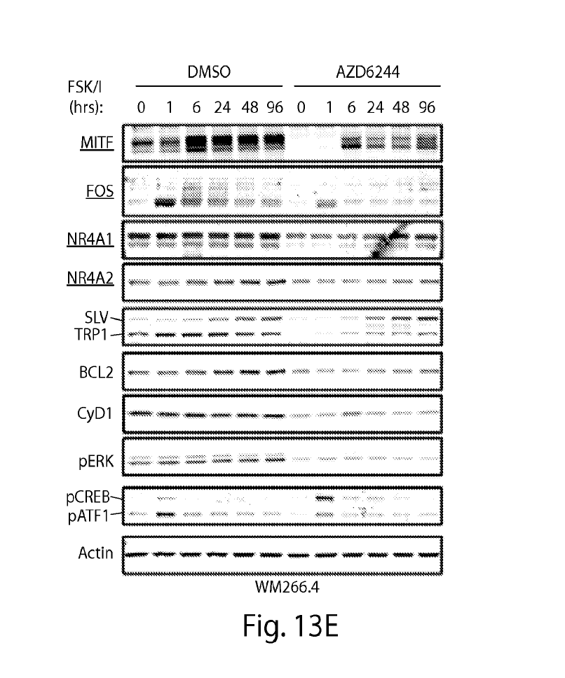

Gene sets containing genes that share a common CREB1, ATF1, ATF2 or JUN DNA response element within +/−2 kb of their transcriptional start site (as defined by TRANSFAC, version 7.4. TRANSFAC (available at the gene-regulation website) were identified and downloaded from the MSigDB website (FIG. 13(a), available at the Broad Institute website). CRE-containing genes present in individual gene sets were subsequently identified within the group of screened ORFS and within the group of candidate/neutral control ORFS. The ratio of CRE-containing genes to screened genes was compared to the ratio of CRE-containing genes to candidate/neutral control genes across gene sets. A p value for the observed enrichment of CRE-containing genes in the candidate genes over the expected representation within the screening set was calculated using Pearson's chi-squared test.

### Cell Lines and Reagents

A375, SKMEL28, UACC62, COLO-679, SKMEL5 and WM983b were all grown in RPMI-1640 (Cellgro), 10% FBS and 1% penicillin/streptomycin. WM88, G361, WM266.4, COLO-205 and 293T were all grown in DMEM (Cellgro), 10% FBS and 1% penicillin/streptomycin. Primary melanocytes were grown in TICVA media [Ham's F-10 (Cellgro), 7% FBS, 1% penicillin/streptomycin, 2 mM glutamine (Cellgro), 100 uM IBMX, 50 ng/ml TPA, 1 mM dbcAMP (Sigma) and 1 μM sodium vanadate]. Primary melanocytes seeded in TICVA media were cAMP-starved by (24 hours after seeding) washing twice with PBS and replacing media with Ham's F-10 containing 10% FBS and 1% penicillin/streptomycin for 96 hours (cAMP starved). Control (+cAMP) cells were treated at the time of media change with 1 mM dbcAMP (Sigma) and IBMX (100 μM). AZD6244 (PubChem ID: 10127622) was purchased from Selleck Chemicals, PLX4720 (PubChem ID: 24180719) was purchased from Symansis and VRT11e was synthesized by contract based on its published structure19. Forskolin, IBMX (3-Isobutyl-1-methylxanthine) and α-MSH (α-melanocyte stimulating hormone) were purchased from Sigma. Panobinostat/LBH-589 was purchased from BioVision, Vorinostat/SAHA and Entinostat/MS-275 from were purchased from Cayman Chemical.

### Pharmacologic Growth Inhibition Assays

Melanoma cell lines were seeded into 384-well, white-walled, clear bottom plates at the following densities; A375, 500 cells/well; SKMEL19, 1500 cells/well; SKMEL28, 1000 cells/well; UACC62, 1000 cells/well; WM266.4, 1800 cells/well; G361, 1200 cells/well, COLO-679, 2000 cells/well; SKMEL5, 2000 cells/well). Twenty-four hours after seeding, serial dilutions of the relevant compound were prepared in DMSO to 1000× stocks. Drug stocks were then diluted 1:100 into appropriate growth media and added to cells at a dilution of 1:10 (lx final), yielding drug concentrations ranging from 100 μM to 1×10-5 μM, with the final volume of DMSO not exceeding 1%. When indicated, forskolin (10 μM), IBMX (100 μM), dbcAMP (100 μM) were added concurrent with MAPK-pathway inhibitors. Cells were incubated for 96 h following addition of drug. Cell viability was measured using CellTiterGlo viability assay (Promega). Viability was calculated as a percentage of control (DMSO treated cells). A minimum of six replicates were performed for each cell line and drug combination. Data from growth-inhibition assays were modeled using a nonlinear regression curve fit with a sigmoid dose-response. These curves were displayed and GI50 generated using GraphPad Prism 5 for Windows (GraphPad). Sigmoid-response curves that crossed the 50% inhibition point at or above 1.0 μM or 10.0 μM have GI50 values annotated as >1.0 μM or >10.0 μM, respectively. For single-dose studies, WM266.4 were seeded at 5,000 cells/well in 96-well, white-walled, clear bottom plates and the identical protocol (above) was followed, using a single dose of indicated drug.

Low-Throughput ORF and shRNA Expression

Indicated ORFS were expressed from pLX-304 (Blast, V5) lentiviral expression plasmids, whereas shRNAs were expressed from pLKO.1. shRNAs and controls are available through The RNAi Consortium Portal (Broad Institute Website) and are identifiable by their clone ID: shLuc (TRCN0000072243), shMITF_{—}492 (TRCN0000329869), shMITF_{—}573 (TRCN0000019123), shMITF_{—}956 (TRCN0000019120) and shMITF_{—}3150 (TRCN0000019119). For lentiviral production, 293T cells (1.0×106 cells/6-cm dish) were transfected with 1 μg of pLX-Blast-V5-ORF or pLKO.1-shRNA, 900 ng Δ8.9 (gag, pol) and 100 ng VSV-G using 6 μl Fugene6 transfection reagent (Promega). Viral supernatant was harvested 72 h post-transfection. WM266.4 were infected at a 1:10-1:20 dilution (ORFS) or 1:100 dilution (shRNA) of virus in 6-well plates (2.0×105 cells/well, for immunoblot assays) or 96-well plates (3.0×103, for cell growth assays) in the presence of 5.5 μg/ml polybrene and centrifuged at 2250 RPM for 60 min. at 37° C. followed immediately by removal of media and replacement with complete growth media. Seventy-two hours after infection, drug treatments/pharmacological perturbations were initiated (see below).

### CREB1 and MITF Mutagenesis, Generation of A-CREB

Wild-type CREB1 (Isoform B, NM_{—}134442.3) was obtained through the Broad Institute RNAi Consortium, a member of the ORFeome Collaboration (available at the orfeomecollaboration website). Arginine 301 of CREB was mutated to Leucine yielding CREBR301L (equivalent to CREBR287L in isoform A) and arginine 217 of MITF-m29 was deleted using the QuikChange Lightning Mutagenesis Kit (Agilent), performed in pDonor223 (Invitrogen). CREBR301L and MITF-mR217Δ was transferred into pLX304 using LR Clonase (Invitrogen) per manufacturer's recommendation. The A-CREB cDNA32 was synthesized (Genewiz) with flanking Gateway recombination sequences, recombined first into pDonor223 and subsequently into pLX304 as described for MITF and CREB1 mutant cDNAs.

### Quantitative RT/PCR

mRNA was extracted from WM266.4 using the RNeasy kit (Qiagen) and homogenized using the Qiashredder kit (Qiagen). Total mRNA was used for subsequent reverse transcription using the SuperScript III First-Strand Synthesis SuperMix (Invitrogen). 5 μl of reverse-transcribed cDNA was used for quantitative PCR using SYBR Green PCR Master Mix and gene-specific primers, in quadruplicate, using an ABI PRISM 7900 Real Time PCR System. Primers used for detection were as follows; NR4A2 forward: 5′-GTT CAG GCG CAG TAT GGG TC-3′ (SEQ ID NO: 7); NR4A2 reverse: 5′-AGA GTG GTA ACT GTA GCT CTG AG-3′ (SEQ ID NO: 8); NR4A1 forward: 5′-ATG CCC TGT ATC CAA GCC C-3′ (SEQ ID NO: 9); NR4A1 reverse: 5′-GTG TAG CCG TCC ATG AAG GT-3′ (SEQ ID NO: 10); DUSP6 forward: 5′-CTG CCG GGC GTT CTA CCT-3′ (SEQ ID NO: 11); DUSP6 reverse: 5′-CCA GCC AAG CAA TGT ACC AAG-3′ (SEQ ID NO: 12); MITF forward: 5′-TGC CCA GGC ATG AAC ACA C-3′ (SEQ ID NO: 13); MITF reverse: 5′-TGG GAA AAA TAC ACG CTG TGA G-3′ (SEQ ID NO: 14); FOS forward: 5′-CAC TCC AAG CGG AGA CAG AC-3′ (SEQ ID NO: 15); FOS reverse: 5′-AGG TCA TCA GGG ATC TTG CAG-3′ (SEQ ID NO: 16); TBP forward: 5′-CCC GAA ACG CCG AAT ATA ATC C-3′ (SEQ ID No: 17); TBP reverse: 5′-GAC TGT TCT TCA CTC TTG GCT C-3′ (SEQ ID NO: 18). Relative expression was determined using the comparative CT method (Applied Biosystems).

### Immunoblots and Antibodies

Adherent cells were washed once with ice-cold PBS and lysed passively with 1% NP-40 buffer [150 mM NaCl, 50 mM Tris pH 7.5, 2 mM EDTA pH 8, 25 mM NaF and 1% NP-40] containing 2× protease inhibitors (Roche) and 1× Phosphatase Inhibitor Cocktails I and II (CalBioChem). Lysates were quantified (Bradford assay), normalized, reduced, denatured (95° C.) and resolved by SDS gel electrophoresis on 4-20% Tris/Glycine gels (Invitrogen). Resolved protein was transferred to nitrocellulose or PVDF membranes, blocked in LiCOR blocking buffer and probed with primary antibodies recognizing MITF (C5), Cyclin D1 (Ab-3) (1:400; Thermo Fisher Scientific/Lab Vision), pERK1/2 (Thr202/Tyr204; 1:5,000; Sigma), SLVR (1:500; Sigma), vinculin (1:5000; Sigma), pMEK1/2 (S217/221), MEK1/2, FOS, pCREB (Ser133), CREB (1:1,000; Cell Signaling Technology), β-Actin (1:20,000; Cell Signaling Technology), V5 epitope (1:5,000; Invitrogen), BCL2 (C-2), TRP1 (G-17), Melan-A (A103), NR4A1/Nur77 (M-210), NR4A2/Nurr1 (N-20), SOX10 (N-20) (1:200; Santa Cruz). After incubation with the appropriate secondary antibody (anti-rabbit, anti-mouse or anti-goat IgG, IRDye-linked; 1:15,000 dilution; IRDye 800CW, 1:20,000 IRDye 680LT, LiCOR), proteins were imaged using an Odyssey CLx scanner (LiCOR).

Lysates from tumor and matched normal skin were generated by mechanical homogenization of tissue in RIPA [50 mM Tris (pH 7.4), 150 mM NaCl, 1 mM EDTA, 0.1% SDS, 1.0% NaDOC, 1.0% Triton X-100, 25 mM NaF, 1 mM NA3VO4] containing protease and phosphatase inhibitors, as above. Subsequent normalization and immunoblots were performed as above.

### Quantification of Melanin Content in Primary Melanocytes

NP40-insoluable material from primary melanocytes harvested in NP40-lysis buffer (see ‘Immunoblots and antibodies’, above) were pelleted and isolated from residual cellular lysates. Based on prior work49, pigmented pellets were re-suspended in 50 μl of 1 M NaOH at room temperature and absorbance quantified at 405 nM. Resulting absorbance was background subtracted and normalized to baseline control.

### Expression Profiling of Melanoma Cancer Cell Lines

An oligonucleotide microarray analysis was carried out using the GeneChip Human Genome U133 Plus 2.0 Affymetrix expression array (Affymetrix, Santa Clara, Calif.). Samples were converted to labeled, fragmented, cRNA per the Affymetrix protocol for use on the expression microarray. All expression arrays are available on the Broad-Novartis Cancer Cell Line Encyclopedia data portal at broad institute.org/ccle/home.

### Biopsied Melanoma Tumor Material

Biopsied tumor material consisted of discarded and de-identified tissue that was obtained with informed consent and characterized under protocol 02-017 (paired samples, Massachusetts General Hospital). For paired specimens, ‘on-treatment’ samples were collected 10-14 days after initiation of PLX4032 treatment.

### Results

### Defining the Spectrum of Resistance to MAPK Pathway Inhibitors

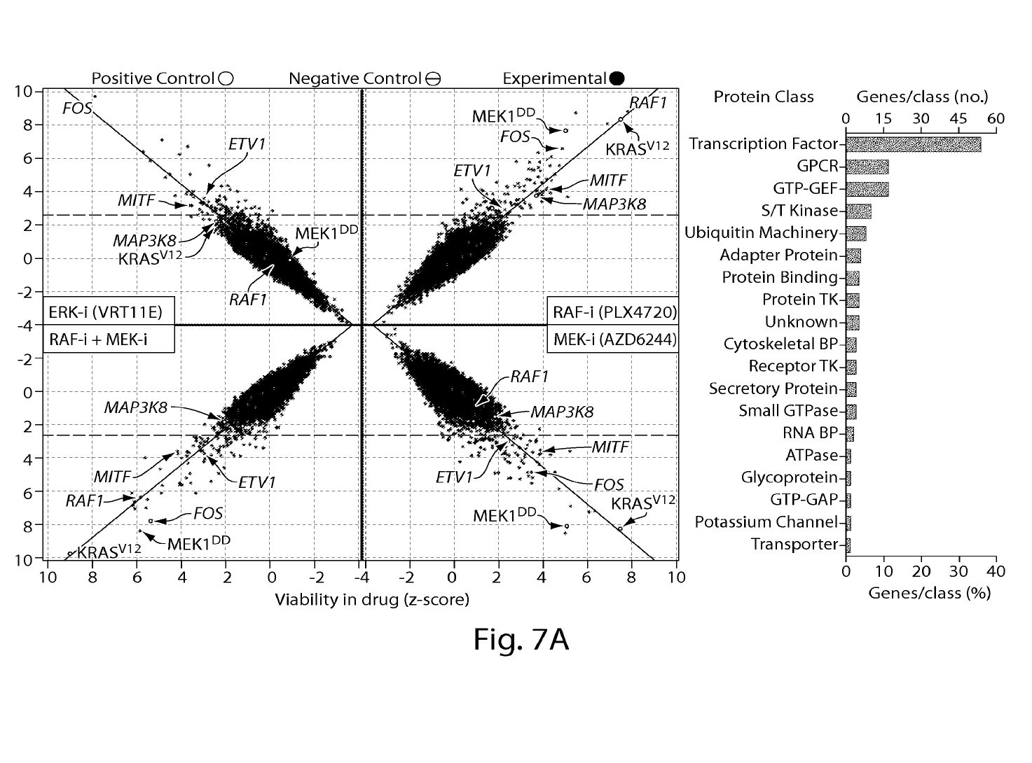

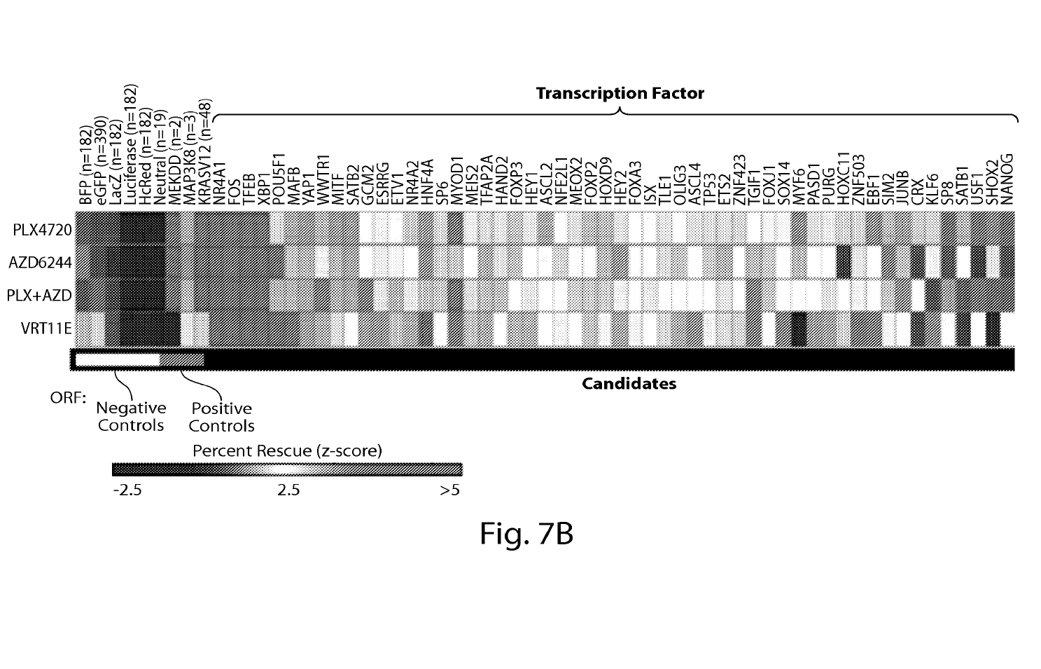

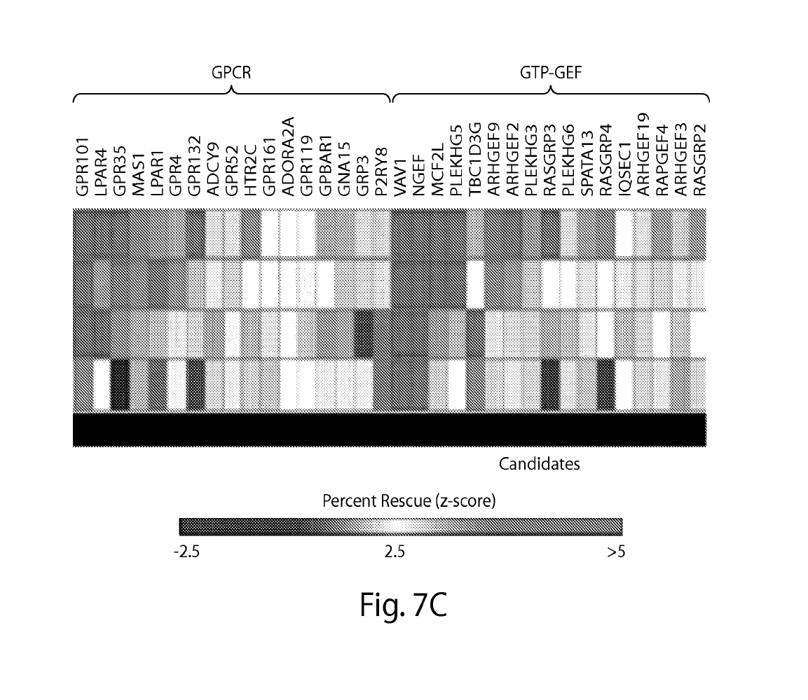

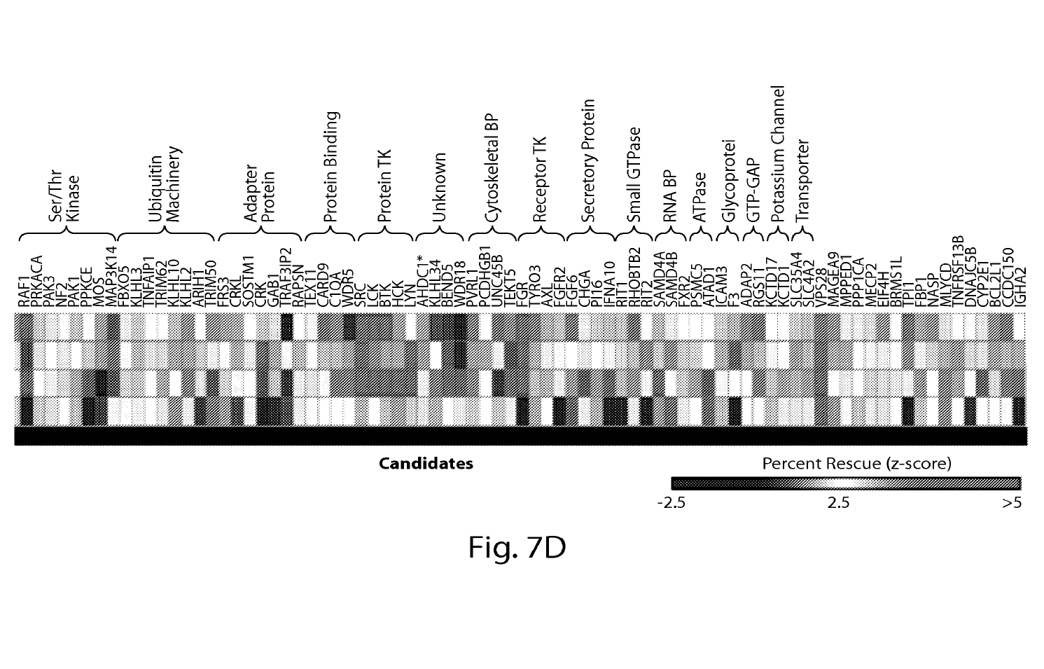

To achieve ‘global’ characterization of genes whose up-regulation is sufficient to confer resistance to MAPK pathway inhibition, a collection [ref. 17] of 15,906 human open reading frames (ORFS) was expressed in a BRAF^{V600E} melanoma cell line (A375) that is dependent on RAF/MEK/ERK signaling for growth [ref. 11 and 18]. The effect of each gene on the sensitivity of A375 cells to small-molecule inhibitors, targeting RAF (RAF-i; PLX4720), MEK (MEK-i; AZD6244), ERK¹⁹ (ERK-i; VTX11e) and a combination of RAF and MEK (RAF/MEK-i; PLX4720/AZD6244) (FIG. 7A, left panel) was determined. In this experiment, 14,457 genes (90.9%, FIG. 7A, left panel) passed empirically optimized thresholds for infection efficiency, replicate variation and effects on baseline cell growth. 169 genes (1.16%) were identified whose expression conferred resistance to at least one MAPK-pathway inhibitor, as determined by a standardized rescue score (z-score) that exceeded 2.5 (FIGS. 7B-D).

The near genome-scale scope of these experiments (13,384 unique human genes) enabled identification of diverse resistance effectors (FIG. 7A, right panel) including several canonical MAPK signaling components whose overexpression may phenocopy pathway activation. Examples included previously identified genes (KRAS^{G12V}, MEK1^{S218/222D}, RAF1, FGR, AXL and COT/MAP3K8) [refs. 20-23] and unreported genes including receptor tyrosine kinases (FGFR2), RAS-guanine exchange factors (RASGRP2/3/4) and MAP3-kinases (MOS), all of which activate ERK. Numerous genes that may implicate previously unrecognized MAPK inhibitor resistance mechanisms were also identified, including modifiers of “stem-ness” (POU5F4/OCT4, NANOG), ubiquitin pathway components (KLHL-family members, TR/M-family members), non-Ras guanine exchange factors (VAV1, other DBS and PLEKHG family members) and secreted factors (FGF6, IFNA10) (FIGS. 7A-D). Several well-characterized ERK-regulated transcription factors (TFs) not previously implicated in resistance to MAPK inhibitors, were also identified, including FOS, JUNB, ETS2 and ETV1 (FIGS. 7B-D). These results suggested that systematic resistance screens may nominate “membrane-to-nucleus” signaling networks capable of promoting resistance to MAPK-pathway inhibition.

### Comprehensive Phenotypic Characterization of Candidate Resistance Genes Identifies Broadly Validating Protein Classes.

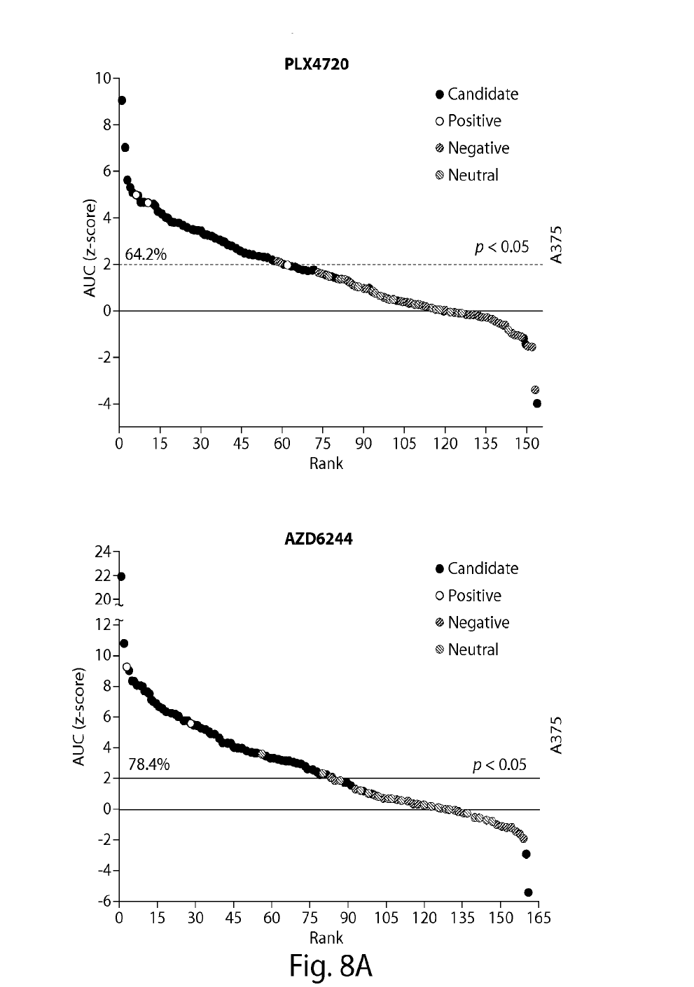

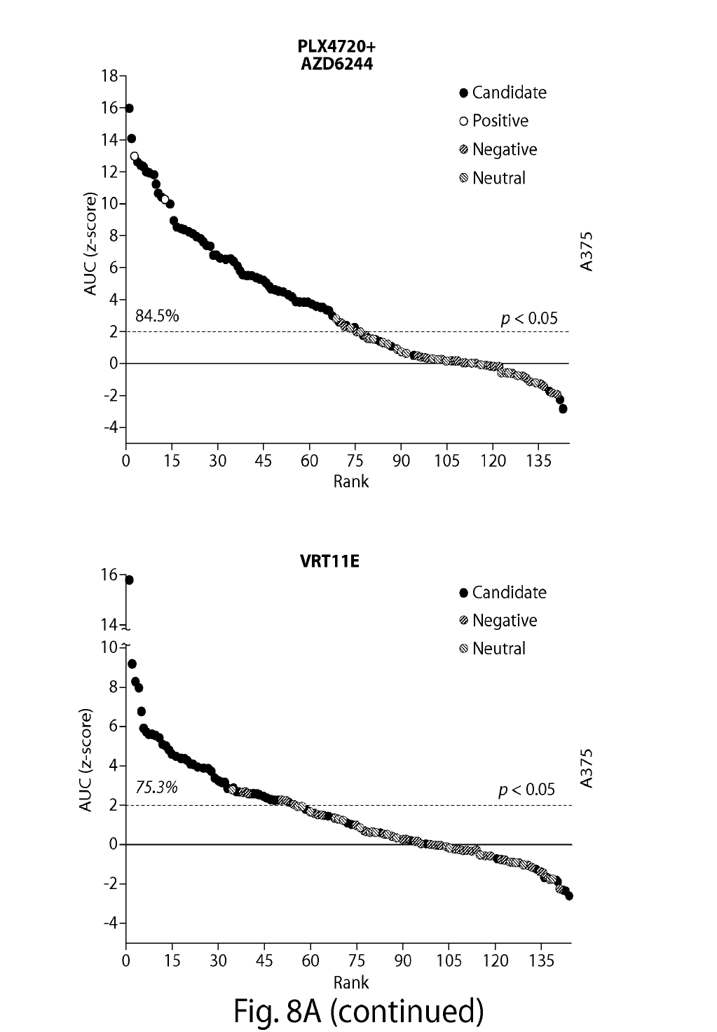

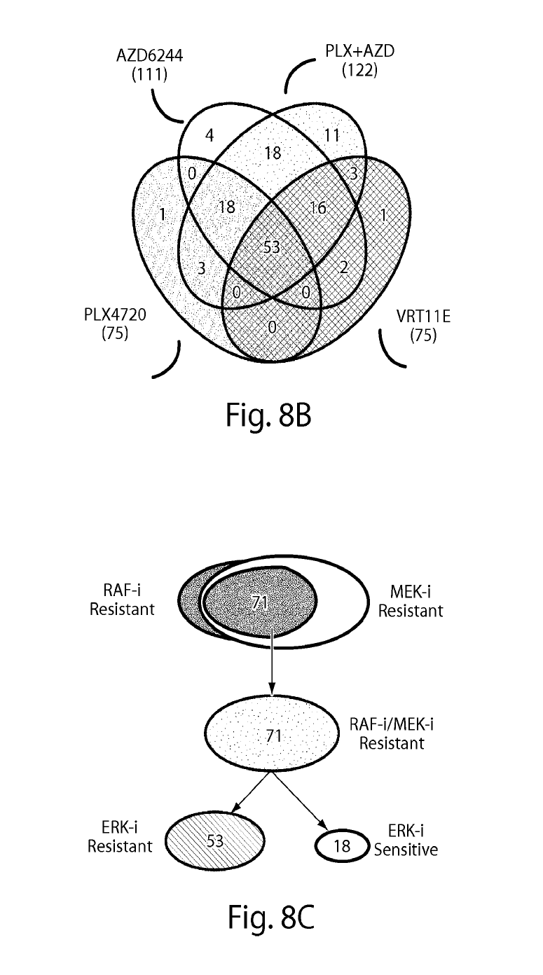

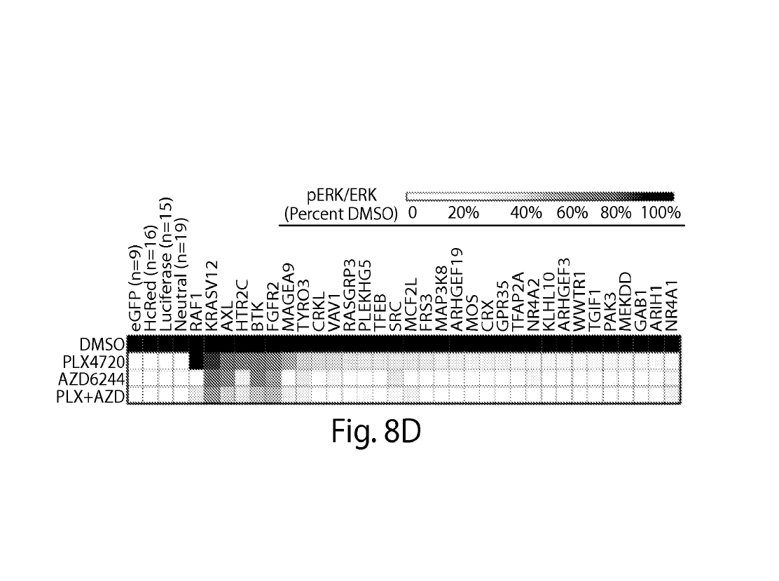

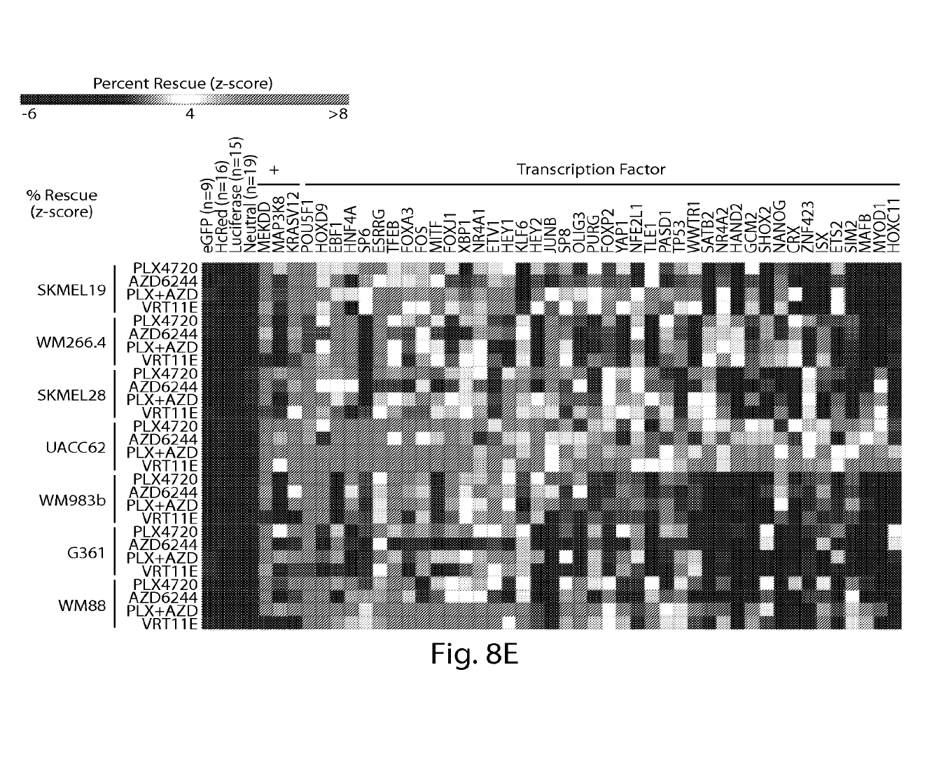

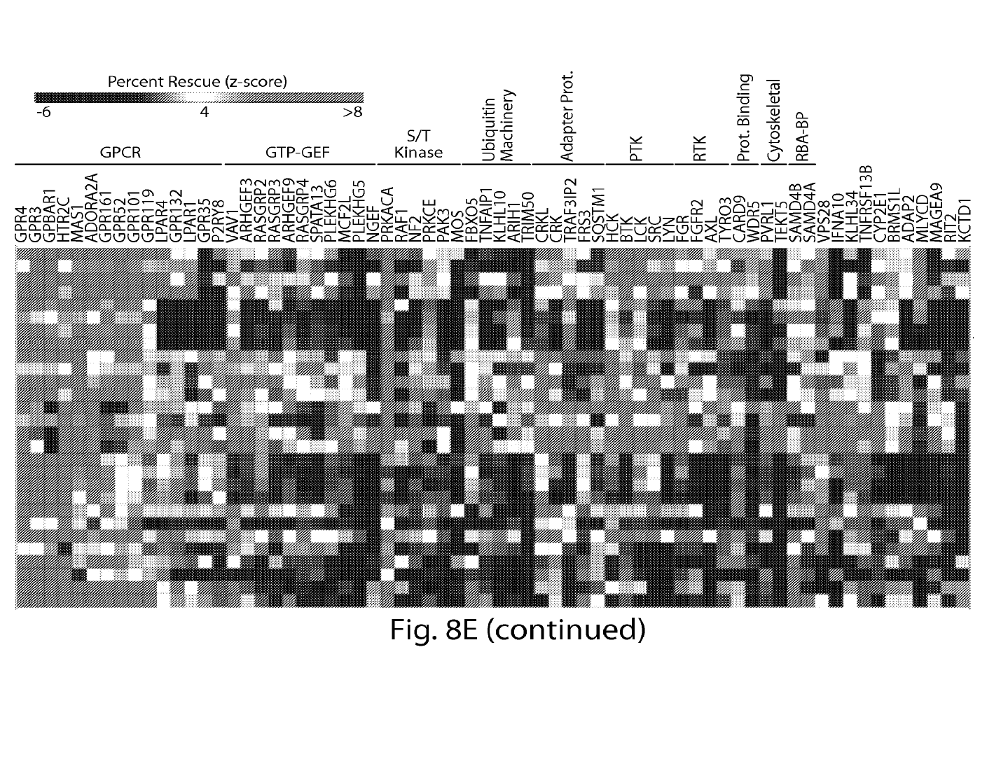

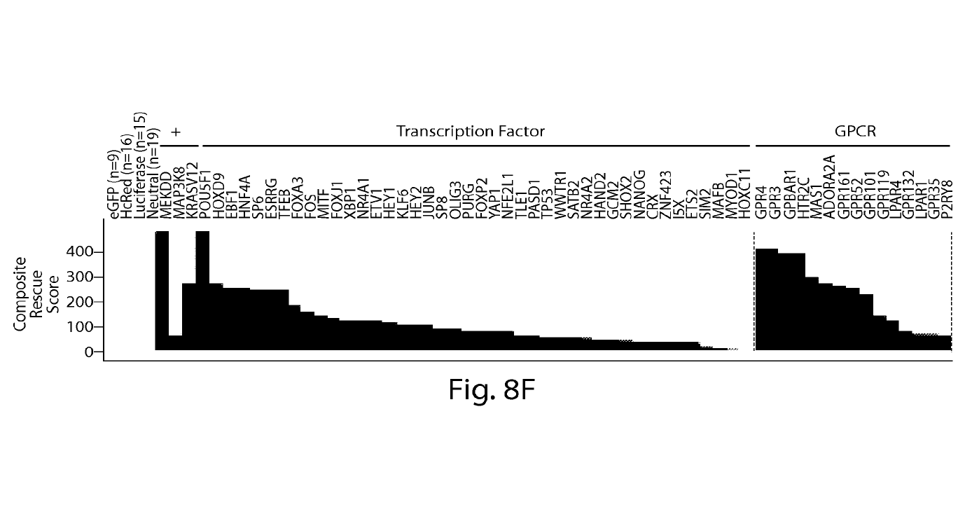

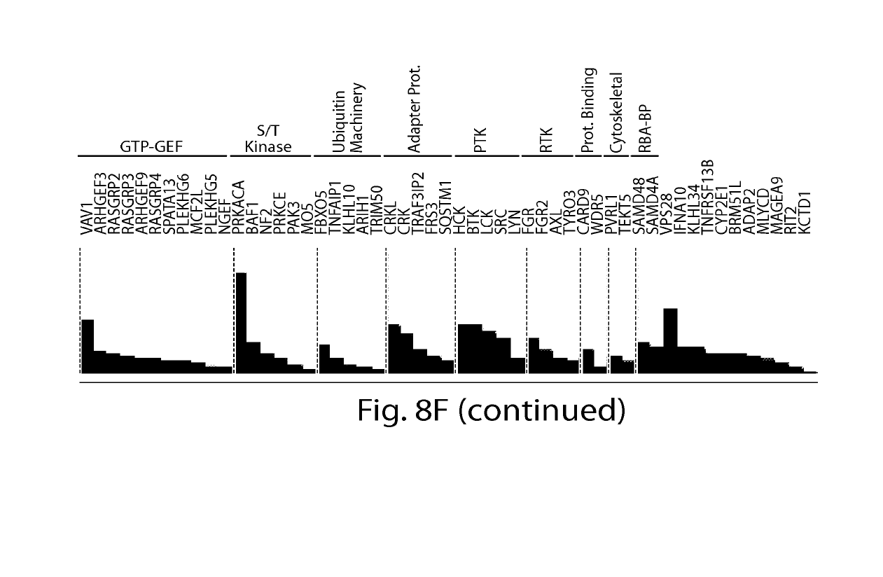

To verify resistance effects, each candidate gene was re-expressed in A375 cells and growth inhibition (GI₅₀) curves were generated for each MAPK pathway inhibitor. A composite drug response metric was determined for each gene (area under the curve; AUC) (FIG. 8a). Concomitant immunoassays confirmed that the drug concentrations employed suppressed MAPK pathway activation. Candidate genes yielding a drug AUC >1.96 standard deviations (p<0.05) from the average of all negative and neutral controls were considered validated hits (FIG. 8a). The percentage of validating genes was 64.2% (RAF-i), 78.4% (MEK-i), 84.5% (RAF/MEK-i) and 75.3% (ERK-i) (FIG. 8a).

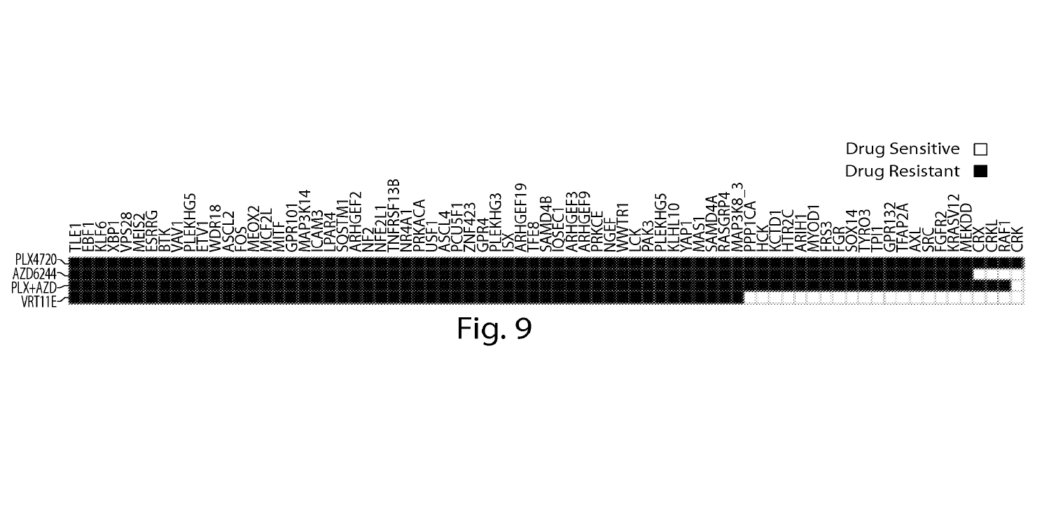

Validated resistance genes frequently conferred resistance to multiple agents (FIG. 8b). For example, 71 of 75 RAF-i resistance genes (94.6%) also imparted resistance to MEK-i (FIG. 8c, FIG. 9). All of the genes that conferred resistance to single agent RAF-i and MEK-i also imparted resistance to combined RAF/MEK-i (FIG. 8c, FIG. 9). Of the 71 genes that induced resistance to RAF-i, MEK-i and combined RAF/MEK-i, only 18 genes (25.4%) retained sensitivity to ERK-i (FIG. 8c, FIG. 9). Thus, the majority of the genes that confer resistance to single agent RAF-i were resistant to both RAF/MEK-i (94.6%) and ERK-i (70.6%) (FIG. 8c and FIG. 9), suggesting that many resistance mechanisms may circumvent the entire RAF/MEK/ERK module.

It was then determined whether the resistance genes could activate the MAPK signaling pathway in the context of RAF-i and/or MEK-i using a pERK assay (FIG. 8d). ERK phosphorylation was induced by MAPKs (MEK1^{DD}/MAP2K1, RAF1 and COT/MAP3K8) or other known pathway activators (e.g., KRAS^{G12V}; FIG. 8d). Aside from a group of tyrosine kinases (AXL, TYRO3, FGR, FGFR2, BTK, SRC), most candidate genes produced only minimal pERK effects (FIG. 8d), consistent with the high degree of ERK-i resistance observed in the validation experiments (FIG. 8a).

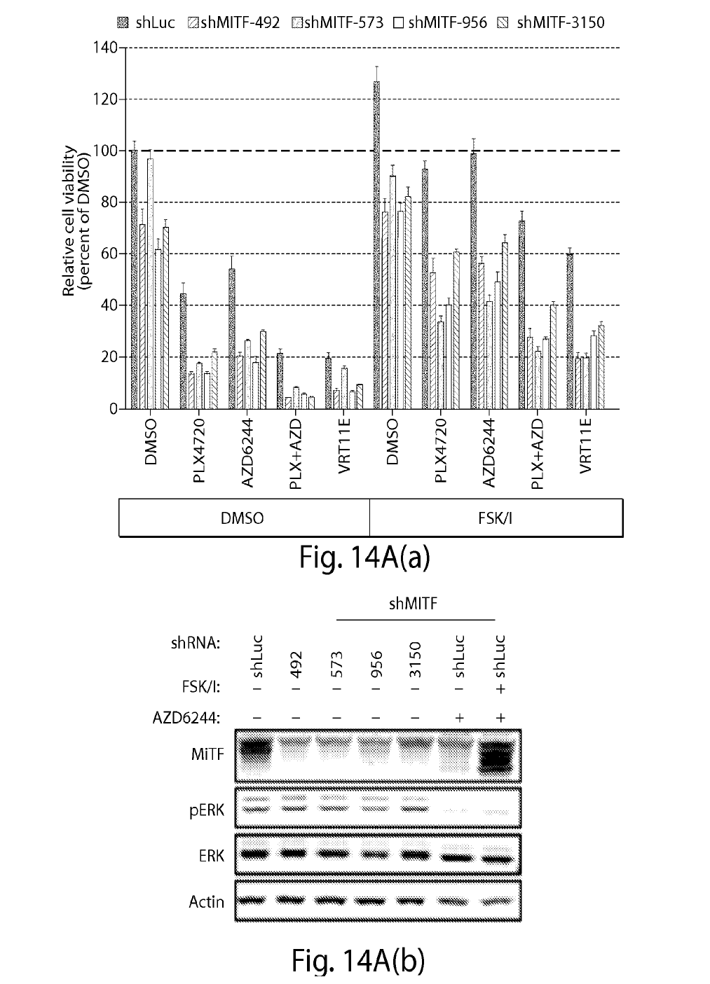

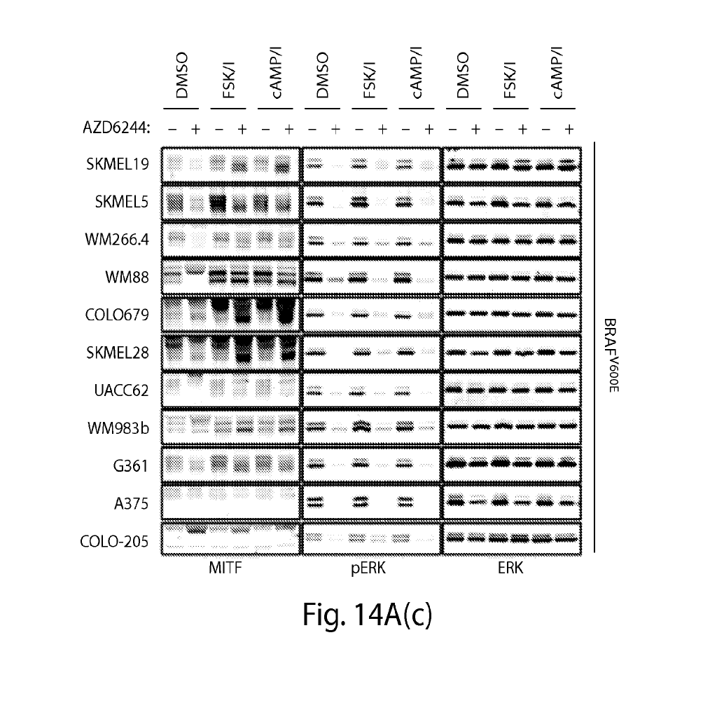

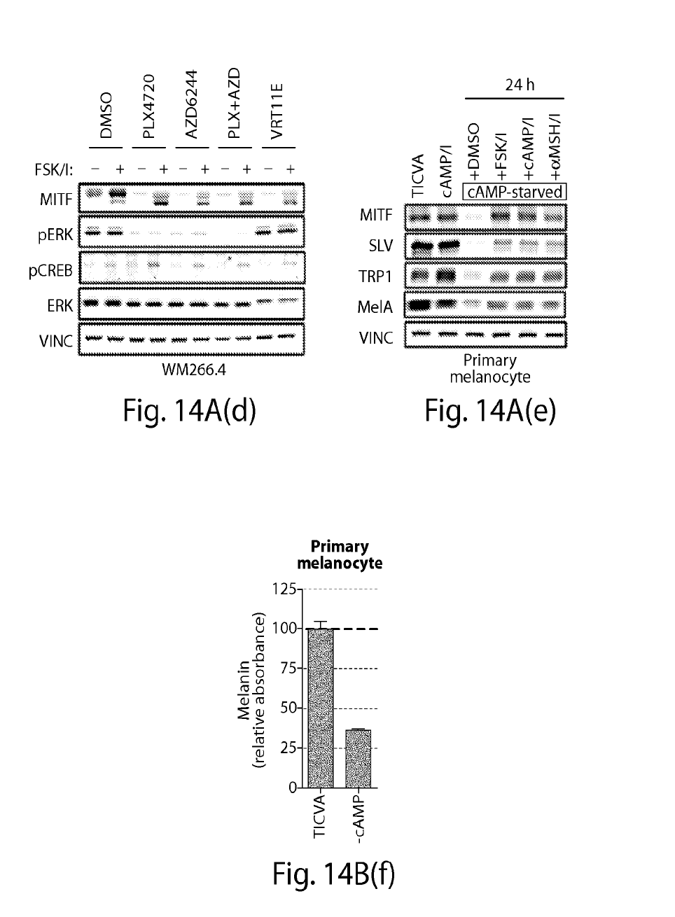

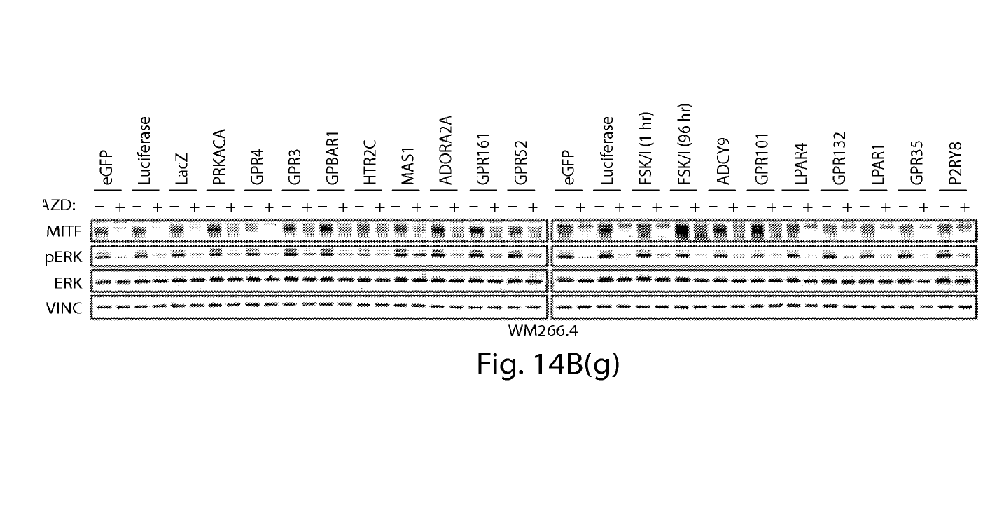

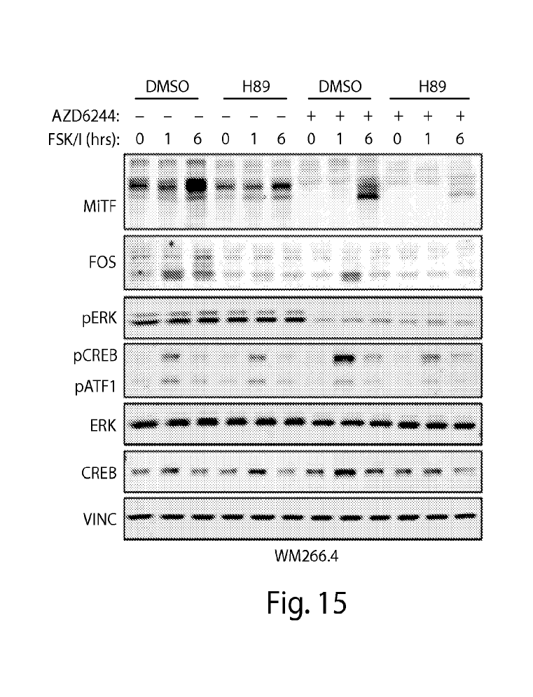

Bona fide resistance genes should modulate drug sensitivity in multiple BRAF^{V600E} melanoma cell lines. Accordingly, the validation of the A375 resistance genes (alongside 59 negative or neutral control genes; FIG. 7A, left panel) was expanded across seven additional drug-sensitive BRAF^{V600E} lines (FIGS. 14A, 14B and 15) that demonstrated comparable infection efficiencies and responses to MAPK pathway inhibitors. Overall, 110 genes (66.7%) conferred resistance to the query inhibitors in at least 2 of 7 additional BRAF^{V600E} melanoma lines (FIG. 8e). Although the magnitude of resistance varied across cell lines, these effects were not attributable to the degree of ectopic expression. Many genes again conferred resistance to all inhibitors/combinations examined, suggesting the existence of multiple ERK-independent resistance effectors (FIG. 8e).

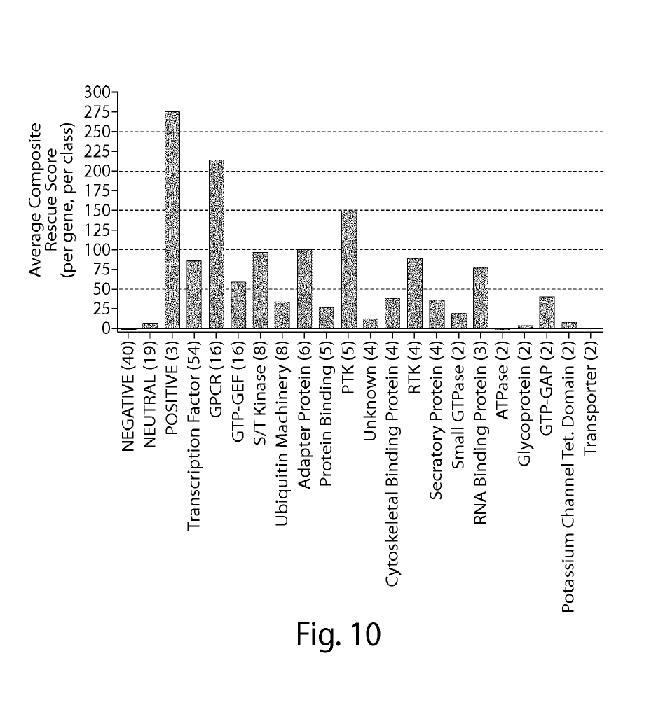

The validated genes were organized into mechanistically related classes and those that exhibited the most extensive validation in the BRAF^{V600E} cell lines were identified. Next, the individual z-score of each gene were summed across all cell lines to create a composite rescue score (ref. 24, FIG. 8f). Calculating the average rescue score within each gene/protein class allowed for ranking of these classes across cell lines (FIG. 10). Based on these criteria, G-protein coupled receptors (GPCRs) emerged as the top ranked protein class (FIG. 10). Each validated GPCR conferred substantial resistance to all MAPK inhibitors tested (FIG. 8e), suggesting an ERK-independent mechanism.

### A Cyclic AMP-Dependent Signaling Network Converges on PKA/CREB to Mediate Resistance to MAPK Pathway Inhibitors

Many GPCRs activate adenyl cyclase (AC)—which catalyzes the conversion of adenosine triphosphate (ATP) to cyclic adenosine monophosphate (cyclic AMP/cAMP) [ref. 25 and 26]. Cyclic AMP binds to protein kinase A (PKA) regulatory subunits, permitting direct phosphorylation of the Cyclic AMP Response Element Binding protein (CREB1, Ser133) and cAMP-dependent Transcription Factor 1 (ATF1, Ser63). CREB1/ATF are transcription factors that regulate the expression of genes whose promoters harbor cyclic AMP response elements (CREs). Consistent with these observations, the AC gene ADCY9 was also identified as a resistance effector (FIG. 7C) and the catalytic subunit of PKAα (PRKACA) had the highest composite rescue score within the Ser/Thr Kinase class (FIG. 8e, 8f). Both genes conferred resistance across all MAPK pathway inhibitors examined (FIG. 8e).

It was hypothesized that a signaling network(s) characterized by GPCR activation and AC/cAMP induction may induce PKA/CREB-driven resistance to MAPK inhibitors in melanoma (FIG. 10a). This predicted network resembles a growth-essential cascade operant in primary melanocytes (the melanoma precursor cell). Primary melanocytes require exogenous cAMP for propagation in vitro and GPCR-mediated cAMP signaling for growth in vivo [ref. 27]. Introducing oncogenic BRAF or NRAS into immortalized melanocytes confers cAMP-independent growth [ref. 28-30]. Conceivably, some MAPK resistance mechanisms might involve aberrant regulation of a known melanocyte lineage dependency.

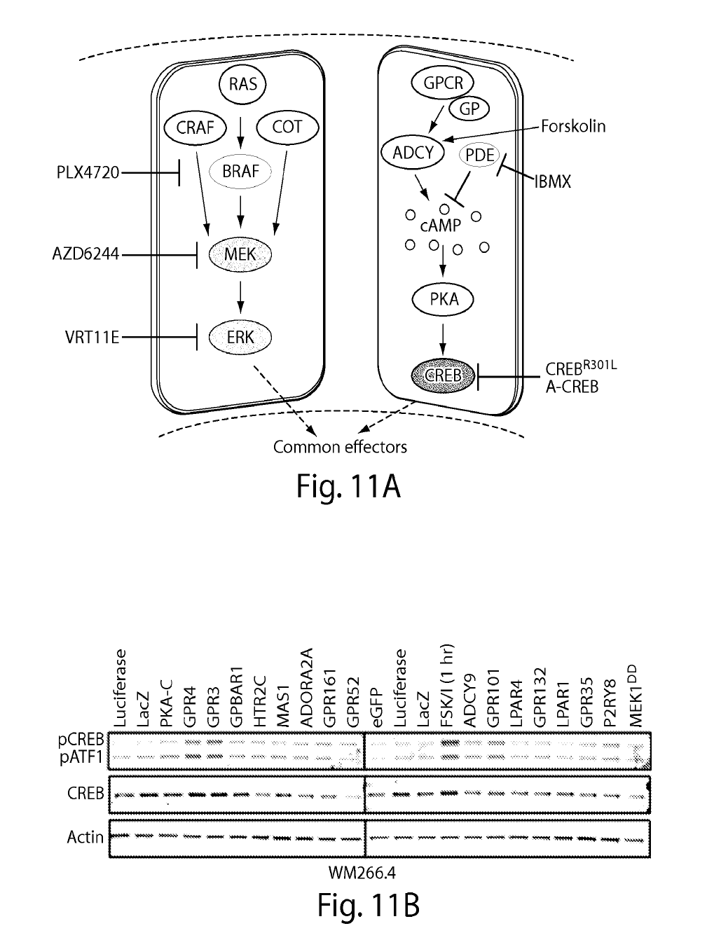

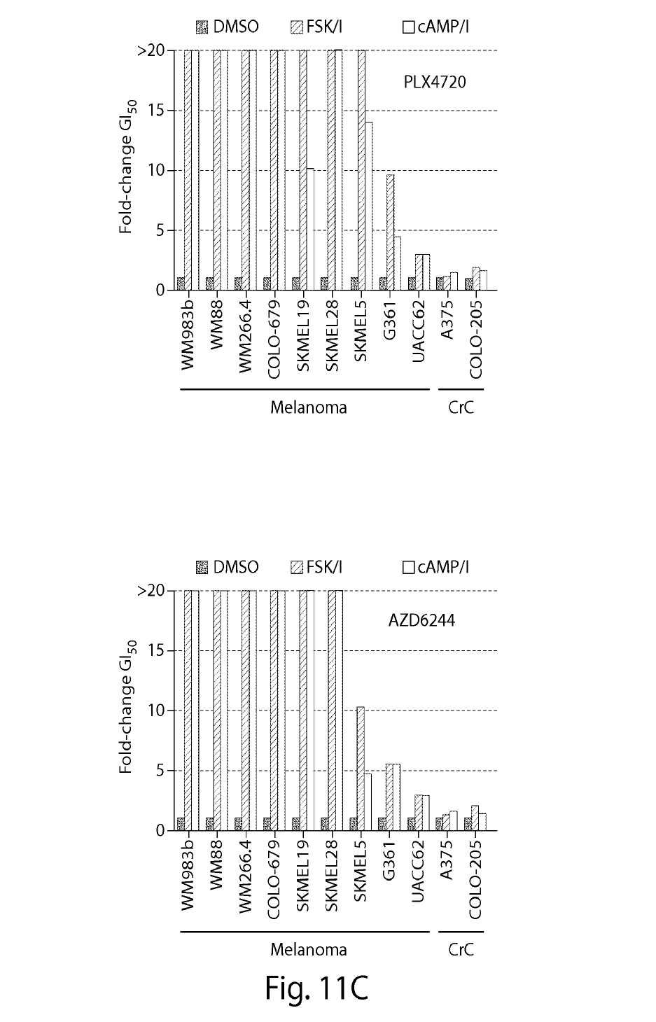

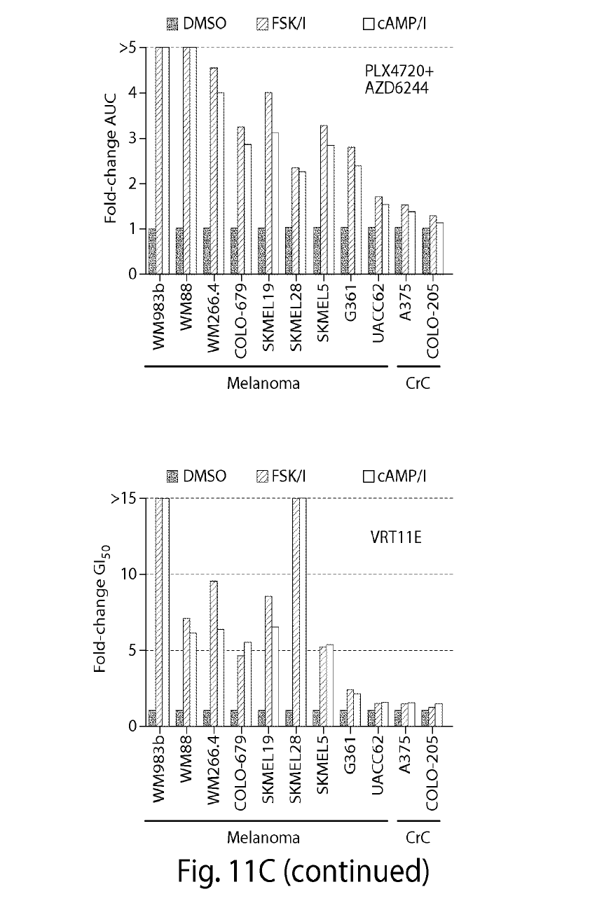

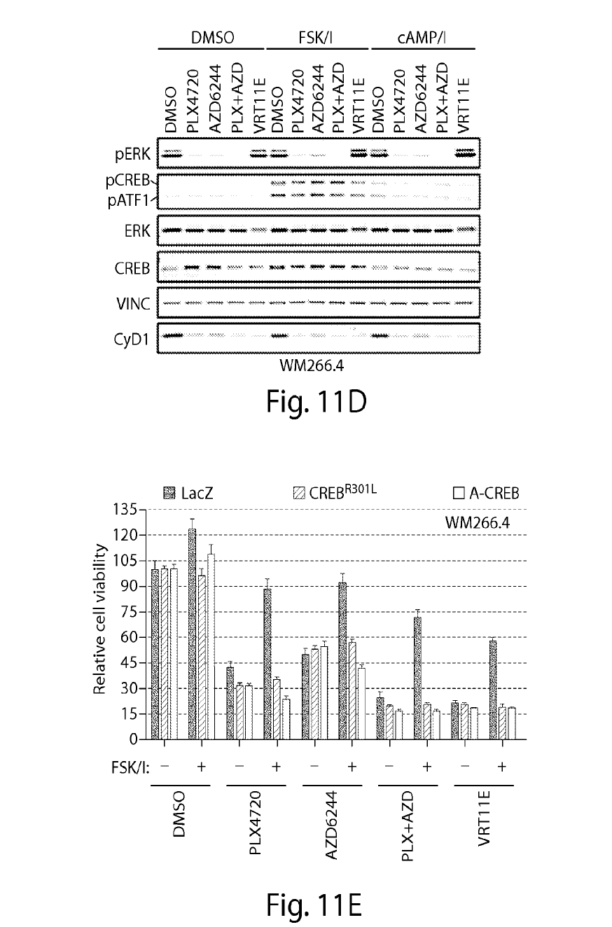

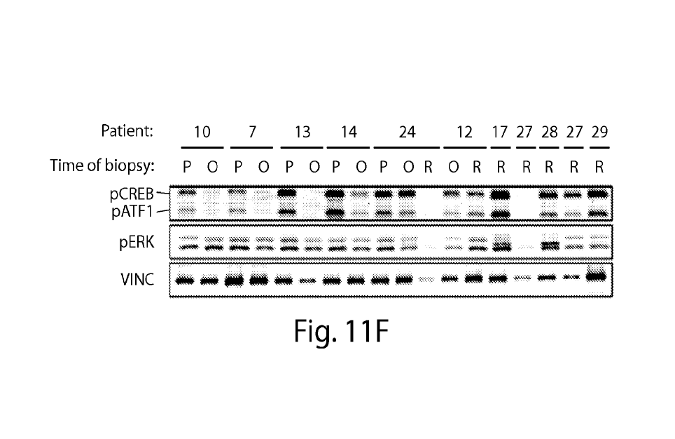

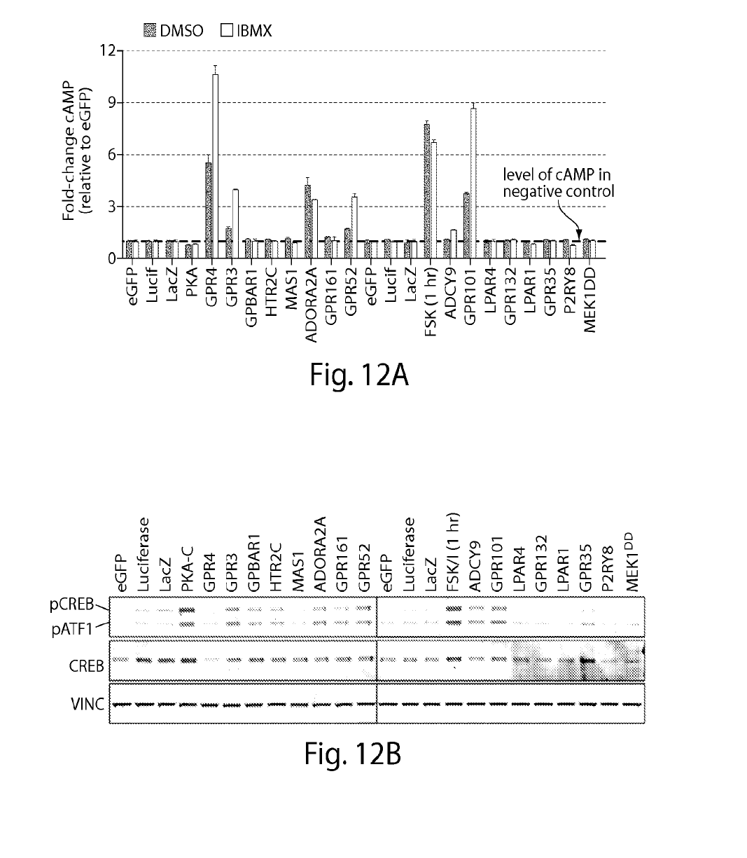

To test this hypothesis, first the effects of resistance-associated GPCRs on CREB phosphorylation when overexpressed in BRAF^{V600E} melanoma cells were analyzed. Despite the transient nature of CREB/ATF1 phosphorylation (FIG. 12), forced GPCR expression produced increases in CREB/ATF1 phosphorylation (FIG. 11 b, FIG. 13a) and some GPCRs produced increases in cAMP formation (FIG. 13b). The GPCRs that failed to induce CREB phosphorylation (LPAR4, GPCR132, LPAR1, GPR35, and P2RY8) also showed a relatively modest resistance phenotype (FIG. 8e, 2f). Thus, CREB phosphorylation correlated with GPCR-mediated resistance in melanoma.

It was next determined if cAMP-mediated signaling was sufficient to confer resistance to MAP kinase pathway inhibitors. Cell growth inhibition assays were performed in multiple BRAF^{V600E} melanoma cell lines using a series of MAPK-pathway inhibitors in the presence of the AC activator forskolin or exogenously-added cAMP. Both forskolin and cAMP conferred resistance to all MAPK-pathway inhibitors queried across the majority of cell lines tested—often by ˜10-fold or higher (FIG. 11c)—without affecting baseline growth. These agents induced CREB phosphorylation with no effect on ERK phosphorylation (FIG. 11d). Forskolin conferred only minimal resistance to a panel of inhibitors that target non-MAPK pathway proteins and were unable to affect MAPK-pathway inhibition in COLO-205, a BRAF^{V600E}-mutant colon carcinoma cell line, suggesting a lineage-specific phenotype (FIG. 11c). A375 was the only melanoma cell line examined whose sensitivity to MAPK pathway inhibition was unaffected by either forskolin or cAMP treatment (FIG. 11c), consistent with the modest validation rate of the GPCR class of candidate resistance genes in A375. These data suggested that GPCR, PKA or AC (ADCY9) overexpression (FIG. 11 b, FIG. 12), stimulation of endogenous adenyl cyclases (forskolin) or treatment with exogenous cAMP (FIG. 11c) may confer CREB-associated and ERK-independent (FIG. 11d) resistance to MAP kinase pathway inhibition (FIG. 8e, 11c).

To confirm that the effects of forskolin/cAMP addition on MAPK inhibitor resistance were CREB dependent, the function of endogenous CREB was interfered with by expressing a dominant-negative CREB allele (CREB^{R301L}) [ref. 31] or the dominant-negative inhibitory protein A-CREB [ref. 32] in the WM266.4 (BRAF^{V600E} melanoma) cell line and measuring their effects on forskolin-induced resistance to MAPK inhibitors (FIG. 11e). The CREB^{R301L} allele remains dimerization-competent, but its DNA-binding activity is impaired [ref. 31], whereas A-CREB binds to endogenous CREB and blocks its ability to bind to DNA [ref. 32]. These reagents both suppressed forskolin-induced resistance to all MAPK-pathway inhibitors tested (FIG. 11e), supporting the hypothesis that cAMP-mediated resistance operates by a CREB dependent mechanism.

These studies identified a signaling network that converges on PKA/CREB to drive resistance to MAPK-pathway inhibitors. It was then determined if this mechanism was evident in biopsies from human tumors that have relapsed following an initial response to MAPK-pathway therapies. In 5 pairs of patient-matched tumor samples, CREB/ATF1 phosphorylation was detectable in biopsies obtained before initiation of MAPK-pathway inhibitor treatment (“P”, FIG. 11f). Following 10-14 days of MAPK-inhibitor therapy (on-treatment, “0”), 4/5 samples showed a marked reduction in CREB/ATF1 phosphorylation, indicative of pathway suppression (FIG. 11f). In 5 of 7 relapsed (R) biopsies, CREB/ATF1 phosphorylation was recovered to levels at or exceeding those observed in pre-treatment samples (FIG. 11f). These data may indicate that CREB/ATF1 activation is a partial determinant of tumor responses to MAPK-inhibitor therapy in a subset of patients. Baseline CREB/ATF1 phosphorylation is low in melanoma cell lines cultured in the absence of extracellular cAMP. However, MAPK pathway signaling impinges on CREB activity through Jun family members (identified here as resistance effectors)—a critical observation that may have foreshadowed in vivo changes in CREB phosphorylation [ref. 33].

Dual Regulation of Transcription Factor Resistance Genes by MAPK and cAMP

It was then hypothesized that a GPCR/cAMP-mediated lineage program might confer resistance to RAF/MEK/ERK inhibition by substituting for oncogenic MAPK signaling in BRAF^{V600E} melanoma cells (FIG. 11a). It was reasoned that a resistance-associated melanocytic linage program may involve CREB-dependent trans-activation of effectors normally under MAPK control in BRAF^{V600E} melanoma and that some of the resistance genes identified herein might represent components of this dually regulated MAP kinase and GPCR/cAMP/CREB transcriptional output (FIG. 8e).

To determine which resistance-associated genes might undergo cAMP/CREB-dependent regulation, promoters of validated resistance genes, the positive and neutral controls were examined for cAMP response elements (CREs). This analysis identified 19 resistance genes—including BRAF—that contained a CRE (no control genes were identified as containing a CRE, FIG. 13a). The representation of CRE-containing genes among our validated resistance genes was significantly enriched over the frequency of CRE-containing genes found within the screening set of ORFS (p=5.0×10⁻⁵⁰). Nine of the CRE-containing genes showed widespread validation (composite resistance score >50; FIG. 13a) and three of these genes—MITF, FOS and NR4A2—encoded transcription factors that are expressed in the melanocyte lineage. MITF encodes the master transcriptional regulator of the melanocyte lineage and is an amplified melanoma oncogenE [ref. 29]. Interestingly, NR4A1 (a NR4A2 homologue) was also a validated resistance gene and has previously been shown to be a PKA/CREB target [ref. 34].

It was then determined if MITF, FOS, NR4A1 or NR4A2 undergo MAP kinase pathway-dependent regulation. Consistent with prior reports [ref. 35 and 36], mRNA levels of each of these genes was suppressed within 6 hours of MEK inhibition, as was expression of DUSP6, an ERK-responsive transcript [ref. 37] (FIG. 13b). MEK inhibition affects MITF mRNA levels only after prolonged MEK inhibition (FIG. 13b). However, MITF phosphorylation was decreased within 1 hour and total MITF was undetectable by 48-96 hours of MEK inhibition (FIG. 13c), consistent with prior studies showing that ERK indirectly regulates MITF mRNA expression [ref. 38 and 39] but directly regulates MITF phosphorylation (the key determinant of its transcriptional activity and stability) [ref. 40 and 41]. These findings suggested that the MAPK pathway may regulate MITF, FOS, NR4A1 and NR4A2 through transcriptional and post-translational mechanisms in BRAF^{V600E} melanoma.

To confirm that MITF, FOS, NR4A1 and NR4A2 were CREB-responsive genes, their expression was assessed following CREB/PKA activation. In the absence of MEK inhibitor, all four genes showed 2- to 20-fold increases in mRNA expression within 1 hour of forskolin treatment. MITF was the only transcript that exhibited sustained expression through 96 hours of forskolin treatment (FIG. 13d). Moreover, only FOS and MITF showed a parallel increase in protein expression (FIG. 13d, 13e). MITF, FOS and NR4A1 all showed a reduction in protein expression following sustained MEK inhibition that could be rescued by forskolin treatment (FIG. 13e). However, MITF was the only gene whose mRNA (FIG. 13d) and protein (FIG. 13e) expression was suppressed by MAPK inhibition and persistently rescued by CREB stimulation. The MITF target genes SILVER and TRP1 showed expression patterns mirroring that of MITF, suggesting that forskolin could regulate MITF function (FIG. 13e). Forskolin-mediated MITF rescue in the presence of MAPK-pathway inhibition was dependent on sustained exposure to forskolin as its removal resulted in rapidly reduced levels of MITF and downstream transcriptional targets. Altogether, these data identified MITF, FOS, NR4A1 and NR4A2, as downstream effectors of both MAPK (FIG. 13b, 13c) and cAMP/PKA/CREB (FIG. 13d, 13e) whose dysregulated expression was sufficient to induce drug resistance (FIG. 8e).

MITF Mediates cAMP-Dependent Resistance to MAPK Pathway Inhibition

Small hairpin RNA (shRNA)-mediated suppression of MITF (FIG. 14A(a), 14A(b)) or expression of a dominant-negative MITF allele (MITF^{R217Δ}) in WM266.4 cells impaired forskolin-mediated resistance to MAPK-pathway inhibitors, suggesting that MITF may be limiting for this phenotype.

To confirm that cAMP-mediated activation of PKA/CREB may provide a generalizable means of rescuing MITF activity a panel of BRAF^{V600E}-mutant melanoma cell lines was treated with a MEK inhibitor alone or in combination with forskolin or cAMP (FIG. 13f). Forskolin and cAMP reversed MEK-inhibitor mediated suppression of MITF protein levels in all cell lines that exhibited robust basal MITFm expression (FIG. 14A(c)). Notably, A375 were the only melanoma cell line tested that lacked MITF expression, which may explain their modest response to forskolin/cAMP (FIG. 11c, 14A(c)). Reductions in MITF protein and rescue by forskolin were observed following treatment with RAF, RAF/MEK or ERK inhibitors (FIG. 14A(d)). Analogous results were observed in primary melanocytes, where removal of cAMP/IBMX from the culture media resulted in markedly decreased MITF protein expression, reduced expression of the MITF target genes SLV, TRP1 and Melan-A (FIG. 14A(e)) and a decrease in melanin content (FIG. 14B(f)). Forskolin-mediated rescue of MITF protein expression was largely abrogated by treatment with a small molecule PKA inhibitor (H89) (FIG. 15), consistent with a dependence on PKA/CREB for cAMP-dependent control of MITF expression.

To determine if expression of the GPCRs identified in the functional screens described herein could regulate MITF levels in melanoma cells, ORFS corresponding to the relevant GPCRs, PKA (PRKACA) or AC (ADCY9) were expressed in WM266.4 cells and MITF expression was examined in the presence or absence of pharmacologic MAPK inhibition. Expression of PKAα, ADCY9 or a subset of the GPCRs enabled sustained MITF expression, even in the setting of MEK inhibition (FIG. 14B(g)), thereby confirming that dysregulated GPCR or PKA/AC activity regulates MITF expression in BRAF^{V600E} melanoma cells treated with MAPK pathway inhibitors.

Combined MAPK/HDAC Inhibition Overcomes cAMP-Dependent Resistance

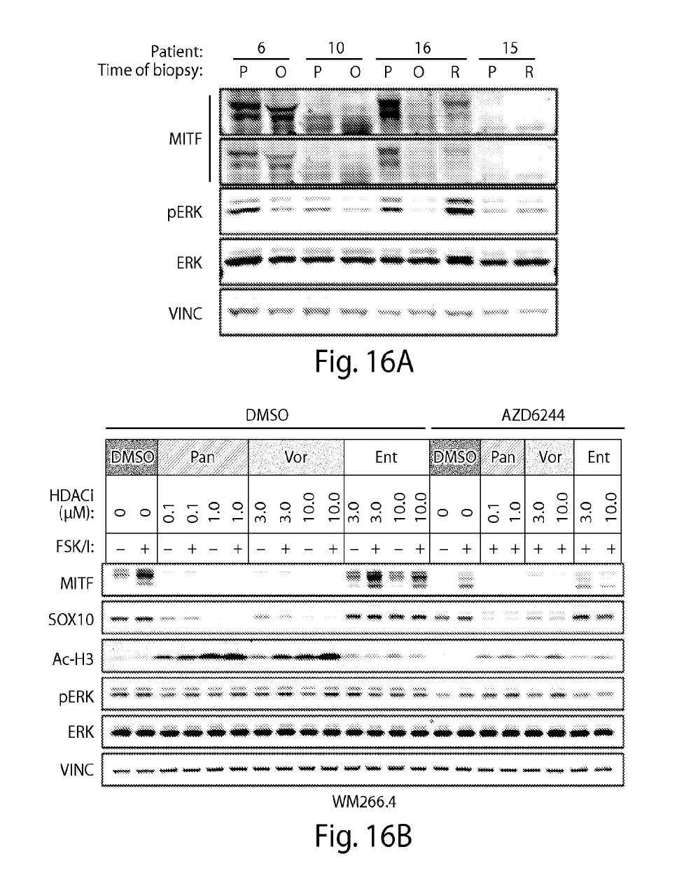

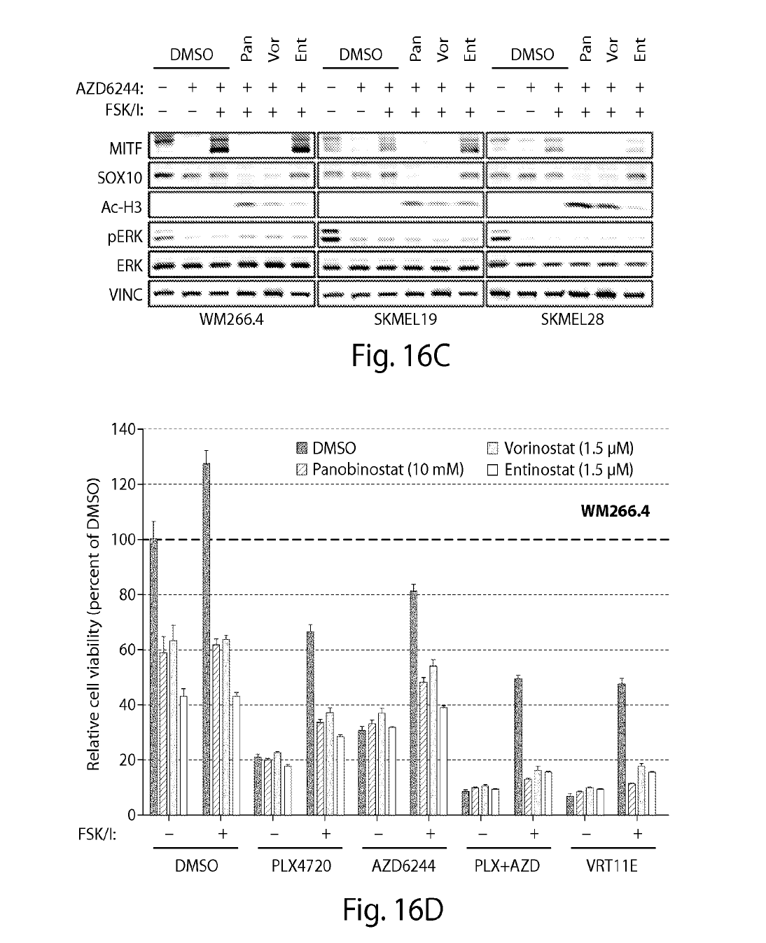

Emerging treatment modalities for BRAF^{V600E}-melanomas have focused on combinatorial targeting of RAF/MEK/ERK kinases [ref. 3]. However, the data presented here predict that aberrant signaling from melanocyte lineage pathways or other bypass mechanisms may converge on shared downstream transcriptional effectors in general—and MITF in particular—to drive MEK/ERK-independent therapeutic resistance. To test the possibility that aberrant MITF re-expression may contribute to drug resistance in human tumors, MITF and ERK phosphorylation levels were examined in lysates from melanoma biopsies. Two of 4 samples showed detectable MITF expression in the pre-treatment (P) biopsy (FIG. 16a). Following 10-14 days of MAPK-pathway inhibitor treatment, MITF expression was sustained in one patient (pt. 6, “O”), but undetectable in the other (pt. 16, “O”) despite a reduction in pERK levels in both patients (FIG. 16a). In the one patient-matched trio tested (pre, on, relapse), MITF was detectable in the context of relapse (FIG. 16a), potentially owning to re-activated ERK phosphorylation (FIG. 16a). These data suggest that in a subset of patients, MITF may represent a viable drug target when combined with MAPK-pathway inhibitors. Accordingly, combined (shRNA-mediated) impairment of MITF cooperates with MAPK [ref. 45] pathway inhibition in vitro (FIG. 14A(a)).

While direct therapeutic targeting of oncogenic transcription factors remains challenging, indirect pharmacological inhibition of MITF expression by histone deacetylase inhibitors (HDACi) has been reported [ref. 46]. Thus, it was hypothesized that adding an HDAC inhibitor to combined RAF/MEK inhibition might prevent resistance-associated rescue of MITF protein levels and enable suppression of BRAF^{V600E} melanoma cell growth. To test this hypothesis, WM266.4 (BRAF^{V600E}) melanoma cells were exposed to three HDAC inhibitors that have been examined clinically, including Panobinostat/LBH589 and Vorinostat/SAHA and the less potent Entinostat/MS275. Both Panobinostat and Vorinostat produced increases in acetylated histone H3 and a reduction in SOX10 and MITF expression independent of ERK phosphorylation (FIG. 16b). In the presence of a MEK inhibitor, MITF expression was reduced (FIG. 16b) and concomitant exposure to HDAC inhibitors suppressed MITF protein following forskolin treatment. Moreover, HDACi treatment impaired MITF re-expression in a number of BRAF^{V600E}-mutant melanoma cell lines (FIG. 16b, 16c), suggesting that the effects of HDAC inhibitors are dominant to GPCR/cAMP/CREB signaling effects.

Next, the consequences of HDAC-inhibitor mediated reduction of MITF expression on the growth of BRAF^{V600E} melanoma cells rendered resistant to the effects of RAF/MEK/ERK inhibitors was tested. Indeed, exposure of forskolin-treated WM266.4 cells to sub-lethal doses of Panobinostat, Vorinostat or Entinostat restored sensitivity to MAPK-pathway inhibitors to levels approaching parental cells (FIG. 16d). Accordingly, the addition of HDAC inhibitors to combined RAF/MEK inhibitor or single RAF, MEK, ERK inhibitors offers a novel clinical strategy to achieve more durable control of BRAF^{V600E} melanoma.

### REFERENCES

1. Solit, D. B. et al. BRAF mutation predicts sensitivity to MEK inhibition. Nature 439, 358-362, doi:nature04304 [pii]10.1038/nature04304 (2006).
2. Chapman, P. B. et al. Improved survival with vemurafenib in melanoma with BRAF V600E mutation. N Engl J Med 364, 2507-2516, doi:10.1056/NEJMoa1103782 (2011).
3. Flaherty, K. T. et al. Combined BRAF and MEK inhibition in melanoma with BRAF V600 mutations. N Engl J Med 367, 1694-1703, doi:10.1056/NEJMoa1210093 (2012).
4. Flaherty, K. T. et al. Improved survival with MEK inhibition in BRAF-mutated melanoma. N Engl J Med 367, 107-114, doi:10.1056/NEJMoa1203421 (2012).
5. Kaplan, F. M. et al. SHOC2 and CRAF mediate ERK1/2 reactivation in mutant NRAS-mediated resistance to RAF inhibitor. J Biol Chem 287, 41797-41807, doi:10.1074/jbc.M112.390906 (2012).
6. Nazarian, R. et al. Melanomas acquire resistance to B-RAF(V600E) inhibition by RTK or N-RAS upregulation. Nature 468, 973-977, doi:10.1038/nature09626 (2010).
7. Poulikakos, P. I. et al. RAF inhibitor resistance is mediated by dimerization of aberrantly spliced BRAF(V600E). Nature 480, 387-390, doi:nature10662 [pii]10.1038/nature10662 (2011).
8. Shi, H. et al. Melanoma whole-exome sequencing identifies (V600E)B-RAF amplification-mediated acquired B-RAF inhibitor resistance. Nat Commun 3, 724, doi:ncomms1727 [pii]10.1038/ncomms1727 (2012).
9. Little, A. S. et al. Amplification of the driving oncogene, KRAS or BRAF, underpins acquired resistance to MEK1/2 inhibitors in colorectal cancer cells. Sci Signal 4, ra17, doi:4/166/ra17 [pii]10.1126/scisignal.2001752 (2011).
10. Corcoran, R. B. et al. BRAF gene amplification can promote acquired resistance to MEK inhibitors in cancer cells harboring the BRAF V600E mutation. Sci Signal 3, ra84, doi:3/149/ra84 [pii]10.1126/scisignal.2001148 (2010).
11. Johannessen, C. M. et al. COT drives resistance to RAF inhibition through MAP kinase pathway reactivation. Nature 468, 968-972, doi:nature09627 [pii]10.1038/nature09627 (2010).
12. Straussman, R. et al. Tumour micro-environment elicits innate resistance to RAF inhibitors through HGF secretion. Nature 487, 500-504, doi:10.1038/nature11183 (2012).
13. Villanueva, J. et al. Acquired resistance to BRAF inhibitors mediated by a RAF kinase switch in melanoma can be overcome by cotargeting MEK and IGF-1 R/PI3K. Cancer Cell 18, 683-695, doi:10.1016/j.ccr.2010.11.023 (2010).
14. Emery, C. M. et al. MEK1 mutations confer resistance to MEK and B-RAF inhibition. Proc Natl Acad Sci USA 106, 20411-20416, doi:10.1073/pnas.0905833106 (2009).
15. Wagle, N. et al. Dissecting therapeutic resistance to RAF inhibition in melanoma by tumor genomic profiling. J Clin Oncol 29, 3085-3096, doi:10.1200/JCO.2010.33.2312 (2011).
16. Tsao, H., Chin, L., Garraway, L. A. & Fisher, D. E. Melanoma: from mutations to medicine. Genes Dev 26, 1131-1155, doi:10.1101/gad.191999.112 (2012).
17. Yang, X. et al. A public genome-scale lentiviral expression library of human ORFS. Nat Methods 8, 659-661, doi:nmeth.1638 [pii]10.1038/nmeth.1638 (2011).
18. Wood, K. C. et al. MicroSCALE screening reveals genetic modifiers of therapeutic response in melanoma. Sci Signal 5, rs4, doi:5/224/rs4 [pii]10.1126/scisignal.2002612 (2012).
19. Aronov, A. M. et al. Structure-guided design of potent and selective pyrimidylpyrrole inhibitors of extracellular signal-regulated kinase (ERK) using conformational control. J Med Chem 52, 6362-6368, doi:10.1021/jm900630q (2009).
20. Crews, C. M., Alessandrini, A. & Erikson, R. L. The primary structure of MEK, a protein kinase that phosphorylates the ERK gene product. Science 258, 478-480 (1992).
21. Kyriakis, J. M. et al. Raf-1 activates MAP kinase-kinase. Nature 358, 417-421, doi:10.1038/358417a0 (1992).
22. Patriotis, C., Makris, A., Chernoff, J. & Tsichlis, P. N. Tpl-2 acts in concert with Ras and Raf-1 to activate mitogen-activated protein kinase. Proc Natl Acad Sci USA 91, 9755-9759 (1994).
23. Pham, C. D., Arlinghaus, R. B., Zheng, C. F., Guan, K. L. & Singh, B. Characterization of MEK1 phosphorylation by the v-Mos protein. Oncogene 10, 1683-1688 (1995).
24. Kalkers, N. F. et al. MS functional composite: relation to disease phenotype and disability strata. Neurology 54, 1233-1239 (2000).
25. Lonze, B. E. & Ginty, D. D. Function and regulation of CREB family transcription factors in the nervous system. Neuron 35, 605-623 (2002).
26. Xiao, R. P. Beta-adrenergic signaling in the heart: dual coupling of the beta2-adrenergic receptor to G(s) and G(i) proteins. Science's STKE: signal transduction knowledge environment 2001, re15, doi:10.1126/stke.2001.104.re15 (2001).
27. Hayward, N. K. Genetics of melanoma predisposition. Oncogene 22, 3053-3062, doi:10.1038/sj.onc.12064451206445 [pii] (2003).
28. Dumaz, N. et al. In melanoma, RAS mutations are accompanied by switching signaling from BRAF to CRAF and disrupted cyclic AMP signaling. Cancer Res 66, 9483-9491, doi:66/19/9483 [pii]10.1158/0008-5472.CAN-05-4227 (2006).
29. Garraway, L. A. et al. Integrative genomic analyses identify MITF as a lineage survival oncogene amplified in malignant melanoma. Nature 436, 117-122, doi:nature03664 [pii]10.1038/nature03664 (2005).
30. Jane-Valbuena, J. et al. An oncogenic role for ETV1 in melanoma. Cancer Res 70, 2075-2084, doi:0008-5472.CAN-09-3092 [pii]10.1158/0008-5472.CAN-09-3092 (2010).
31. Walton, K. M., Rehfuss, R. P., Chrivia, J. C., Lochner, J. E. & Goodman, R. H. A dominant repressor of cyclic adenosine 3′,5′-monophosphate (cAMP)-regulated enhancer-binding protein activity inhibits the cAMP-mediated induction of the somatostatin promoter in vivo. Molecular endocrinology 6, 647-655 (1992).
32. Ahn, S. et al. A dominant-negative inhibitor of CREB reveals that it is a general mediator of stimulus-dependent transcription of c-fos. Mol Cell Biol 18, 967-977 (1998).
33. Lopez-Bergami, P. et al. Rewired ERK-JNK signaling pathways in melanoma. Cancer Cell 11, 447-460, doi:10.1016/j.ccr.2007.03.009 (2007).
34. Maruoka, H. et al. Dibutyryl-cAMP up-regulates nur77 expression via histone modification during neurite outgrowth in PC12 cells. Journal of biochemistry 148, 93-101, doi:10.1093/jb/mvq036 (2010).
35. Eferl, R. & Wagner, E. F. AP-1: a double-edged sword in tumorigenesis. Nat Rev Cancer 3, 859-868, doi:10.1038/nrc1209nrc1209 [pii] (2003).
36. Smith, A. G., Lim, W., Pearen, M., Muscat, G. E. & Sturm, R. A. Regulation of NR4A nuclear receptor expression by oncogenic BRAF in melanoma cells. Pigment Cell Melanoma Res 24, 551-563, doi:10.1111/j.1755-148X.2011.00843.x (2011).
37. Joseph, E. W. et al. The RAF inhibitor PLX4032 inhibits ERK signaling and tumor cell proliferation in a V600E BRAF-selective manner. Proc Natl Acad Sci USA 107, 14903-14908, doi:1008990107 [pii]10.1073/pnas.1008990107 (2010).
38. Kono, M. et al. Role of the mitogen-activated protein kinase signaling pathway in the regulation of human melanocytic antigen expression. Mol Cancer Res 4, 779-792, doi:4/10/779 [pii]10.1158/1541-7786.MCR-06-0077 (2006).
39. Wellbrock, C. et al. Oncogenic BRAF regulates melanoma proliferation through the lineage specific factor MITF. PLoS One 3, e2734, doi:10.1371/journal.pone.0002734 (2008).
40. Hemesath, T. J., Price, E. R., Takemoto, C., Badalian, T. & Fisher, D. E. MAP kinase links the transcription factor Microphthalmia to c-Kit signalling in melanocytes. Nature 391, 298-301, doi:10.1038/34681 (1998).
41. Wu, M. et al. c-Kit triggers dual phosphorylations, which couple activation and degradation of the essential melanocyte factor Mi. Genes Dev 14, 301-312 (2000).
42. Hodgkinson, C. A. et al. Mutations at the mouse microphthalmia locus are associated with defects in a gene encoding a novel basic-helix-loop-helix-zipper protein. Cell 74, 395-404, doi:0092-8674(93)90429-T [pii] (1993).
43. Davis, I. J. et al. Oncogenic MITF dysregulation in clear cell sarcoma: defining the MiT family of human cancers. Cancer Cell 9, 473-484, doi:S1535-6108(06)00144-9 [pii]10.1016/j.ccr.2006.04.021 (2006).
44. Wang, W. L. et al. Detection and characterization of EWSR1/ATF1 and EWSR1/CREB1 chimeric transcripts in clear cell sarcoma (melanoma of soft parts). Mod Pathol 22, 1201-1209, doi:modpathol200985 [pii]10.1038/modpathol.2009.85 (2009).
45. Kido, K. et al. Simultaneous suppression of MITF and BRAF V600E enhanced inhibition of melanoma cell proliferation. Cancer science 100, 1863-1869, doi:10.1111/j.1349-7006.2009.01266.x (2009).
46. Yokoyama, S. et al. Pharmacologic suppression of MITF expression via HDAC inhibitors in the melanocyte lineage. Pigment Cell Melanoma Res 21, 457-463, doi:PCR480 [pii]10.1111/j.1755-148X.2008.00480.x (2008).
47. Price, E. R. et al. alpha-Melanocyte-stimulating hormone signaling regulates expression of microphthalmia, a gene deficient in Waardenburg syndrome. J Biol Chem 273, 33042-33047 (1998).
48. Huber, W. E. et al. A tissue-restricted cAMP transcriptional response: SOX10 modulates alpha-melanocyte-stimulating hormone-triggered expression of microphthalmia-associated transcription factor in melanocytes. J Biol Chem 278, 45224-45230, doi:10.1074/jbc.M309036200M309036200 [pii] (2003).
49. Oka, M., Ichihashi, M. & Chakraborty, A. K. Enhanced expression of protein kinase C subspecies in melanogenic compartments in B16 melanoma cells by UVB or MSH. The Journal of investigative dermatology 106, 377-378 (1996).

All references recited herein are incorporated by reference herein in their entirety. The definitions and disclosures provided herein govern and supersede all others incorporated by reference. Although the invention herein has been described in connection with preferred embodiments thereof, it will be appreciated by those skilled in the art that additions, modifications, substitutions, and deletions not specifically described may be made without departing from the spirit and scope of the invention as defined in the appended claims. It is therefore intended that the foregoing detailed description be regarded as illustrative rather than limiting, and that it be understood that it is the following claims, including all equivalents, that are intended to define the spirit and scope of this invention.

What is claimed is:

1. A method comprising:
(a) assaying, in cancer cells from a subject having cancer, a gene copy number, mRNA or protein level, or activity level of a marker selected from:
(i) GPCRs that activate production of cyclic AMP, and
(ii) GPCR pathway components selected from the group consisting of PKA, FOS, NR4A1, NR4A2, MITF, and a PKA-activated transcription factor that activates FOS, NR4A1, NR4A2, and MITF,
(b) comparing the gene copy number, mRNA or protein level, or activity level of the marker in the cancer cells with a gene copy number, mRNA or protein level, or activity level of the marker in normal cells, and
(c) identifying a subject having cancer cells with increased gene copy number, mRNA or protein level, or activity level of the marker relative to normal cells as a subject (i) who is at risk of developing resistance to a MAPK pathway inhibitor, (ii) who is likely to benefit from treatment with an HDAC inhibitor, (iii) who is likely to benefit from treatment with a combination therapy comprising an HDAC inhibitor, and/or (iv) who is likely to benefit from treatment with a combination therapy comprising a MAPK pathway inhibitor and an HDAC inhibitor.

2. A method comprising:
(a) assaying, in cancer cells from a subject having cancer, a gene copy number, mRNA or protein level, or activity level of a marker selected from:
(i) GEFs selected from the group consisting of ARHGEF2, ARHGEF3, ARHGEF9, ARHGEF19, MCF2L, NGEF, VAV1, PLEKHG3, PLEKHG5, PLEKHG6, IQSEC1, TBC1D3G, SPATA13, RASGRP2, RASGRP3, and RASGRP4,
(ii) GPCRs that activate production of cyclic AMP,
(iii) GPCR pathway components selected from the group consisting of PKA, FOS, NR4A1, NR4A2, MITF, and a PKA-activated transcription factor that activates FOS, NR4A1, NR4A2, and MITF,
(iv) transcription factors selected from the group consisting of POU51, HOXD9, EBF1, HNF4A, SP6, ESRRG, TFEB, FOXA3, FOS, MITF, FOXJ1, XBP1, NR4A1, ETV1, HEY1, KLF6, HEY2, JUNB, SP8, OLIG3, PURG, FOXP2, YAP1, NFE2L1, TLE1, PASD1, TP53, WWTR1, SATB2, NR4A2, HAND2, GCM2, SHOX2, NANOG, CRX, ZNF423, ISX, ETS2, SIM2, MAFB, MYOD1, and HOXC11,
(v) serine/threonine kinases selected from the group consisting of PRKACA, RAF1, NF2, PRKCE, PAK3, and MOS,
(vi) ubiquitin machinery proteins selected from the group consisting of FBX05, TNFAIP1, KLHL10, ARIH1, and TRIM50,
(vii) adaptor proteins selected from the group consisting of CRKL, CRK, TRAF3IP1, FRS3, AND SQSTM1,
(viii) protein tyrosine kinases selected from the group consisting of HCK, BTK, LCK, SRC, and LYNp,
(ix) receptor tyrosine kinases selected from the group consisting of FGR, FGFR2, AXL, and TYRO3,
(x) protein binding proteins selected from the group consisting of CARD9 and WDR5,
(xi) cytoskeletal proteins selected from the group consisting of PVRL1 and TEKT5,
(xii) RNA binding proteins selected from the group consisting of SAMD4B and SAMD4A, and
(xiii) VPS28, IFNA10, KLHL34, TNFRSF13B, CYP2E1, BRMS1L, ADAP2, MLYCD, MAGEA9, RIT2, and KCTD1,
(b) comparing the gene copy number, mRNA or protein level, or activity level of the marker in the cancer cells with a gene copy number, mRNA or protein level, or activity level of the marker in normal cells, and
(c) identifying a subject having cancer cells with increased gene copy number, mRNA or protein level, or activity level of the marker relative to normal cells as a subject who is at risk of developing resistance to a MAPK pathway inhibitor.

3. The method of claim 1 or 2, wherein the GPCRs that activate production of cyclic AMP are selected from the group consisting of GPR4, GPR3, GPBAR1, HTR2C, MAS1, ADORA2A, GPR161, GPR52, GPR101, and GPR119.

4. The method of any one of claims 1-3, wherein the PKA-activated transcription factor that activates FOS, NR4A1, NR4A2, and MITF is selected from the group consisting of CREB1, ATF4, ATF1, CREB3, CREB5, CREB3L1, CREB3L2, CREB3L3, and CREB3L4.

5. The method of any one of claims 1-4, wherein the cancer is selected from the group consisting of melanoma, breast cancer, colorectal cancer, glioma, lung cancer, ovarian cancer, sarcoma and thyroid cancer.

6. The method of claim 5, wherein the cancer is melanoma.

7. The method of any one of claims 1-6, wherein the cancer cells comprise a mutation in B-RAF.

8. The method of claim 7, wherein the cancer cells comprise a B-RAF^{V600E} mutation.

9. The method of any one of claims 1-8, wherein the subject has received a therapy comprising a MAPK pathway inhibitor.

10. The method of claim 9, wherein the subject has manifest resistance to the MAPK pathway inhibitor.

11. The method of any one of claims 1-10, wherein the MAPK pathway inhibitor is a RAF inhibitor.

12. The method of any one of claims 1-11, wherein the MAPK pathway inhibitor is a pan-RAF inhibitor.

13. The method of any one of claims 1-11, wherein the MAPK pathway inhibitor is a selective RAF inhibitor.

14. The method of claim 13, wherein the RAF inhibitor is selected from the group consisting of RAF265, sorafenib, dabrafenib (GSK2118436), SB590885, PLX 4720, PLX4032, GDC-0879 and ZM 336372.

15. The method of any one of claims 1-10, wherein the MAPK pathway inhibitor is a MEK inhibitor.

16. The method of claim 15, wherein the MEK inhibitor is selected from the group consisting of CI-1040/PD184352, AZD6244, PD318088, PD98059, PD334581, RDEA119, 6-Methoxy-7-(3-morpholin-4-yl-propoxy)-4-(4-phenoxy-phenylamino)-quinoline-3-carbonitrile and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, trametinib (GSK1120212), and ARRY-438162.

17. The method of any one of claims 1-10, wherein the MAPK pathway inhibitor is two MAPK pathway inhibitors, and wherein one of a first of the two MAPK inhibitors is a RAF inhibitor and a second of the two MAPK inhibitors is a MEK inhibitor.

18. The method of any one of claims 1-10, wherein the MAPK pathway inhibitor is an ERK inhibitor.

19. The method of claim 18, wherein the ERK inhibitor is selected from the group consisting of VTX11e, AEZS-131, PD98059, FR180204, and FR148083.

20. The method of claim 1, wherein the HDAC inhibitor is selected from the group consisting of Vorinostat, CI-994, Entinostat, BML-210, M344, NVP-LAQ824, Panobinostat, Mocetinostat, and Belinostat.

21. The method of any one of claims 1-20, further comprising (d) assaying a nucleic acid sample obtained from the cancer cells for presence of a B-RAF^{V600E} mutation.

22. The method of any one of claims 1-21, wherein the normal cells are from the subject having cancer.

23. The method of any one of claims 1-21, wherein the normal cells are from a subject that does not have cancer.

24. A method, comprising
administering an effective amount of an HDAC inhibitor alone or together with (a) an effective amount of a RAF inhibitor, (b) an effective amount of a MEK inhibitor, (c) an effective amount of an ERK inhibitor, and/or (d) an effective amount of a RAF inhibitor and a MEK inhibitor to a subject with cancer having an increased gene copy number, mRNA or protein level, or activity of a marker selected from:
(i) GPCRs that activate production of cyclic AMP, and
(ii) GPCR pathway components selected from the group consisting of PKA, FOS, NR4A1, NR4A2, MITF, and a PKA-activated transcription factor that activates FOS, NR4A1, NR4A2, and MITF.

25. A method, comprising
administering to a subject having cancer an effective amount of an HDAC inhibitor together with (a) an effective amount of a RAF inhibitor, (b) an effective amount of a MEK inhibitor, (c) an effective amount of an ERK inhibitor, and/or (d) an effective amount of a RAF inhibitor and a MEK inhibitor.

26. The method of claim 24 or 25, wherein the subject has cancer cells comprising a mutation in B-RAF.

27. The method of claim 26, wherein the subject has cancer cells comprising a B-RAF^{V600E} mutation.

28. The method of any one of claims 24-27, wherein the RAF inhibitor is selected from the group consisting of RAF265, sorafenib, dabrafenib (GSK2118436), SB590885, PLX 4720, PLX4032, GDC-0879 and ZM 336372.

29. The method of any one of claims 24-28, wherein the MEK inhibitor is selected from the group consisting of CI-1040/PD184352, AZD6244, PD318088, PD98059, PD334581, RDEA119, 6-Methoxy-7-(3-morpholin-4-yl-propoxy)-4-(4-phenoxy-phenylamino)-quinoline-3-carbonitrile and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, trametinib (GSK1120212), and ARRY-438162.

30. The method of any one of claims 24-29, wherein the ERK inhibitor is selected from the group consisting of VTX11e, AEZS-131, PD98059, FR180204, and FR148083.

31. The method of any one of claims 24-30, wherein the HDAC inhibitor is selected from the group consisting of Vorinostat, CI-994, Entinostat, BML-210, M344, NVP-LAQ824, Panobinostat, Mocetinostat, and Belinostat.

32. The method of any one of claims 24-31, wherein the subject has innate resistance to the RAF inhibitor or is likely to develop resistance to the RAF inhibitor.

33. The method of any one of claims 24-32, wherein the subject has innate resistance to the MEK inhibitor or is likely to develop resistance to the MEK inhibitor.

34. The method of any one of claims 24-33, wherein the cancer is selected from the group consisting of melanoma, breast cancer, colorectal cancer, glioma, lung cancer, ovarian cancer, sarcoma and thyroid cancer.

35. The method of claim 34, wherein the cancer is melanoma.

36. A method of identifying a marker that confers resistance to a MAPK pathway inhibitor, the method comprising:
culturing cells having sensitivity to a MAPK pathway inhibitor;
expressing a plurality of ORF clones in the cell cultures, each cell culture expressing a different ORF clone;
exposing each cell culture to the MAPK pathway inhibitor; and
identifying cell cultures having greater viability than a control cell culture after exposure to the MAPK pathway inhibitor to identify one or more ORF clones that confers resistance to the MAPK pathway inhibitor.

37. The method of claim 36, wherein the cultured cells have sensitivity to a RAF inhibitor.

38. The method of claim 36, wherein the cultured cells have sensitivity to a MEK inhibitor.

39. The method of claim 36, wherein the cultured cells have sensitivity to an ERK inhibitor.

40. The method of any one of claims 36-39, wherein the cultured cells comprise a B-RAF mutation.

41. The method of claim 40, wherein the cultured cells comprise a B-RAF^{V600E} mutation.

42. The method of any one of claims 36-41, wherein the cultured cells comprise a melanoma cell line.

43. A device comprising:
a sample inlet and a substrate, wherein the substrate comprises a binding partner for a marker selected from:
(i) GEFs selected from the group consisting of ARHGEF2, ARHGEF3, ARHGEF9, ARHGEF19, MCF2L, NGEF, VAV1, PLEKHG3, PLEKHG5, PLEKHG6, IQSEC1, TBC1D3G, SPATA13, RASGRP2, RASGRP3, and RASGRP4,
(ii) GPCRs that activate production of cyclic AMP,
(iii) GPCR pathway components selected from the group consisting of PKA, FOS, NR4A1, NR4A2, MITF, and a PKA-activated transcription factor that activates FOS, NR4A1, NR4A2, and MITF,
(iv) transcription factors selected from the group consisting of POU51, HOXD9, EBF1, HNF4A, SP6, ESRRG, TFEB, FOXA3, FOS, MITF, FOXJ1, XBP1, NR4A1, ETV1, HEY1, KLF6, HEY2, JUNB, SP8, OLIG3, PURG, FOXP2, YAP1, NFE2L1, TLE1, PASD1, TP53, WWTR1, SATB2, NR4A2, HAND2, GCM2, SHOX2, NANOG, CRX, ZNF423, ISX, ETS2, SIM2, MAFB, MYOD1, and HOXC11,
(v) serine/threonine kinases selected from the group consisting of PRKACA, RAF1, NF2, PRKCE, PAK3, and MOS,
(vi) ubiquitin machinery proteins selected from the group consisting of FBX05, TNFAIP1, KLHL10, ARIH1, and TRIM50,
(vii) adaptor proteins selected from the group consisting of CRKL, CRK, TRAF3IP1, FRS3, AND SQSTM1,
(viii) protein tyrosine kinases selected from the group consisting of HCK, BTK, LCK, SRC, and LYNp,
(ix) receptor tyrosine kinases selected from the group consisting of FGR, FGFR2, AXL, and TYRO3,
(x) protein binding proteins selected from the group consisting of CARD9 and WDR5,
(xi) cytoskeletal proteins selected from the group consisting of PVRL1 and TEKT5,
(xii) RNA binding proteins selected from the group consisting of SAMD4B and SAMD4A, and
(xiii) VPS28, IFNA10, KLHL34, TNFRSF13B, CYP2E1, BRMS1L, ADAP2, MLYCD, MAGEA9, RIT2, and KCTD1.

44. A method comprising:
(a) assaying a GEF gene copy number, mRNA or protein level, or activity level of one or more GEFs selected from the group consisting of ARHGEF2, ARHGEF3, ARHGEF9, ARHGEF19, MCF2L, NGEF, VAV1, PLEKHG3, PLEKHG5, PLEKHG6, IQSEC1, TBC1 D3G and SPATA13 in cancer cells from a subject having cancer,
(b) comparing the GEF gene copy number, mRNA or protein level, or activity level in the cancer cells with a GEF gene copy number, mRNA or protein level, or activity level in normal cells, and
(c) identifying a subject having cancer cells with increased GEF gene copy number, mRNA or protein level, or activity level relative to normal cells as a subject (i) who is at risk of developing resistance to a MAPK pathway inhibitor, (ii) who is likely to benefit from treatment with a GEF inhibitor, (iii) who is likely to benefit from treatment with a combination therapy comprising a GEF inhibitor, and/or (iv) who is likely to benefit from treatment with a combination therapy comprising a MAPK pathway inhibitor and a GEF inhibitor.

45. The method of claim 44, wherein the cancer is selected from the group consisting of melanoma, breast cancer, colorectal cancers, glioma, lung cancer, ovarian cancer, sarcoma and thyroid cancer.

46. The method of claim 44, wherein the subject has melanoma.

47. The method of any one of claims 44-46, wherein the cancer cells comprise a mutation in B-RAF.

48. The method of any one of claims 44-47, wherein the cancer cells comprise a V600E B-RAF mutation.

49. The method of any one of claims 44-48, wherein the subject has received a therapy comprising a MAPK pathway inhibitor.

50. The method of claim 49, wherein the subject has manifest resistance to the MAPK pathway inhibitor.

51. The method of claim 44, wherein the subject is likely to develop resistance to a MAPK pathway inhibitor.

52. The method any one of claims 44-51, wherein the MAPK pathway inhibitor is a RAF inhibitor.

53. The method of any one of claims 44-52, wherein the MAPK pathway inhibitor is a pan-RAF inhibitor.

54. The method of any one of claims 44-53, wherein the MAPK pathway inhibitor is a selective RAF inhibitor.

55. The method of claim 54, wherein the RAF inhibitor is selected from the group consisting of RAF265, sorafenib, SB590885, PLX 4720, PLX4032, GDC-0879 and ZM 336372.

56. The method of any one of claims 44-55, wherein the MAPK pathway inhibitor is a MEK inhibitor.

57. The method of any one of claims 44-56, wherein the GEF inhibitor is an inhibitor of ARHGEF2, ARHGEF3, ARHGEF9, ARHGEF19, MCF2L, NGEF, VAV1, PLEKHG3, PLEKHG5, PLEKHG6, IQSEC1, TBC1 D3G and/or SPATA13.

58. The method of any one of claims 44-57, further comprising (d) assaying a nucleic acid sample obtained from the cancer cells for the presence of a mutation in a nucleic acid molecule encoding a B-RAF polypeptide with a mutation at about amino acid position 600.

59. The method of claim 58, further comprising identifying a subject having the mutation in the nucleic acid molecule encoding the B-RAF polypeptide as a subject who is likely to benefit from treatment with the combination therapy.

60. The method of any one of claims 44-59, comprising assaying the gene copy number, the mRNA or the protein level of one or more GEFs.

61. The method of any one of claims 44-60, comprising assaying active status of one or more GTPases.

62. The method of any one of claims 44-61, wherein the normal cells are from the subject having cancer.

63. The method of any one of claims 44-62, wherein the normal cells are from a subject that does not have cancer.

64. A method of treating cancer in a subject, comprising
administering to the subject an effective amount of a GEF inhibitor alone or together with (a) an effective amount of a RAF inhibitor, (b) an effective amount of a MEK inhibitor, or (c) an effective amount of a RAF inhibitor and a MEK inhibitor.

65. A method of treating cancer in a subject comprising
administering, to a subject having an increased GEF gene copy number, mRNA or protein level, or activity, the effective amount of a GEF inhibitor alone or with (i) an effective amount of a RAF inhibitor, (ii) an effective amount of a MEK inhibitor, or (iii) an effective amount of a RAF inhibitor and an effective amount of a MEK inhibitor.

66. The method of claim 64 or 65, wherein the subject has cancer cells comprising a mutation in B-RAF.

67. The method of claim 66, wherein the subject has cancer cells comprising a B-RAF^{V600E} mutation.

68. The method of any one of claims 64-67, wherein the RAF inhibitor is selected from the group consisting of RAF265, sorafenib, SB590885, PLX 4720, PLX4032, GDC-0879 and ZM 336372.

69. The method of any one of claims 64-68, wherein the MEK inhibitor is selected from the group consisting of CI-1040/PD184352, AZD6244, PD318088, PD98059, PD334581, RDEA119, 6-Methoxy-7-(3-morpholin-4-yl-propoxy)-4-(4-phenoxy-phenylamino)-quinoline-3-carbonitrile and 4-[3-Chloro-4-(1-methyl-1H-imidazol-2-ylsulfanyl)-phenylamino]-6-methoxy-7-(3-morpholin-4-yl-propoxy)-quinoline-3-carbonitrile, and ARRY-438162.

70. The method of any one of claims 64-69, wherein the subject has innate resistance to the RAF inhibitor or is likely to develop resistance to the RAF inhibitor.

71. The method of any one of claims 64-70, wherein the subject has innate resistance to the MEK inhibitor or is likely to develop resistance to the MEK inhibitor.

72. The method of any one of claims 64-71, wherein the cancer is selected from the group consisting of melanoma, breast cancer, colorectal cancers, glioma, lung cancer, ovarian cancer, sarcoma and thyroid cancer.

73. The method of any one of claims 64-72, wherein the cancer is melanoma.

74. The method of any one of claims 61-73, wherein the GEF inhibitor is an inhibitor of ARHGEF2, ARHGEF3, ARHGEF9, ARHGEF19, MCF2L, NGEF, VAV1, PLEKHG3, PLEKHG5, PLEKHG6, IQSEC1, TBC1 D3G and/or SPATA13.

75. A method of identifying a GEF target that confers resistance to a MAPK pathway inhibitor, the method comprising:
culturing cells having sensitivity to MAPK pathway inhibitor;
expressing a plurality of GEF ORF clones in the cell cultures, each cell culture expressing a different GEF ORF clone;
exposing each cell culture to the MAPK pathway inhibitor; and
identifying cell cultures having greater viability than a control cell culture after exposure to the MAPK pathway inhibitor to identify one or more GEF ORF clones that confers resistance to the MAPK pathway inhibitor.

76. The method of claim 75, wherein the cultured cells have sensitivity to a RAF inhibitor.

77. The method of claim 75, wherein the cultured cells have sensitivity to a MEK inhibitor.

78. The method of any one of claims 75-77, wherein the cultured cells comprise a B-RAF mutation.

79. The method of any one of claims 75-78, wherein the cultured cells comprise a B-RAF^{V600E} mutation.

80. The method of any one of claims 75-79, wherein the cultured cells comprise a melanoma cell line.
